SEQUENCE LISTING (4 sequences)

SEQ ID NO: 1 - protein, 20 aa, synthetic construct
  REGION 1..20: note = Synthetic
FRNQVDSETHCHAERCFLSW

SEQ ID NO: 2 - protein, 20 aa, synthetic construct
  REGION 1..20: note = Synthetic
FRNQVDSETHCHAQRCFLSW

SEQ ID NO: 3 - DNA, 21 bp, synthetic construct
  misc_feature 1..21: note = Synthetic
  source 1..21: mol_type = other DNA
cctaaggttaagtcgccctcg

SEQ ID NO: 4 - DNA, 41 bp, synthetic construct
  misc_feature 1..41: note = Synthetic
  source 1..41: mol_type = other DNA
ccggacctcctcacgagcccaagttcgtttaccaagcaaaa

(19) United States
(12) Patent Application Publication
(10) Pub. No.: US 2026/0364072 A1
(43) Pub. Date: Nov. 19, 2026

(54) METHODS FOR PROMOTING EXPANSION OF HUMAN CD34+ CELLS IN VIVO AND FOR PROMOTING NEURAL REGENERATION

(71) Applicant: THE REGENTS OF THE UNIVERSITY OF CALIFORNIA, Oakland, CA (US)

(72) Inventors: Catriona JAMIESON, La Jolla, CA (US); Jessica PHAM, La Jolla, CA (US); Christina WU, La Jolla, CA (US); Wenxue MA, La Jolla, CA (US); Jane ISQUITH, La Jolla, CA (US)

(21) Appl. No.: 19/491,473

(22) PCT Filed: Jun. 7, 2024
(86) PCT No.: PCT/US2024/033031

§ 371 Date: Dec. 9, 2025

Related U.S. Application Data
(60) Provisional application No. 63/472,239, filed on Jun. 9, 2023.

(51) Int. Cl. A61K 31/506; A61K 31/365; A61K 31/4155; A61K 31/4184; A61K 31/4439; A61K 31/453; A61K 31/473; A61K 31/517; A61K 31/52; A61K 31/5377; A61K 31/55; A61K 31/706; A61P 1/16; A61P 25/00

(52) U.S. Cl. CPC A61K 31/506; A61K 31/365; A61K 31/4155; A61K 31/4184; A61K 31/4439; A61K 31/453; A61K 31/473; A61K 31/517; A61K 31/52; A61K 31/5377; A61K 31/55; A61K 31/706; A61P 1/16; A61P 25/00

(57) ABSTRACT

In alternative embodiments, provided are methods for promoting expansion of human CD34⁺ cells and sparing CD45⁺ cell survival in vivo, promoting normal hematopoietic stem cell retention in the bone marrow niche, or for treating, preventing or ameliorating a spinal cord injury, liver cirrhosis and peripheral vascular disease, and for promoting neuronal regeneration. In alternative embodiments, provided are methods for treating, preventing or ameliorating a JAK2-related disease or a ADAR-related disease, or a cancer, neoplasm or tumor, comprising administering to an individual in need thereof a drug combination comprising rebecsinib, or rebecsinib and fedratinib.

### RELATED APPLICATIONS

This Patent Convention Treaty (PCT) International Application claims the benefit of priority under 35 U.S.C. § 119(e) of U.S. Provisional Patent Application Ser. No. 63/472,239, filed Jun. 9, 2023. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under NIH/NCAT S UL1TR001442, NIH/NCI R01CA205944, NIH/NIDDK R01DK114468-01, NIH/NCI 2P30CA023100-28, CIRM TRAN1-10540, awarded by the National Institutes of Health (NIH); and NASA NRA-NNJ13ZBG001N. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 6, 2024, is named “0321.152737PCT.xml” and is 5,828 bytes in size. The sequence listing contained in this .XML file is part of the specification and is hereby incorporated by reference herein in its entirety.

### TECHNICAL FIELD

This invention generally relates to biology and cancer treatments. In alternative embodiments, provided are methods for promoting expansion of human CD34⁺ cells in vivo and sparing CD45⁺ cell survival in vivo, promoting normal hematopoietic stem cell retention in the bone marrow niche, or for treating, preventing or ameliorating a spinal cord injury, liver cirrhosis and peripheral vascular disease, and for promoting neuronal regeneration. In alternative embodiments, provided are methods for treating, preventing or ameliorating a JAK2-related disease or a ADAR-related disease, or a cancer, neoplasm or tumor, comprising administering to an individual in need thereof a drug combination comprising rebecsinib and fedratinib.

### BACKGROUND

CD34 is a transmembrane phosphoglycoprotein protein encoded by the CD34 gene in humans, mice, rats and other species. CD34 derives its name from the cluster of differentiation protocol that identifies cell surface antigens. CD34 was first described on hematopoietic stem cells as a cell surface glycoprotein and functions as a cell-cell adhesion factor. It may also mediate the attachment of hematopoietic stem cells to bone marrow extracellular matrix or directly to stromal cells. Clinically, it is associated with the selection and enrichment of hematopoietic stem cells for bone marrow transplants. CD34 expression is express on hematopoietic cells and many other cell types. Injection of CD34+ hematopoietic stem cells has been clinically applied to treat various diseases including neuronal regeneration and spinal cord injury, liver cirrhosis and peripheral vascular disease. CD34 has been shown to interact with CRKL and L-selectin, is important in inflammation and neuronal regeneration.

### SUMMARY

In alternative embodiments, provided are methods for promoting expansion of human CD34⁺ cells in vivo and sparing CD45⁺ cell survival in vivo, promoting normal hematopoietic stem cell retention in the bone marrow niche, or for treating, preventing or ameliorating a spinal cord injury, liver cirrhosis and peripheral vascular disease, and for promoting neuronal regeneration. In alternative embodiments, provided are methods for treating, preventing or ameliorating a JAK2-related disease or a ADAR-related disease, or a cancer, neoplasm or tumor, comprising administering to an individual in need thereof a drug combination comprising rebecsinib, or rebecsinib and fedratinib (optionally INREBIC™).

In alternative embodiments, provided are methods and combination therapies, and therapeutic combinations, comprising rebecsinib, or fedratinib (optionally INREBIC™) and rebecsinib, and this new drug combination can modulate the spliceosome and has the ability to down-regulate levels of ADAR enzymes. In alternative embodiments, this new drug combination is useful in promoting expansion of human CD34⁺ cells for treating, preventing or ameliorating a spinal cord injury, liver cirrhosis and peripheral vascular disease, and for promoting neuronal regeneration. In alternative embodiments, this new drug combination is useful in addressing JAK2-related diseases, ADAR-related diseases, or both, including diseases and conditions such as neoplasms, tumors and cancers, including cancers lacking receptors for estrogen, progesterone, and HER2 (human epidermal growth factor receptor 2; or CD340 (cluster of differentiation 340)), such as triple negative breast cancer.

In alternative embodiments, provided are methods for:
promoting in vivo expansion of human CD34⁺ cells and sparing CD45⁺ cell survival in vivo in an individual in need thereof,
treating, preventing or ameliorating a spinal cord injury, liver cirrhosis and/or peripheral vascular disease, and/or for promoting neuronal (neural) regeneration in an individual in need thereof,
treating, preventing or ameliorating a stroke, or treating, preventing or ameliorating a thrombo-occlusive cerebrovascular disease in an individual in need thereof,
increasing human CD34⁺ cells and normal hematopoietic stem cells (HSCs) (or increasing human CD34⁺ cells or HSC cell numbers) in an in vivo bone marrow microenvironment in an individual in need thereof, optionally increasing by at least about 10% to 100%,
promoting normal hematopoietic stem cell retention in the bone marrow niche in vivo in an individual in need thereof,
treating a JAK2-related disease or a ADAR-related disease in an individual in need thereof, or
treating, preventing or ameliorating a cancer, neoplasm or tumor in an individual in need thereof,
the method comprising administering to an individual in need thereof a drug combination comprising: rebecsinib, or rebecsinib and fedratinib (optionally INREBIC™),
wherein optionally the rebecsinib, or rebecsinib and fedratinib (optionally INREBIC™), or any combination of drugs as provided herein, is administered to an individual in need thereof after a stroke or any thrombo-occlusive cerebrovascular evert, or to treat or ameliorate a thrombo-occlusive cerebrovascular disease;
wherein optionally the rebecsinib, or rebecsinib and fedratinib (optionally INREBIC™), or any combination of drugs as provided herein, is administered to an individual in need thereof after a neural trauma event, optionally a spinal chord injury, or after a trauma or surgery that damages or severs a nerve or the spinal chord,
wherein optionally the rebecsinib, or rebecsinib and fedratinib (optionally INREBIC™), or any combination of drugs as provided herein, is administered to an individual in need thereof having any acute or chronic neurodegeneration or disease associated with a neurodegenerative pathology, such as for example Parkinson's disease or Alzheimer's disease, or traumatic brain injury (TBI) or Chronic Traumatic Encephalopathy (CTE), or chemical brain or nerve damage,
wherein optionally the individual in need thereof is an individual that has or has had an acute or chronic neurodegeneration or disease associated with a neurodegenerative pathology, optionally Parkinson's disease or Alzheimer's disease, or traumatic brain injury (TBI) or Chronic Traumatic Encephalopathy (CTE), or chemical brain or nerve damage,
wherein optionally the individual in need thereof is an individual that has or has had a liver disease or infection (viral or parasitic infection, or fasciolosis), or liver cancer or liver infection (optionally hepatitis), or a fatty liver or has fatty liver disease (hepatic steatosis), autoimmune hepatitis, alcoholic hepatitis, or Nonalcoholic Fatty Liver Disease.

In alternative embodiments of methods as provided herein:
the in vivo expansion of human CD34⁺ cells and sparing CD45⁺ cell survival in vivo is used for treating, preventing or ameliorating a spinal cord injury, liver cirrhosis and/or a peripheral vascular disease, and/or for promoting neuronal regeneration;
the cancer, neoplasm or tumor is a cancer lacking receptors for estrogen, progesterone and/or HER2 (human epidermal growth factor receptor 2, or lacking CD340 (cluster of differentiation 340)), or is a triple negative breast cancer;
doses of rebecsinib, or rebecsinib and fedratinib, are administered, or formulated for administration, once a day for between one to two weeks, twice a week for 2 weeks or between about one to two weeks, followed by 2 weeks rest or 2 to 4 weeks rest, with a duration of two, three, four, five or six cycles, optionally with a duration of four 28 day or monthly cycles;
the method further comprises administering to an individual in need thereof an ATP-competitive protein tyrosine kinase inhibitor, wherein optionally the ATP-competitive protein tyrosine kinase inhibitor comprises dasatinib (or SPRYCEL™ or DASANIX™);
doses of rebecsinib, or rebecsinib and fedratinib, are dosaged at about 60 mg/kg twice daily orally, or between about 20 mg to 100 mg, optionally for one to two, or three, or four, or five or more weeks, or is dosaged at between about 10 to 500 mg/day, or between about 500 to 1 gram a day, or at a dosage of between about 100 to 600 mg per day or per dosage, or at about 100, 200, 300, 400, 500 or 600 mg per day or per dosage, and optionally a unit dosage is administered to an individual in need thereof once a day (QD), or twice a day (BID), or three times a day (TID), or more;
the method further comprises administering to the individual in need thereof one, two, three or more of: a hypomethylating agent (HMA), wherein optionally the HMA comprises azacitidine (or VIDAZA™) or decitabine (or DACOGEN™) afatinib (or GILOTRIF™), afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, fedratinib (or INREBIC™), floxuridine, fludarabine, fluorouracil, gefitinib, gemcitabine, hydroxyurea, ibrutinib, icotinib, idarubicin, ifosfamide, imatinib, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib (or OPZELURA™), seliciclib, sorafenib (or NEXAVAR™), streptozocin, sulfatinib, sunitinib (or SUTENT™) tamoxifen (or NOLVADEX™), tandutinib, temozolomide, temsirolimus, teniposide, theliatinib, thioguanine, thiotepa, topotecan, uramustine, valrubicin, vandetanib, vemurafenib (or ZELBORAE™), vincristine (or ONCOVIN™), vinblastine (or VELBAN™), vinorelbine (or NAVELBINE™), and vindesine (or eldisine);
the method further comprises administering to the individual in need thereof comprises a telomerase inhibitor, wherein optionally the telomerase inhibitor comprises at least one, two or three of: imetelstat, zidovudine (or azidothymidine (AZT)), stavudine (or ZERIT™), tenofovir or tenofovir disoproxil (or VIREAD™), didanosine (or VIDEX™), abacavir (ZIAGEN™), TMPI, telomestatin, RHPS4, BRACO-19, TMPyP4, tertomotide, ASTVAC-1, GX-301, UCPVax, UV-1, Vx-001, Vx-006, INO-1400, INVAC-1, ASTVAC-2, Telin(ab 4,4-dichloro-1-(2,4-dichlorophenyl)-3-methyl-5-pyrazolone), Vbx-011, Vbx-021, Vbx-026INO-5401, KML-001, TK-005, ribovax, Vbx-016, ZI-HX, ZI-H04, and ZIH-03;
the formulation, pharmaceutical composition or therapeutic combination of drugs or an active agent or drug contained therein administered to the individual in need thereof is or are formulated or contained in: a liquid formulation (optionally sterile saline or water), a spray, a powder, an aerosol, a mist, or any formulation for inhalation, a pill, a capsule, a tablet, or a geltab, or equivalents; or, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; or, a drug delivery device or package, blister pack, clamshell or tray comprising a plurality of compartments spatially arranged on the drug delivery device or package, blister pack, clamshell or tray to follow a dosage administration regimen;
an active agent or drug in the formulation, pharmaceutical composition or therapeutic combination of drugs administered to the individual in need thereof is dosaged at between about 10 to 500 mg/day, or between about 500 to 1 gram a day, or at a dosage of between about 100 to 600 mg per day or per dosage, or at about 100, 200, 300, 400, 500 or 600 mg per day or per dosage, and optionally a unit dosage is administered to an individual in need thereof once a day (QD), or twice a day (BID), or three times a day (TID), or more;
an active agent or drug in the formulation, pharmaceutical composition or therapeutic combination of drugs administered to the individual in need thereof is administered as or formulated with or formulated as an) inhaled or aerosol formulation such as a powder or a mist or aerosol, and/or is formulated with or formulated as an oral, intramuscular (IM), subcutaneous (SC), intrathecal or intravenous (IV) formulation, wherein optionally both the inhaled (or aerosol) and the oral, IV, SC, intrathecal and/or IM formulations are administered simultaneously or sequentially; and/or
an active agent or drug in the formulation, pharmaceutical composition or therapeutic combination of drugs administered to the individual in need thereof using a drug delivery device, optionally by inhalation, wherein the drug delivery device optionally comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device, or intravenously (IV) or intramuscularly (IM).

In alternative embodiments, drug formulations or drug combinations as provided herein are administered to an individual in need thereof after a stroke, or to treat or ameliorate a thrombo-occlusive cerebrovascular disease.

In alternative embodiments, provided are drug combination comprising: rebecsinib, or rebecsinib and fedratinib, for use in.
promoting in vivo expansion of human CD34⁺ cells and sparing CD45⁺ cell survival in vivo in an individual in need thereof,
increasing human CD34⁺ cells and normal hematopoietic stem cells (HSCs) in an in vivo bone marrow microenvironment in an individual in need thereof,
treating, preventing or ameliorating a spinal cord injury, liver cirrhosis and/or peripheral vascular disease, and/or for promoting neuronal regeneration, in an individual in need thereof,
treating, preventing or ameliorating a stroke, or treating, preventing or ameliorating a thrombo-occlusive cerebrovascular disease in an individual in need thereof,
promoting normal hematopoietic stem cell retention in the bone marrow niche in vivo in an individual in need thereof,
treating a JAK2-related disease or a ADAR-related disease in an individual in need thereof, or
treating, preventing or ameliorating a cancer, neoplasm or tumor in an individual in need thereof,
wherein optionally the individual in need thereof is an individual that has or has had an acute or chronic neurodegeneration or disease associated with a neurodegenerative pathology, optionally Parkinson's disease or Alzheimer's disease, or traumatic brain injury (TBI) or Chronic Traumatic Encephalopathy (CTE), or chemical brain or nerve damage,
wherein optionally the individual in need thereof is an individual that has or has had a liver disease or infection (viral or parasitic infection, or fasciolosis), or liver cancer or liver infection (optionally hepatitis), or a fatty liver or has fatty liver disease (hepatic steatosis), autoimmune hepatitis, alcoholic hepatitis, or Nonalcoholic Fatty Liver Disease.

In alternative embodiments, provided are uses of a drug combination comprising: rebecsinib, or rebecsinib and fedratinib, for:
promoting in vivo expansion of human CD34⁺ cells and sparing CD45⁺ cell survival in vivo in an individual in need thereof,
increasing human CD34⁺ cells and normal hematopoietic stem cells (HSCs) in an in vivo bone marrow microenvironment in an individual in need thereof,
treating, preventing or ameliorating a spinal cord injury, liver cirrhosis and/or peripheral vascular disease, and/or for promoting neuronal regeneration, in an individual in need thereof,
treating, preventing or ameliorating a stroke, or treating, preventing or ameliorating a thrombo-occlusive cerebrovascular disease in an individual in need thereof,
promoting normal hematopoietic stem cell retention in the bone marrow niche in vivo in an individual in need thereof,
treating a JAK2-related disease or a ADAR-related disease in an individual in need thereof, or
treating, preventing or ameliorating a cancer, neoplasm or tumor in an individual in need thereof,
wherein optionally the individual in need thereof is an individual that has or has had an acute or chronic neurodegeneration or disease associated with a neurodegenerative pathology, optionally Parkinson's disease or Alzheimer's disease, or traumatic brain injury (TBI) or Chronic Traumatic Encephalopathy (CTE), or chemical brain or nerve damage,
wherein optionally the individual in need thereof is an individual that has or has had a liver disease or infection (viral or parasitic infection, or fasciolosis), or liver cancer or liver infection (optionally hepatitis), or a fatty liver or has fatty liver disease (hepatic steatosis), autoimmune hepatitis, alcoholic hepatitis, or Nonalcoholic Fatty Liver Disease.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A-D illustrate data demonstrating that rebecsinib treatment promotes human CD34⁺ cells and spares CD45⁺ cells:

FIG. 1A illustrates integrated fluorescence system/laser (IVIS 200™ (Xenogen)) images of NSG-SGM3 mice intravenously transplanted with ADAR1-NanoLuc reporter transduced normal human CD34⁺ cells at 18 weeks after transplantation;

FIG. 1B graphically illustrates flow cytometry gating strategies showing positive and negative controls;

FIG. 1C graphically illustrates data showing treatment with Rebecsinib, a selective small molecule splicing modulator, leads to increased levels of human CD34⁺ cells in PB, SP, and a significant CD34⁺ increase in BM cells; and

FIG. 1D graphically illustrates data showing rebecsinib treatment preserves human CD45⁺ cells and differentiated cells, including CD14⁺ myeloid cells, and lymphoid cells such as CD3⁺ T and CD19⁺ B cells;

as discussed in further detail in Example 1, below.

FIG. 2A-B graphically illustrate data showing rebecsinib treatment inhibits CD34⁺Lin⁻ cells in sAML PDX models:

FIG. 2A graphically illustrates data demonstrating that the CD34⁺Lin⁻ cell population exhibited significant inhibition in both PB and SP (p=0.0015 and p=0.0006, respectively; and

FIG. 2B graphically illustrates data demonstrating that rebecsinib treatment preserved human CD45+ cells in the organs of PB, BM, and SP,

as discussed in further detail in Example 1, below.

FIG. 3 graphically illustrates data demonstrating that rebecsinib treatment significantly increases sAML PDX survival, where sAML mouse models were randomly grouped into 4 treatment conditions including (1) vehicle treatment, (2) single drug treatment with fedratinib, (3) single drug treatment with rebecsinib, and (4) combination treatment with both drugs, as discussed in further detail in Example 1, below.

FIG. 4A-F graphically illustrate data from studies where MDA-MB-231 cells were intrahepatically transplanted into Rag2 neonatal mice, then treated with fedratinib, rebecsinib, or a combination of fedratinib and rebecsinib, and data demonstrates that the combination of fedratinib and rebecsinib significantly inhibited CD44+ breast cancer cells in lung (FIG. 4A), spleen (FIG. 4B), bone marrow (FIG. 4C), liver (FIG. 4D), spinal cord (FIG. 4E) and peripheral blood (FIG. 4F), as discussed in further detail in Example 4, below.

FIG. 5 graphically illustrates IVIS (In Vivo Imaging System) results of ADAR1-MDA-MB-231 transplanted Rag2 mouse models, and IVIS images were taken at 6 weeks after transplant (FIG. 5A), after two weeks of therapy (FIG. 5B), and one week after therapy was completed (FIG. 5C), as discussed in further detail in Example 5, below.

FIG. 6A-E illustrate different innate immune deaminases: APOBEC3 and ADAR1:

FIG. 6A schematically illustrates the deamination enzymatic activity of APOBEC3;

FIG. 6B schematically illustrates the seven major innate homologs of APOBEC3, and catalytic activity is restricted to the C-terminal, while RNA binding activity in all domains remains functional;

FIG. 6C schematically illustrates the enzymatic activity of ADAR1 converting adenosine to inosine;

FIG. 6D schematically illustrates different innate isoforms of ADAR1, ADAR1 has 2 isoforms: the constitutively expressed p110 isoform, and the interferon inducible p150 isoform; and

FIG. 6E illustrate an immunofluorescence image: Colocalization of APOBEC3C and ADAR1 in TF1a cells: immunofluorescence of anti-APOBEC3C (green) and anti-ADAR1 p150-specific (red) antibodies in TF1a shADAR1 and TF1a shControl knockdown cells demonstrate a colocalization (yellow) of APOBEC3C and ADAR1 p150 proteins in the shControl cells; TF1a shADAR1 cells show ablation of ADAR1 protein,

as discussed in further detail in Example 2, below.

FIG. 7 A-F illustrate differential expression of APOBEC3 and ADAR1 in hematopoietic malignancies, and differential expression of APOBEC3s in multiple disease states as compared to young and aged normal peripheral blood:

FIG. 7A schematically illustrates normal hematopoiesis;

FIG. 7B is a table illustrating a clinical characterization of myeloproliferative neoplasms;

FIG. 7C graphically illustrates APOBEC3 expression in progenitor cell populations in normal young and aged samples and across the myeloproliferative neoplasm disease spectrum, where APOBEC3C (green) shows the highest expression of all the APOBEC3 genes in both progenitor and stem cell populations;

FIG. 7D graphically illustrates APOBEC3C expression in the stem cell populations of normal young and aged samples and myeloproliferative neoplasms;

FIG. 7E graphically illustrates ADAR1 expression in the stem population of aged and young bone marrow, and in myeloproliferative neoplasm patient samples; and

FIG. 7F graphically illustrates ADAR1 expression in the stem population of young and aged normal controls, and in myeloproliferative neoplasm patient samples,

as discussed in further detail in Example 2, below.

FIG. 8A-C illustrates APOBEC3 lentiviral overexpression, and construction and modification of APOBEC3C plasmids:

FIG. 8A schematically illustrates a vector map of APOBEC3C lentiviral vector, where FLAG-tags were added to constructs for use in pull-down experiments;

FIG. 8B schematically illustrates a crystal Structure of APOBEC3C, with the active site, Glu68 highlighted;

FIG. 8C illustrates how a APOBEC3C E68Q vector was created using site-directed mutagenesis and introducing a g202c point mutation, where this mutation functionally replaces Glutamic acid (Glu) 68 by Glutamine (Gln) and renders APOBEC3C catalytically inactive:

SEQ ID NO: 1 is
FRNQVDSETHCHAERCFLSW;
SEQ ID NO: 2 is
FRNQVDSETHCHAQRCFLSW,

as discussed in further detail in Example 2, below.

FIG. 9A-H illustrate APOBEC3 overexpression and differential gene expression changes:

FIG. 9A graphically illustrates relative APOBEC3C overexpression in CD34+ cord blood;

FIG. 9B graphically illustrates an MA plot of APOBEC3C overexpression;

FIG. 9C illustrates a heatmap showing top 50 differentially expressed genes after APOBEC3C overexpression;

FIG. 9D illustrates a Venn diagram of APOBEC3 differentially expressed genes;

FIG. 9E illustrates a table showing unique differentially expressed genes for each APOBEC3 family member;

FIG. 9F illustrates a table showing unique differential expression and fold change of genes after APOBEC3C overexpression, where red represents significantly upregulated genes; blue represents significantly downregulated genes;

FIG. 9G graphically illustrates ADAR1 p150/p110 expression after APOBEC3 overexpression compared to pCDH backbone control; and

FIG. 9H graphically illustrates significantly enriched pathways found with GSEA REACTOME™ analysis after APOBEC3C overexpression, where five major pathways were significantly enriched after A3C overexpression,

as discussed in further detail in Example 2, below.

FIG. 10A-I illustrate editing profiles of normal and malignant hematopoietic cells:

FIG. 10A-C graphically illustrate C-to-U RNA editing in APOBEC3C overexpressed normal CD34+ cord blood cells: variant allele frequency (FIG. 10A), edited genes per sample (FIG. 10B), editing stratified by variant classification (FIG. 10C);

FIG. 10D-F graphically illustrate A-to-I RNA editing in APOBEC3C overexpressed normal CD34+ cord blood cells: variant allele frequency (FIG. 10D); A-to-I edits per million reads (FIG. 10E), and editing stratified by variant classification (FIG. 10F);

FIG. 10G graphically illustrates overall A-to-I RNA editing frequency by MPN stage;

FIG. 10H graphically illustrates a boxplot depicting the number of somatic DNA mutations in peripheral blood or saliva based on transitions (Tis) or transversions (Tvs) from myeloproliferative neoplasm patient samples, both somatic and germline variants were included; and

FIG. 10I graphically illustrates a boxplot depicting the number of somatic DNA mutations in peripheral blood or saliva based on transitions (Tis) or transversions (Tvs), both somatic and germline variants were included,

as discussed in further detail in Example 2, below.

FIG. 11A-C illustrates prognostic implications for enhanced leukemic transformation and poor prognosis after deregulation of APOBEC3C and ADAR1, both innate immune deaminases:

FIG. 11A illustrates a Kaplan-Meier plot showing overall survival of AML patients with high expression of APOBEC3C (red) or low expression of APOBEC3C (blue);

FIG. 11B illustrates a Kaplan-Meier plot showing overall survival of AML patients with high or low ADAR1 expression (red) or low ADAR1 expression (blue);

FIG. 11C illustrates a data showing correlation of APOBEC3C with ADAR1 p150 isoform in stem cells, and points are colored by phenotype,

as discussed in further detail in Example 2, below.

FIG. 12 A-C illustrate that high levels of ADAR1 correlate with shorter OS in high-grade BC:

FIG. 12A illustrates how ADAR1 deaminase activity converts adenosines to inosines which are interpreted as guanosines, de facto changing the mRNA;

FIG. 12B schematically illustrates the ADAR1 two main isoforms p110 and interferon-inducible p150 which expresses the Z-α binding domain for interacting with RNA/DNA in a conformation;

FIG. 12C graphically illustrates data showing reduced Overall Survival (OS) from mRNA dataset of grade three, HER2 positive BC tumors which underwent endocrine therapies (p=0.019), (left image) and reduced OS from protein dataset in grade three BC compared with control (p=0031), (right image), as discussed in further detail in Example 3, below.

FIG. 13A-B illustrate workflow to establish ADAR1-driven brain metastases mouse model:

FIG. 13A schematically illustrates cancer cells transduced with a nanoluc-luciferase reporter construct for tracing ADAR1 activity, and thereafter neonatal pups are implanted through ICV with transduced cells, and tumor growth is for 3-6 weeks for the MDA-MD-231 cells; and

FIG. 13B illustrates a FACS-sorting of GFP+-tumor cells, with cells collected for downstream analyses,

as discussed in further detail in Example 3, below.

FIG. 14A-E illustrate that ADAR1 potentiates cancer stem cells aggressiveness:

FIG. 14A illustrates pictures of MDA-MD-231 ADAR1-reporter metastases from organs (top and middle panel) and digested tissue (bottom panel);

FIG. 14B illustrates a picture of a MDA-MD-231 implanted brain at three weeks post-ICV;

FIG. 14C illustrates a FACS analysis of one brain and one spine from total brain extract co-expressing ADAR1-GFP positive cells, CD44 and CD47;

FIG. 14D illustrates a FACS contour plot of CD44 and CD47 displaying different populations among the parental cell line versus brain and spine metastases; and

FIG. 14E graphically illustrates RT-PCR from GFP-FACS-sorted brain metastases (231-Br S1,S2 and S3) and non-transplanted cell line (231-Cells),

as discussed in further detail in Example 3, below.

FIG. 15A-E illustrate ADAR1 knock-down reduces CD47 expression:

FIG. 15A illustrates pictures of IF of ADAR1 in the MDA-MD-231 cells transduced with sh-scramble (Ctrl) or sh-ADAR1 (ADAR1-KD)

FIG. 15B graphically illustrates RT-PCR confirming down-regulation of ADAR1 p110 and p 150 isoforms, (Two-tailed t-test p value=0.0348, for p110 and 0.0467 for p150);

FIG. 15C illustrates WB showing decreased protein levels in two MDA-MD-231 clones compared with the parental and transduced line;

FIG. 15D graphically illustrates RT-PCR from three independent replicates of multiple genes involved in the regulation of BC metastases; and

FIG. 15E graphically illustrates CD47 KD confirmed via RT-PCR in Ctrl and ADAR1-KD (left), CD47-FACS histogram (right),

as discussed in further detail in Example 3, below.

FIG. 16A-B illustrate characterizing samples from breast cancer metastatic sites:

FIG. 16A illustrates a table showing metastatic apheresis (MBC003) and malignant pleural effusions (mPE) (others) patient specification table; and

FIG. 16B illustrates an example of breast cancer stem cells profiling for the MBC006,

as discussed in further detail in Example 3, below.

FIG. 17A-C illustrate tumoroids grown in a bioreactor bag, an augmented in vitro model:

FIG. 17A illustrates images of exemplary bioreactor bags for BC primary tumoroids from the mPE MB6006 with pCDH-GFP (top) and Br-231 cells (bottom);

FIG. 17B illustrates pictures of MBC006 cells grown in BC conditional media, objective 20×; and

FIG. 17C illustrates pictures of FACS-sorted Br-231 cells with the ADAR1 reporter grown in neural conditional media,

as discussed in further detail in Example 3, below.

FIG. 18A-F graphically illustrate data demonstrating that rebecsinib treatment spares human CD45+ADAR1−GFP+ cells and CD45+CD3+ADAR1−GFP+ cells in aNBM SAK148 PDX mouse peripheral blood (PB), spleen (SP), and bone marrow (BM):

FIG. 18A graphically illustrates levels of CD45+GFP+ cells in PB after rebecsinib treatment;

FIG. 18B graphically illustrates levels of CD45+GFP+ cells in SP after rebecsinib treatment;

FIG. 18C graphically illustrates levels of CD45+GFP+ cells in BM after rebecsinib treatment;

FIG. 18D graphically illustrates levels of CD45+CD3+GFP+ cells in PB after rebecsinib treatment;

FIG. 18E graphically illustrates levels of CD45+CD3+GFP+ cells in SP after rebecsinib treatment; and

FIG. 18F graphically illustrates levels of CD45+CD3+GFP+ cells in BM after rebecsinib treatment,

as discussed in further detail in Example 6, below.

FIG. 19A-F graphically illustrate data demonstrating that rebecsinib treatment (tx) Spares human CD45+ADAR1−GFP+ Cells and CD45+CD3+ADAR1−GFP+ Cells in aNBM SAK148 PDX Mouse PB:

FIG. 19A-B illustrate cell sorting (FACS) scans illustrating levels of CD45+GFP+ cells after rebecsinib treatment;

FIG. 19C-D illustrate cell sorting (FACS) scans illustrating levels of CD45+CD3+GFP+ cells after rebecsinib treatment;

FIG. 19E graphically illustrates data from the cell sorting (FACS) scans illustrated in FIG. 19A-B; and

FIG. 19F graphically illustrates data from the cell sorting (FACS) scans illustrated in FIG. 19C-D,

as discussed in further detail in Example 7, below.

FIG. 20A-F graphically illustrate data demonstrating that rebecsinib treatment (tx) spares human CD45+ADAR1−GFP+ cells and CD45+CD3+ADAR1−GFP+ cells in aNBM SAK148 PDX mouse SP:

FIG. 20A-B illustrate cell sorting (FACS) scans illustrating levels of CD45+GFP+ cells after rebecsinib treatment;

FIG. 20C-D illustrate cell sorting (FACS) scans illustrating levels of CD45+CD3+GFP+ cells after rebecsinib treatment;

FIG. 20E graphically illustrates data from the cell sorting (FACS) scans illustrated in FIG. 20A-B; and

FIG. 20F graphically illustrates data from the cell sorting (FACS) scans illustrated in FIG. 20C-D,

as discussed in further detail in Example 8, below.

FIG. 21A-F graphically illustrate data demonstrating that rebecsinib treatment (tx) spares human CD45+ADAR1−GFP+ cells and CD45+CD3+ADAR1−GFP+ cells in aNBM SAK148 PDX mouse BM:

FIG. 21A-B illustrate cell sorting (FACS) scans illustrating levels of CD45+GFP+ cells after rebecsinib treatment;

FIG. 21C-D illustrate cell sorting (FACS) scans illustrating levels of CD45+CD3+GFP+ cells after rebecsinib treatment;

FIG. 21E graphically illustrates data from the cell sorting (FACS) scans illustrated in FIG. 21A-B; and

FIG. 21F graphically illustrates data from the cell sorting (FACS) scans illustrated in FIG. 21C-D,

as discussed in further detail in Example 9, below.

FIG. 22A-F illustrate data demonstrating that rebecsinib treatment (tx) spares human CD45+ADAR1−GFP+ cells and CD45+CD3+ADAR1−GFP+ cells in aNBM SAK378 PDX Mouse PB, SP, and BM.

FIG. 22A illustrates CD45+GFP+ cells in PB after either vehicle or rebecsinib treatment;

FIG. 22B illustrates CD45+GFP+ cells in SP after either vehicle or rebecsinib treatment;

FIG. 22C illustrates CD45+GFP+ cells in BM after either vehicle or rebecsinib treatment;

FIG. 22D illustrates CD45+CD3+GFP+ cells in PB after either vehicle or rebecsinib treatment;

FIG. 22E illustrates CD45+CD3+GFP+ cells in SP cells in PB after either vehicle or rebecsinib treatment; and

FIG. 22F illustrates CD45+CD3+GFP+ cells in BM cells in PB after either vehicle or rebecsinib treatment,

as discussed in further detail in Example 10, below.

FIG. 23A-F graphically illustrate data demonstrating that rebecsinib treatment (tx) spares human CD45+ADAR1−GFP+ cells and CD45+CD3+ADAR1−GFP+ cells in aNBM SAK378 PDX Mouse PB:

FIG. 23A-B illustrate cell sorting (FACS) scans illustrating levels of CD45+GFP+ cells after rebecsinib treatment;

FIG. 23C-D illustrate cell sorting (FACS) scans illustrating levels of CD45+CD3+GFP+ cells after rebecsinib treatment;

FIG. 23E graphically illustrates data from the cell sorting (FACS) scans illustrated in FIG. 23A-B; and

FIG. 23F graphically illustrates data from the cell sorting (FACS) scans illustrated in FIG. 23C-D,

as discussed in further detail in Example 11, below.

FIG. 24A-F graphically illustrate data demonstrating that rebecsinib treatment (tx) spares human CD45+ADAR1−GFP+ cells and CD45+CD3+ADAR1−GFP+ cells in aNBM SAK378 PDX Mouse SP:

FIG. 24A-B illustrate cell sorting (FACS) scans illustrating levels of CD45+GFP+ cells after rebecsinib treatment;

FIG. 24C-D illustrate cell sorting (FACS) scans illustrating levels of CD45+CD3+GFP+ cells after rebecsinib treatment;

FIG. 24E graphically illustrates data from the cell sorting (FACS) scans illustrated in FIG. 24A-B; and

FIG. 24F graphically illustrates data from the cell sorting (FACS) scans illustrated in FIG. 24C-D,

as discussed in further detail in Example 12, below.

FIG. 25A-F graphically illustrate data demonstrating that rebecsinib treatment (tx) spares human CD45+ADAR1−GFP+ cells and CD45+CD3+ADAR1−GFP+ cells in aNBM SAK378 PDX Mouse BM:

FIG. 25A-B illustrate cell sorting (FACS) scans illustrating levels of CD45+GFP+ cells after rebecsinib treatment;

FIG. 25C-D illustrate cell sorting (FACS) scans illustrating levels of CD45+CD3+GFP+ cells after rebecsinib treatment;

FIG. 25E graphically illustrates data from the cell sorting (FACS) scans illustrated in FIG. 25A-B; and

FIG. 25F graphically illustrates data from the cell sorting (FACS) scans illustrated in FIG. 25C-D,

as discussed in further detail in Example 13, below.

FIG. 26A-C illustrate quantification of ADAR1p150 by Splice Isoform RNA Sequencing (RNA-seq):

FIG. 26A illustrates a RNA-seq-based quantification (counts per million, CPM) of ADAR-201, ADAR-202, and ADAR-208 was performed on FACS-purified hematopoietic stem cells (HSC, CD34⁺CD38⁻Lin⁻) from young (YBM) and aged bone marrow (ABM) HSC, polycythemia vera (PV), essential thrombocythemia (ET), myelofibrosis (MF), chronic myeloid leukemia (CML), or secondary acute myeloid leukemia (sAML). RNA-seq analyses were also performed on FACS-purified hematopoietic progenitor cells (HPC, CD34⁺CD38⁺Lin⁻) from primary samples, including YBM, ABM, PV, ET, MF, CML, de novo (dnAML and sAML) AML;

FIG. 26B illustrates a structural diagram showing the spliceosome core complex with Rebecsinib interacting at the interface of SF3B1 and PHF5A, adapted from the spliceosome complex bound to pladienolide B; and

FIG. 26C illustrates a schematic diagram of the primary ADAR1 p150-encoding transcript, ADAR-202, and proposed Rebecsinib-induced intron retention reducing transcript expression after treatment,

as discussed in further detail in Example 14, below.

FIG. 27A-G illustrates development of a lentiviral ADAR1 A-to-I RNA editing reporter:

FIG. 27A illustrates a schematic diagram demonstrating the synthetic RNA sequence containing an ADAR1-sensitive stop codon that, upon A-to-I editing, reads through to produce nanoluciferase and GFP proteins separated by a T2A cleavage site;

FIG. 27B illustrates ADAR protein expression levels in 293T cells co-transfected with the ADAR1 nanoluciferase-GFP (nanoluc-GFP) reporter and increasing amounts of FLAG-tagged wild-type (WT) ADAR1, catalytically inactive mutant ADAR1 (E912A), or wild-type ADAR2. β-actin was used as a loading control;

FIG. 27C illustrates (C) relative luciferase signals in 293T cells prepared as in FIG. 26B;

FIG. 27D illustrates live cell fluorescent imaging of GFP expression in human myeloid leukemia TF-1a cells transduced with the ADAR1 nanoluc-GFP reporter vector (lower panels) compared to untransduced controls (upper panels);

FIG. 27E illustrates detection of nanoluciferase expression via in vivo bioluminescence (IVIS) imaging of no transplant control (far left), K562-nanoluc-GFP and pCDH vector transduced and K562-nanoluc-GFP and ADAR1 wild-type or E912A mutant transduced human leukemia cells (K562) transplanted into RAG2^{-/-}γc^{-/-} mice; and

FIG. 27F illustrates luminescence-based quantification of ADAR1-dependent nanoluciferase signals in CD34⁺ cells from primary, high-risk MF samples (*=untreated patient) after in vitro transduction with the ADAR1 nanoluc-GFP reporter and treatment with vehicle control (DMSO) or Rebecsinib (72 hr);

as discussed in further detail in Example 14, below.

FIG. 28A-E illustrates data showing that rebecsinib inhibits ADAR1p150 mediated high-risk MF HPC and LSC survival:

FIG. 28A illustrates a schematic diagram of in vitro MF HPC and LSC survival and self-renewal assays;

FIG. 28B illustrates a flow cytometry-based viable cell counts (5,000 events measured) in high-risk MF samples after in vitro treatment of primary CD34+ cells with vehicle control (DMSO) or Rebecsinib (72 hr);

FIG. 28C illustrates a flow cytometry-based quantification of ADAR1 p150 protein expression in high-risk MF samples after in vitro transduction of primary CD34⁺ cells with ADAR1 nanoluc-GFP reporter or vector control (pCDH) followed by treatment with vehicle control (DMSO) or Rebecsinib (72 hr); and

FIG. 28D-E illustrates quantification of colony formation (survival, FIG. 28D) and replating (self-renewal, FIG. 28E) of high-risk MF HPC and sAML LSC compared with cord blood (CB) and aged versus young normal bone marrow (a-NBM, y-NBM) controls treated with Rebecsinib at increasing concentrations;

as discussed in further detail in Example 14, below.

FIG. 29A-H illustrate rebecsinib pharmacodynamic and pharmacokinetic studies in pre-clinical and pre-IND models:

FIG. 29A illustrates quantification of cell viability (left panel) and splicing modulation (RFP/GFP ratios, right panel) by flow cytometry analyses of the human AML cell line (KG-1a) stably transduced with a lentiviral dual-fluorescence splicing reporter vector and treated with increasing concentrations of Rebecsinib;

FIG. 29B illustrates a transcript diagram illustrating alternative splicing of MCL1 to generate MCL1-short (S, pro-apoptosis) and MCL1-long (L, anti-apoptosis) variants. For human cells, the ratio of MCL1-short to long isoforms is shown;

FIG. 29C illustrates MCL1 S/L ratios in Rebecsinib dose response assays performed using primary sAML LSC (without stromal co-culture);

FIG. 29D illustrates a schematic diagram outlining multispecies toxicokinetic (TK) and pharmacodynamic studies in mammalian species treated in vivo with a single dose of Rebecsinib;

FIG. 29E-F illustrate toxicokinetic analyses, rats (FIG. 29E) and rabbits (FIG. 29F) were given a single injection of Rebecsinib at 1-40 mg/kg, or vehicle control, and blood samples were drawn at regular intervals to determine plasma concentrations of the compound over 8 hr after treatment; and

FIG. 29G-H illustrate in vivo TK and complementary pharmacodynamic studies where NHPs were given a single injection of Rebecsinib at 3-20 mg/kg, or vehicle control, and blood samples were drawn at regular intervals to determine plasma concentrations (FIG. 29G) of the compound along with splice isoform biomarker assays to quantify MCL1 exon skipping in PBMCs isolated from treated animals (FIG. 29H),

as discussed in further detail in Example 14, below.

FIG. 30A-H illustrate ADAR1 expression and LSC self-renewal following Rebecsinib treatment:

FIG. 30A illustrates a schematic diagram showing in vivo treatment of primary patient LSC or cord blood (CB)-engrafted mice and sAML serial transplantation studies;

FIG. 30B illustrates a flow cytometry analysis quantifying human LSC survival in sAML-engrafted mice treated with Rebecsinib Lot 1 or Lot 2 compared with vehicle control at 10 mg/kg twice weekly for two weeks;

FIG. 30C illustrates a qRT-PCR analyses in CD34+ cells isolated from the spleens of sAML50261 mice treated with Rebecsinib (as in A) showing decreased total ADAR1 expression by qRT-PCR;

FIG. 30D illustrates a mean fluorescence intensity (MFI) of ADAR1p150 protein levels in human HSCs (CD45⁺CD34⁺CD38⁻Lin⁻) and HPCs (CD45⁺CD34⁺CD38⁺Lin⁻) from the spleens of sAML50261 engrafted mice treated with Rebecsinib or vehicle;

FIG. 30E illustrates a whole transcriptome-based RNA editing analyses of previously-described RNA-seq data¹⁴ generated from CD34⁺ cells isolated from the spleens of sAML50261 engrafted mice treated with Rebecsinib or vehicle;

FIG. 30F illustrates isoform-level analysis of MCL1 transcripts from RNA-sequencing data shown in FIG. 30E;

FIG. 30G illustrates overall mouse survival in serially transplanted sAML mice (primary transplanted mice were treated with Rebecsinib or vehicle); and

FIG. 30H illustrates ratios of ADAR1p150-3′UTR truncated (ADAR-208) to ADAR1p110 (ADAR-201) by RNA-seq analyses of CD34⁺ cells isolated from serial transplant recipients of sAML50261 LSC engrafted mice treated with vehicle or Rebecsinib,

as discussed in further detail in Example 14, below.

Like reference symbols in the various drawings indicate like elements.

### DETAILED DESCRIPTION

In alternative embodiments, provided are methods for promoting expansion of human CD34⁺ cells in vivo and sparing CD45⁺ cell survival in vivo, promoting normal hematopoietic stem cell retention in the bone marrow niche, or for treating, preventing or ameliorating a spinal cord injury, liver cirrhosis and peripheral vascular disease, and for promoting neuronal regeneration. In alternative embodiments, provided are methods for treating, preventing or ameliorating a JAK2-related disease or a ADAR-related disease, or a cancer, neoplasm or tumor, comprising administering to an individual in need thereof a drug combination comprising rebecsinib and fedratinib.

In alternative embodiments, provided are methods comprising use of a therapeutic combination of fedratinib and rebecsinib, including formulations, pharmaceutical compositions and products of manufacture (such as medical devices, or implant) comprising fedratinib and rebecsinib, and methods of using them.

In alternative embodiments, the therapeutic combination of drugs as provided herein comprising fedratinib and rebecsinib can modulate the spliceosome, can down-regulate levels of ADAR enzymes, and can be used to treat, ameliorate, slow the progression of and/or prevent (used as a prophylactic treatment for) JAK2-related diseases, ADAR-related diseases, or both, including diseases and conditions such as neoplasms, tumors and cancers, including cancers lacking receptors for estrogen, progesterone, and HER2 (human epidermal growth factor receptor 2; or CD340 (cluster of differentiation 340)), such as triple negative breast cancer. In alternative embodiments, the term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

In alternative embodiments, the term “composition” refers to a mixture of at least two or more components, for example, comprising fedratinib and rebecsinib.

In alternative embodiments, provided are methods for administering to an individual in need thereof an effective amount and/or a therapeutically effective amount of a therapeutic combination of drugs as provided herein comprising fedratinib and rebecsinib. In alternative embodiments, the terms “effective amount” and “therapeutically effective amount” refer to an amount of therapeutic compound, combination of compounds, or composition, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

In alternative embodiments, the therapeutic combination of drugs as provided herein comprising fedratinib and rebecsinib are administered to an individual in need thereof in the form or (or formulated as) a pharmaceutically acceptable carrier, and in alternative embodiments a “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound (or fedratinib and rebecsinib as described herein) within or to the patient such that the compound or therapeutic combination of drugs may perform its intended function. In alternative embodiments a given carrier is “acceptable” in the sense of being compatible with the other ingredients of a particular formulation, including the compounds described herein, and not injurious to the patient (or the individual in need thereof).

In alternative embodiments other pharmaceutically active, or non-active, ingredients are included in the pharmaceutical compositions described herein, including for example additional ingredients known in the art and described, for example, in “Remington's Pharmaceutical Sciences” (Genaro (Ed.), Mack Publishing Co., 1985), the entire content of which is incorporated herein by reference.

In alternative embodiments, the therapeutic combination of drugs as provided herein comprising fedratinib and rebecsinib are each separately or together formulated as a pharmaceutically acceptable salt. In alternative embodiments, the term “pharmaceutically acceptable salt” refers to derivatives of the disclosed compounds wherein one or both compounds are modified by converting an existing acid or base moiety to its salt form. Pharmaceutically acceptable salts can be synthesized from the parent compound which contains a basic or acidic moiety by conventional chemical methods. Such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

In alternative embodiments, the term “pharmaceutical composition” refers to a mixture of at least one compound described herein with a pharmaceutically acceptable carrier. The pharmaceutical composition facilitates administration of the compound, or combination thereof, to a patient or subject or individual in need thereof. Multiple techniques of administering a compound, combination, or composition, exist including, but not limited to, intravenous, oral, aerosol, parenteral, ophthalmic, pulmonary, and topical administration. In alternative embodiments, the fedratinib and rebecsinib are formulated separately and administered to a patient or subject or individual in need thereof substantially at the same time, or the fedratinib and rebecsinib are formulated together.

In alternative embodiments, the terms “treatment” or “treating” refer to the application of one or more specific procedures used for the amelioration of a disease, for example, a cancer. In alternative embodiments, a “prophylactic” treatment refers to reducing the rate of progression of the disease or condition (such as cancer) being treated, delaying the onset of that disease or condition, or reducing the severity of its onset.

### Therapeutic Combinations Comprising Rebecsinib and Fedratinib

In alternative embodiments, provided are methods for using formulations and therapeutic combinations comprising rebecsinib and fedratinib. Data presented herein demonstrates that formulations and therapeutic combinations comprising rebecsinib and fedratinib are useful in treating, preventing or ameliorating a spinal cord injury, liver cirrhosis and peripheral vascular disease, and for promoting neuronal regeneration.

Formulations and a pharmaceutical combination of rebecsinib and fedratinib are described in WO2022165398A1 and U.S. patent application publication no. US2024/0148688A1, which are incorporated herein by reference.

Rebecsinib has a structure as shown below, see WO 2021/026273 A1 and U.S. Pat. No. 10,675,267 B2, which are incorporated herein by reference. Rebecsinib has also been synthesized as described by Chan et al. (Cell Reports Physical Science, 2020, 1, 12, 100277), and U.S. patent application publication no. US2024/0148688A1.

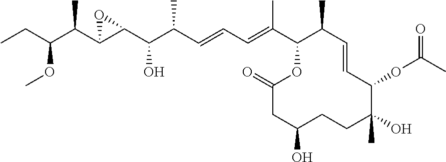 [SMILES: CC[C@H](OC)[C@@H](C)[C@H]1O[C@@H]1[C@@H](O)[C@H](C)/C=C/C=C(\C)[C@H]1OC(=O)C[C@H](O)CC[C@@](C)(O)[C@@H](OC(C)=O)/C=C/[C@@H]1C]

In alternative embodiments rebecsinib is provided a neutral form or as a hydrate.

Fedratinib is an approved therapeutic having a structure shown below. Fedratinib has been described in U.S. Pat. Nos. 7,528,143 B2, 7,825,246 B2, 8,138,199 B2, 10,391,094 B2, and 11,400,092 B2, which are incorporated herein by reference. In alternative embodiments fedratinib is provided in a neutral form, or as a pharmaceutically acceptable salt, or as a hydrate, or a di-salt, for example, as a dihydrochloride salt. In some embodiments, fedratinib may be in the form of a dihydrochloride monohydrate (N-tert-butyl-3-[(5-methyl2-{[4-(2-pyrrolidin-1-ylethoxy)phenyl]amino}pyrimidin-4-yl)amino]benzenesulfonamide dihydrochloride monohydrate).

### Fedratinib:

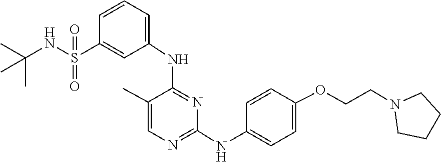 [SMILES: Cc1cnc(Nc2ccc(OCCN3CCCC3)cc2)nc1Nc1cccc(S(=O)(=O)NC(C)(C)C)c1]

Rebecsinib and fedratinib compounds described herein may be provided as isotopically-labeled compounds wherein one or more atoms, independently, may be replaced by an atom having the same atomic number, but an atomic mass or mass number different from the atomic mass or mass number predominantly found in nature. Examples of isotopes suitable for inclusion in the compounds described herein include, but are not limited to, ²H, ³H, ¹¹C, ¹³C, ¹⁴C, ³⁶Cl, ¹³N, ¹⁵N, ¹⁵O, ¹⁷O, ¹⁸O or ³⁵S. In some embodiments, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁵O, or ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as described herein are modified by the use of appropriate reagents and conditions, for the introduction of the various moieties found in the formula as provided herein.

Compounds described herein are synthesized using any suitable procedures starting from compounds that are available from commercial sources, including procedures described in the above-identified patent documents.

### Formulations

In alternative embodiments, fedratinib and rebecsinib combinations as provided herein are a combination of the two in a single composition, or a combination of the two in separate compositions, which may be administered simultaneously or sequentially.

In some embodiments, the fedratinib and rebecsinib is formulated as a pharmaceutical composition. In some embodiments, the pharmaceutical compositions referred to herein may include at least one pharmaceutically acceptable carrier.

In some embodiments, provided herein are packaged compounds, packaged compositions, or packaged pharmaceutical compositions, comprising a container holding a therapeutically effective amount of rebecsinib, fedratinib, or both, and instructions for using the compound(s) in accordance with one or more of the methods provided herein.

The present combination and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, exemplary fedratinib and rebecsinib combinations can also be provided in kit form combined with other components necessary for administration of the material to the patient. For example, disclosed kits, such as for use in the treatment of cancer, can further comprise, for example, administration materials. The kits are designed in various forms based on the specific deficiencies they are designed to treat.

In alternative embodiments the compounds, combinations, or compositions provided herein may be prepared and placed in a container for storage at ambient or elevated temperature. When the compound, combination, or composition is stored in a polyolefin plastic container as compared to a polyvinyl chloride plastic container, discoloration of the compound or composition, or sorption of the compound with the surface of the container, may be reduced, whether dissolved or suspended in a liquid composition (for example, an aqueous or organic liquid solution), or as a solid. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce exposure of the container's contents to infrared light, or also include a second component with such a capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. The compounds, combinations, or compositions provided herein may need storage lasting up to, or longer than, three months; in some cases up to, or longer than one year. The containers may be in any form suitable to contain the contents; for example, a bag, a bottle, or a box.

### Methods of Use

In alternative embodiments, provided are methods for promoting expansion of human CD34⁺ cells in vivo and sparing CD45⁺ cell survival in vivo, or for treating, preventing or ameliorating a spinal cord injury, liver cirrhosis and peripheral vascular disease, and for promoting neuronal regeneration. In alternative embodiments, provided are methods for treating, preventing or ameliorating a JAK2-related disease or a ADAR-related disease, or a cancer, neoplasm or tumor, comprising administering to an individual in need thereof a drug combination comprising rebecsinib and fedratinib.

In some embodiments, provided herein are methods of inhibiting mRNA splicing activity, or down-regulating ADAR levels, in a subject in need thereof, comprising administering fedratinib and rebecsinib to the subject.

The methods described herein may occur in vivo or in vitro, including within a subject, such as a human subject. In some embodiments, the methods are applied to a cell in vitro. In some embodiments, the methods are applied to a cell in vivo, for example, applied to a subject such as a mammalian subject or a human subject.

In alternative embodiments actual dosage levels of the active ingredients of the combinations described herein, rebecsinib and fedratinib, may be independently varied so as to obtain amounts of the active ingredients effective to achieve the desired therapeutic response for a particular patient, composition, and mode of administration, without being toxic to the patient.

In alternative embodiments, the selected dosage levels will depend upon a variety of factors including the activity of the particular combination employed, the time of administration, the rate of excretion of the compounds in the combination, the duration of the treatment, other drugs, compounds or materials used in further combination with the rebecsinib and fedratinib combination, the age, sex, weight, condition, general health, and prior medical history of the patient being treated, and like factors well-known in the medical arts. A medical doctor, e.g., physician or veterinarian, having ordinary skill in the art may readily determine and prescribe the effective amount of the pharmaceutical composition(s) required. For example, the physician or veterinarian could start doses of the compounds employed in the pharmaceutical composition(s) at levels lower than that required in order to achieve the desired therapeutic effect and gradually increase the dosage until the desired effect is achieved.

In alternative embodiments routes of administration of the combinations herein include, without limitation, oral, nasal, rectal, intravaginal, parenteral, buccal, sublingual, or topical. In some embodiments, the oral or nasal route of administration is an oral inhalational or nasal inhalational route of administration. The compounds for use as described herein may be formulated for administration by any suitable route to achieve the particular method being applied.

### Formulations and Pharmaceutical Compositions

In alternative embodiments, provided are pharmaceutical formulations or compositions comprising drugs, and therapeutic combinations of drugs, and formulations, and liposomes, for practicing methods and uses as provided herein, including methods for promoting expansion of human CD34⁺ cells in vivo and sparing CD45⁺ cell survival in vivo, promoting normal hematopoietic stem cell retention in the bone marrow niche, or for treating, preventing or ameliorating a spinal cord injury, liver cirrhosis and peripheral vascular disease, and for promoting neuronal regeneration. In alternative embodiments, provided are methods for treating, preventing or ameliorating a JAK2-related disease or a ADAR-related disease, or a cancer, neoplasm or tumor, comprising administering to an individual in need thereof a drug combination comprising rebecsinib and fedratinib

In alternative embodiments, a formulation or pharmaceutical compositions used to practice methods and uses as provided herein can be administered parenterally, topically, orally or by local administration, such as by aerosol or transdermally, or intravitreal injection. The formulations and pharmaceutical compositions (including therapeutic drug combinations) can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, these compositions used to practice methods and uses as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods or uses as provided herein can be in forms such as tablets, pills, powders, capsules, liquids, gels, syrups, slurries, suspensions, etc., suitable for in vivo, in vitro or ex vivo applications.

In alternative embodiments, formulations and pharmaceutical compositions used to practice methods and uses as provided herein can comprise a solution of compositions (for example, any active agent as used in methods provided herein) disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice methods and uses as provided herein are sterile and can be manufactured to be generally free of undesirable matter. In one embodiment, these solutions and formulations are sterilized by conventional, well known sterilization techniques.

The solutions and formulations used to practice methods and uses as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compositions and formulations used to practice methods and uses as provided herein can be delivered by the use of liposomes. By using liposomes, particularly where the liposome surface carries ligands specific for target cells (for example, an injured or diseased neuronal cell or CNS tissue), or are otherwise preferentially directed to a specific tissue or organ type, one can focus the delivery of the active agent into a target cells in an in vivo, in vitro or ex vivo application.

### Nanoparticles, Nanolipoparticles and Liposomes

Also provided are nanoparticles, nanolipoparticles, vesicles and liposomal membranes comprising compounds used to practice methods and uses as provided herein, for example, to deliver compositions used to practice methods as provided herein, for example, to deliver a drug or drugs, for example to practice a method as provided herein.

Provided are multilayered liposomes comprising compounds used to practice methods and uses as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods and uses as provided herein.

Liposomes can be made using any method, for example, as described in Park, et al., U.S. Pat. Pub. No. 20070042031, including method of producing a liposome by encapsulating an active agent (for example, a drug combination as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods and uses as provided herein comprise a substituted ammonium and/or polyanions, for example, for targeting delivery of a compound (for example, a drug or drug combination as provided herein) to a desired cell type (for example, a neural cell, or a cancer cell), as described for example, in U.S. Pat. Pub. No. 20070110798.

Provided are nanoparticles comprising compounds (for example, a drug or drug combination as provided herein) in the form of active agent-containing nanoparticles (for example, a secondary nanoparticle), as described, for example, in U.S. Pat. Pub. No. 20070077286. In one embodiment, provided are nanoparticles comprising a fat-soluble active agent or a fat-solubilized water-soluble active agent to act with a bivalent or trivalent metal salt.

In one embodiment, solid lipid suspensions can be used to formulate and to deliver compositions used to practice methods and uses as provided herein to mammalian cells in vivo, for example, to the CNS, as described, for example, in U.S. Pat. Pub. No. 20050136121.

### Delivery Cells and Delivery Vehicles

In alternative embodiments, any delivery vehicle can be used to practice the methods or uses as provided herein, for example, to deliver compositions (for example, a drug or drug combination as provided herein) in vivo, to an individual in need thereof. For example, delivery vehicles comprising polycations, cationic polymers and/or cationic peptides, such as polyethyleneimine derivatives, can be used for example as described, for example, in U.S. Pat. Pub. No. 20060083737.

In one embodiment, a dried polypeptide-surfactant complex is used to formulate a composition used to practice methods as provided herein, for example as described, for example, in U.S. Pat. Pub. No. 20040151766.

In one embodiment, a composition used to practice methods and uses as provided herein can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In alternative embodiments, a drug or drug combination as provided herein is delivered in vivo using methods as provided herein formulated in a lipid formulation or a liposome and injected for example intramuscularly (IM), for example using formulations and methods as described in U.S. patent application no. US 20210046173 A1; wherein optionally the drug or drugs is/are formulated in a liposome, or a lipid nanoparticle (LNP), or nanoliposome, that comprises: non-cationic lipids comprise a mixture of cholesterol and DSPC, or a PEG-lipid, or PEG-modified lipid, or LNP, or an ionizable cationic lipid; or a mixture of (13Z,16Z)-N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9 Z)-octadec-9-en-1-yloxy]propan-2-amine; (13Z,16Z)-N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (13Z,16Z)-N,N-dimethyl-3-nonyldocosa-13,16-dien-1-amine, (12Z,15Z)-N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)-N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Dosaging

The pharmaceutical compositions, drug combinations and formulations used to practice methods and uses as provided herein can be administered for prophylactic and/or therapeutic treatments as provided herein.

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The pharmaceutical compositions, drug combinations and formulations used to practice methods and uses as provided herein can be administered as a single dosage or in multiple dosages, as needed. In alternative embodiments, these dosages are administered intravitreally, orally, IM, IV, or intrathecally. In alternative embodiments, the vectors are delivered as formulations or pharmaceutical preparations, for example, where the drug or drugs are contained in a nanoparticle, a particle, a micelle or a liposome or lipoplex, a polymersome, a polyplex or a dendrimer. In alternative embodiments, these dosages are administered once a day, once a week, or any variation thereof as needed to maintain in vivo expression levels of a desired drug, which can be monitored by assessing the therapeutic effect, for example, to treat, ameliorate, protect against, reverse or decrease the severity or duration of a cancer. The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations, therapeutic drug combinations can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice methods as provided herein are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Reversal of Malignant ADAR1 Splicing Promotes Normal Hematopoietic Stem Cell Retention and Prevents Leukemia Stem Cell Propagation

This example demonstrates exemplary methods for treating and diagnosing cancer.

Acute myeloid leukemia (AML) is characterized by a clonal proliferation of malignant myeloid precursors that harbor a reduced capacity for differentiation. Adenosine deaminase acting on RNA 1 (ADAR1) is an RNA editing enzyme that catalyzes the conversion of adenosine bases to inosine, which can alter protein function. ADAR1 has been shown to drive cancer stem cell (CSC) generation and therapeutic resistance in multiple malignancies^{1, 2}. Treatment with 17S-FD-895 (rebecsinib), a selective small molecule splicing modulator that targets the splicing factor 3b subunit 1 (SF3B1) component of the spliceosome, has been shown to reverse malignant splice isoform switching, inhibit activation of the interferon-inducible ADAR1p150 splice isoform, and significantly reduce leukemia stem cell (LSC) maintenance in vitro and in preclinical patient-derived AML mouse models^{3,4} However, the effectiveness of rebecsinib versus current therapies for the treatment of AML and the effects of rebecsinib treatment on aged normal bone marrow (aNBM) stem cells has not yet been elucidated.

Here, we established humanized mouse models using CD34⁺ stem cells isolated from the peripheral blood of adult secondary AML (sAML) patients or bone marrow of normal-aged donors (63-79 years old). Strikingly, sAML-engrafted mice treated with rebecsinib survived significantly longer compared to those treated with vehicle, fedratinib (an FDA-approved JAK2 inhibitor), and the combination of rebecsinib and fedratinib. Interestingly, aged normal bone marrow engrafted mice treated with rebecsinib (10 mg/kg, biweekly for 2 weeks) had an increased number of human CD34⁺ cells in their bone marrow.

Collectively, this data demonstrates that inhibition of ADAR1p150 may provide a competitive advantage for normal hematopoietic stem cells compared with leukemia stem cells in the bone marrow. These findings lay a foundation for developing rebecsinib as a clinical ADAR1 antagonist that promotes normal hematopoietic stem cell retention in the bone marrow niche.

### Figure Legends

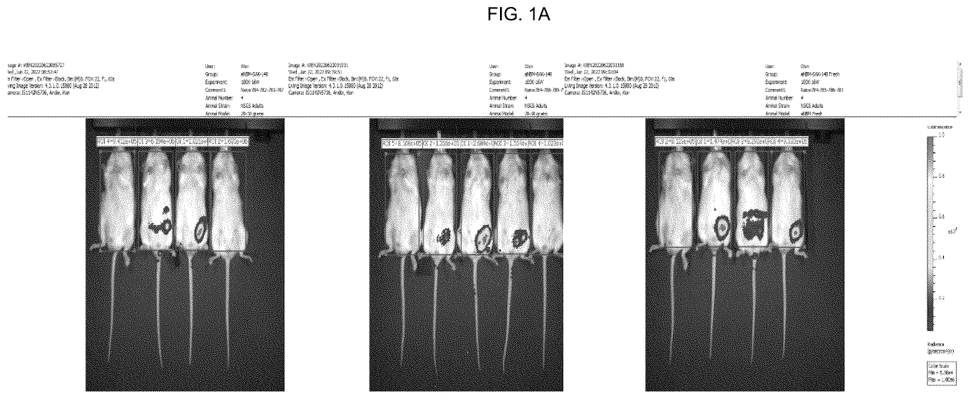

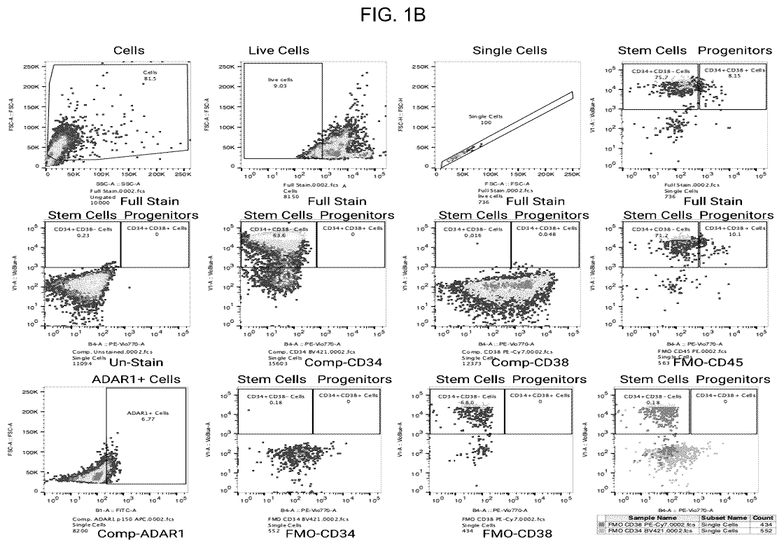

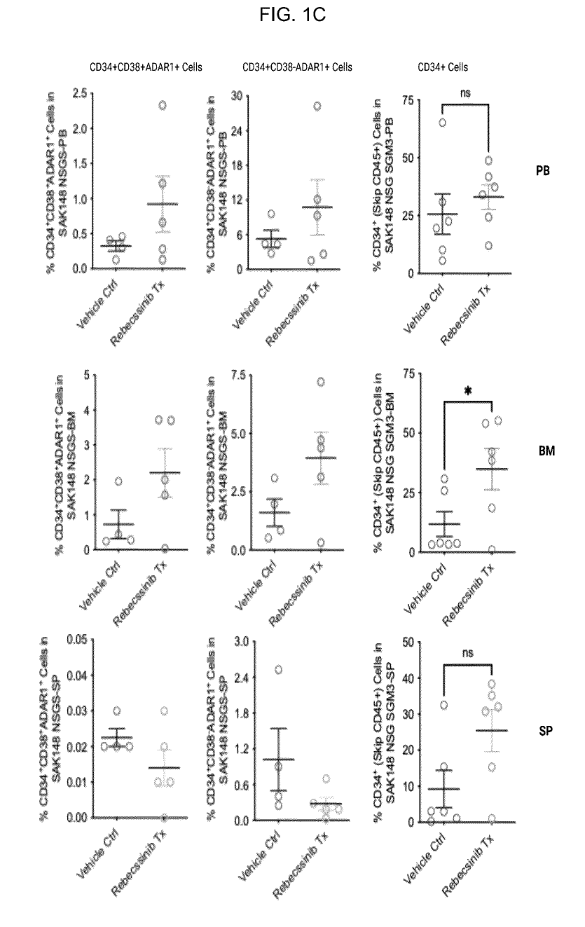

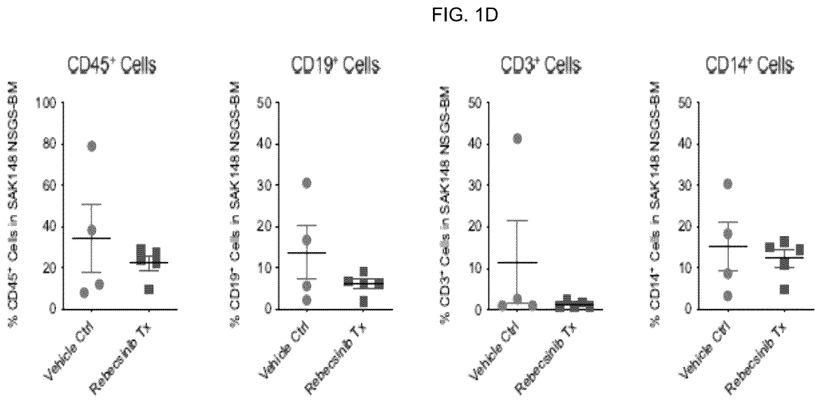

FIG. 1: Rebecsinib treatment promotes human CD34⁺ cells and spares CD45⁺ cells Aged normal bone marrow (BM) samples were provided by Scripps Clinic La Jolla, CA. Normal human CD34⁺ cells were freshly isolated with Microbeads (Miltenyi Biotech) and subsequently transduced with the ADAR1-NanoLuc reporter for 48 hours. Adult NSG-SGM3 mice (4-6 weeks) were intravenously transplanted with 100K ADAR1-NanoLuc reporter transduced CD34⁺ cells. The mice were screened using the IVIS 200 imaging system and staining of peripheral blood with human CD45 monoclonal antibody. Mice with human CD45⁺ cells >1% were included in the treatment cohort receiving Rebecsinib at a dose of 10 mg/kg, intravenously, twice a week for 2 weeks, with a total of 5 doses. After treatment, mice were sacrificed, and single cells were obtained from peripheral blood (PB), bone marrow (BM), and spleen (SP) for further analysis. A. IVIS 200 images of NSG-SGM3 mice intravenously transplanted with ADAR1-NanoLuc reporter transduced normal human CD34⁺ cells at 18 weeks after transplantation. B. Flow cytometry gating strategies showing positive and negative controls. C. Treatment with Rebecsinib, a selective small molecule splicing modulator, leads to increased levels of human CD34⁺ cells in PB, SP, and a significant CD34⁺ increase in BM cells (p=0.046, Student's t-test). D. Rebecsinib treatment preserves human CD45⁺ cells and differentiated cells, including CD14⁺ myeloid cells, and lymphoid cells such as CD3⁺ T and CD19⁺ B cells.

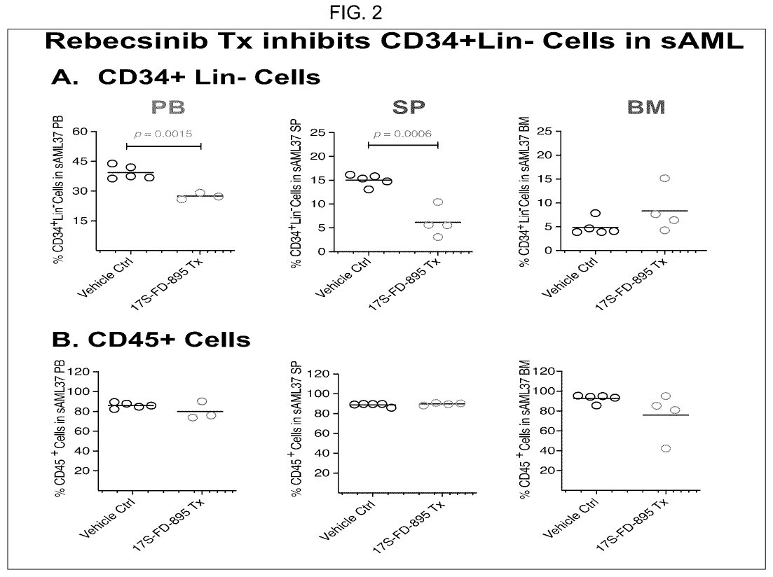

FIG. 2: Rebecsinib treatment inhibits CD34⁺Lin⁻ cells in sAML PDX models To establish a humanized sAML mouse model, neonatal Rag2^{-/-}gc^{-/-} immunocompromised mice were intrahepatically transplanted with 100K human CD34⁺ cells isolated from the sAML50261 patient. The mouse models were screened by staining the PB of each mouse with a human CD45 mAb. Mice with a threshold of human CD45⁺ cells >1% were included in the treatment cohort for Rebecchini. The dosing plan involved administering Rebecsinib at a dose of 10 mg/kg, intravenously, twice a week for 2 weeks, with a total of 5 doses. Single cells were isolated from the mouse organs, including PB, SP, and BM, and subjected to staining with human stem cell markers and lineage cell markers. The results demonstrated the following: A. The CD34⁺Lin⁻ cell population exhibited significant inhibition in both PB and SP (p=0.0015 and p=0.0006, respectively, Student's t-test). B. Rebecsinib treatment preserved human CD45⁺ cells in the organs of PB, BM, and SP.

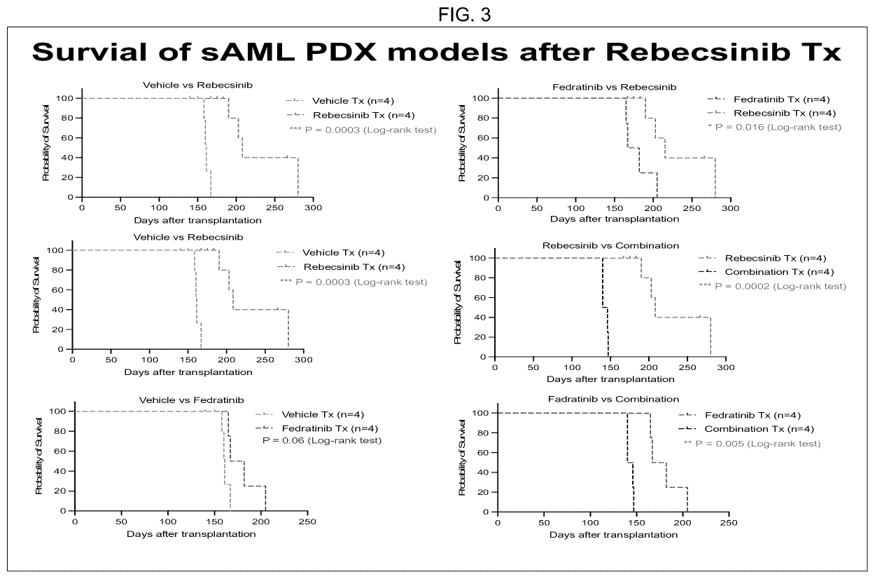

FIG. 3: Rebecsinib treatment significantly increases sAML PDX survival The successful sAML mouse models were randomly grouped into 4 treatment conditions including (1) vehicle treatment, (2) single drug treatment with Fedratinib, (3) single drug treatment with Rebecsinib, and (4) combination treatment with both drugs. These mice were monitored for survival throughout the study until they reached the endpoint. Survival data were analyzed using a Log-rank test.

The results revealed the following: The humanized sAML mice treated with the combination of fedratinib and rebecsinib exhibited a significantly longer survival time compared to those treated with single fedratinib (p=0.005, Log-rank test) or single rebecsinib (p=0.0002, Log-rank test). Interestingly, the survival time of mice treated with single rebecsinib was significantly longer than those treated with single fedratinib (p=0.016, Log-rank test) and vehicle (p=0.0003, Log-rank test).

### Conclusions

1. The observation that rebecsinib treatment increased human CD34⁺ cells in the bone marrow of aNBM-engrafted mice suggests that inhibiting ADAR1p150 may confer a competitive advantage to normal hematopoietic stem cells (HSCs) over leukemic stem cells (LSCs) within the bone marrow microenvironment.
2. Rebecsinib treatment shows promise in potentially enhancing the efficacy of AML treatment by promoting the retention of normal HSCs in the BM niche.
3. Preclinical PDX AML mouse models demonstrated that Rebecsinib treatment can reduce the maintenance of LSCs.
4. In sAML-engrafted mice, treatment with Rebecsinib demonstrated superior efficacy in promoting survival compared to Fedratinib or vehicle treatment alone.

### Example 2: Role of RNA and DNA Editing in Normal Hematopoietic Stem and Progenitor Cell Maintenance and Malignant Transformation in Myeloproliferative Neoplasms

This example demonstrates exemplary methods for treating and diagnosing cancer.

Dysregulation of inflammatory cytokine responsive APOBEC3 cytosine deaminases has been shown to be a contributing factor in cancer evolution, presenting as gene expression changes and inclusion of distinct C-to-T mutation patterns. However, the context specificity and mechanisms by which APOBEC3 enzymes regulate hematopoietic stem cell function and the role they play in cancer initiation and progression require further elucidation. Lentiviral overexpression of APOBEC3C and an editase deficient APOBEC3C mutant in healthy cord blood, bone marrow and myeloproliferative neoplasm patient hematopoietic stem/progenitor cells (HSPCs) allows us to study the effects of innate immune deaminase dysregulation in the hematopoietic niche. By FACS sorting individual stem and progenitor cell populations, we can examine the distinct role of RNA and DNA editing in both normal hemopoietic stem cell fate determination as well as malignant transformation into cancer stem cells.

We are focusing on the upregulation of APOBEC3C and adenosine deaminase acting on RNA1 (ADAR1), as we have previously shown them to be contemporaneously upregulated in the high-risk myelofibrosis (MF) stem cell population compared to normal aged bone marrow. By performing whole genome and whole transcriptome analysis we can compare these novel differential gene expression changes, RNA hyper-editing sites, and DNA mutation signatures induced by APOBEC3 mutagenesis to abnormalities seen in both hematopoietic malignancies and solid tumor cancers. Gene set enrichment analysis (GSEA) performed on this dataset has exposed numerous deregulated pathways brought on by exaggerated levels of APOBEC3, including changes in splicing pathways. Long term, we aim to use these findings to identify predictive biomarkers and druggable targets of leukemic initiation and progression.

Apolipoprotein B mRNA editing enzyme catalytic polypeptide-like APOBEC is a family of cytidine deaminases that catalyze the conversion of cytosine to uracil (read as Thymidine) creating a functional C to T conversion.

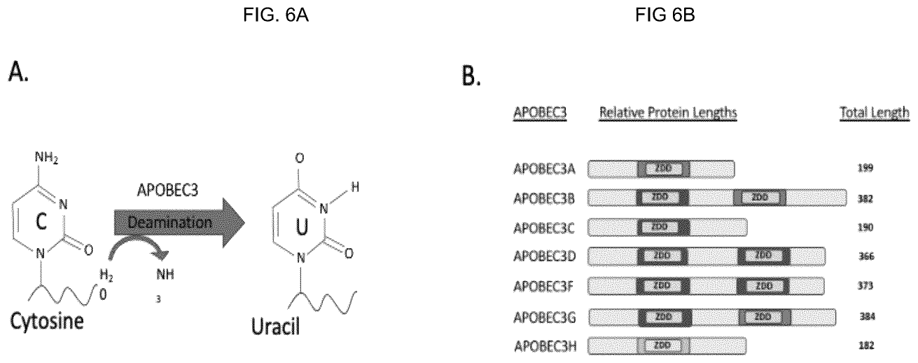

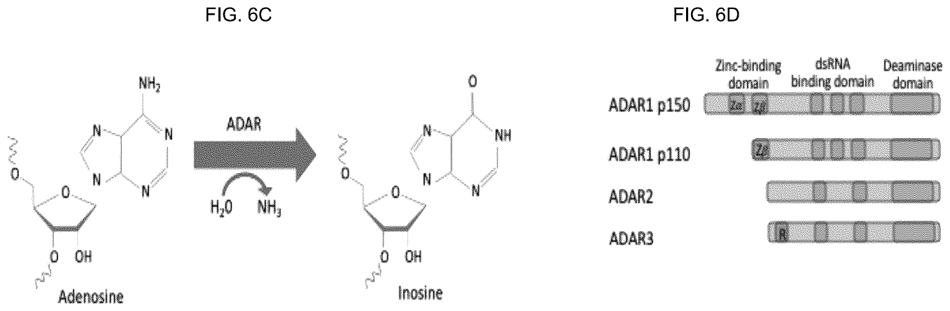

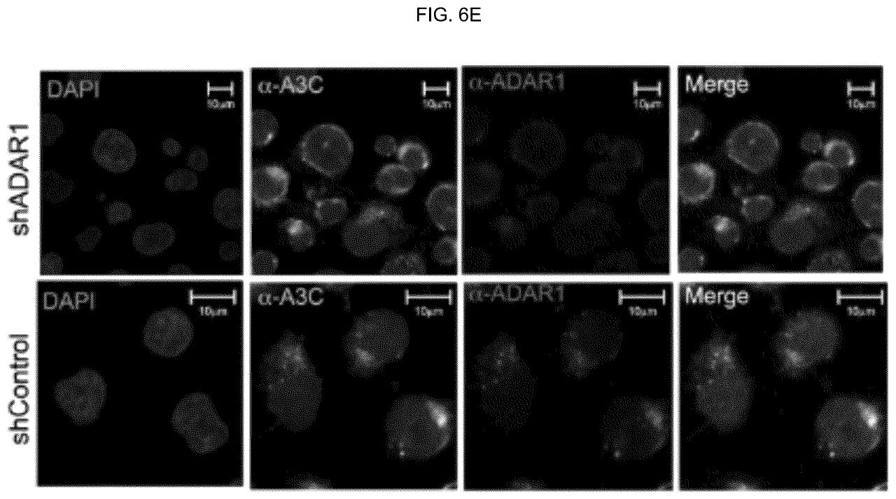

FIG. 6A-E: Innate immune deaminases: APOBEC3 and ADAR1; FIG. 6E: Colocalization of APOBEC3C and ADAR1 in TF1a cells. Immunofluorescence of anti-APOBEC3C (green) and anti-ADAR1 p150-specific (red) antibodies in TF1a shADAR1 and TF1a shControl knockdown cells demonstrate a colocalization (yellow) of APOBEC3C and ADAR1 p150 proteins in the shControl cells. TF1a shADAR1 cells show ablation of ADAR1 protein.

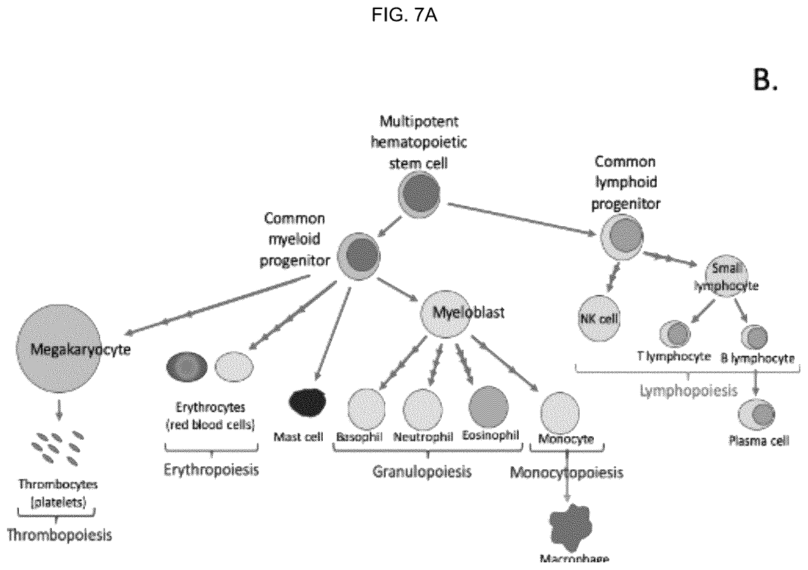

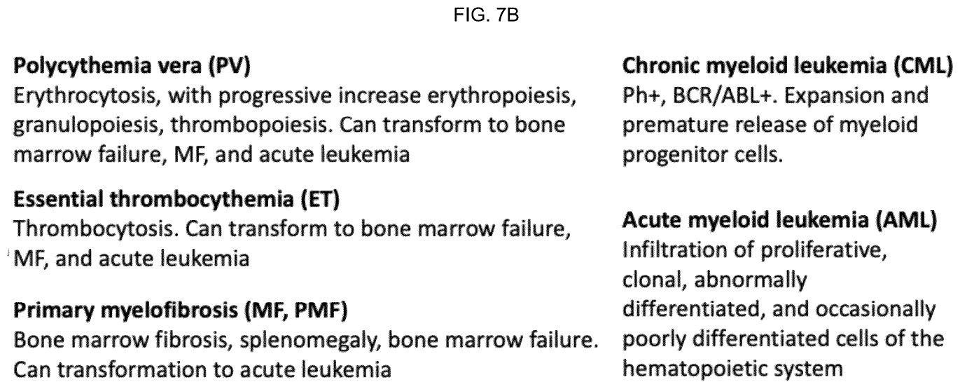

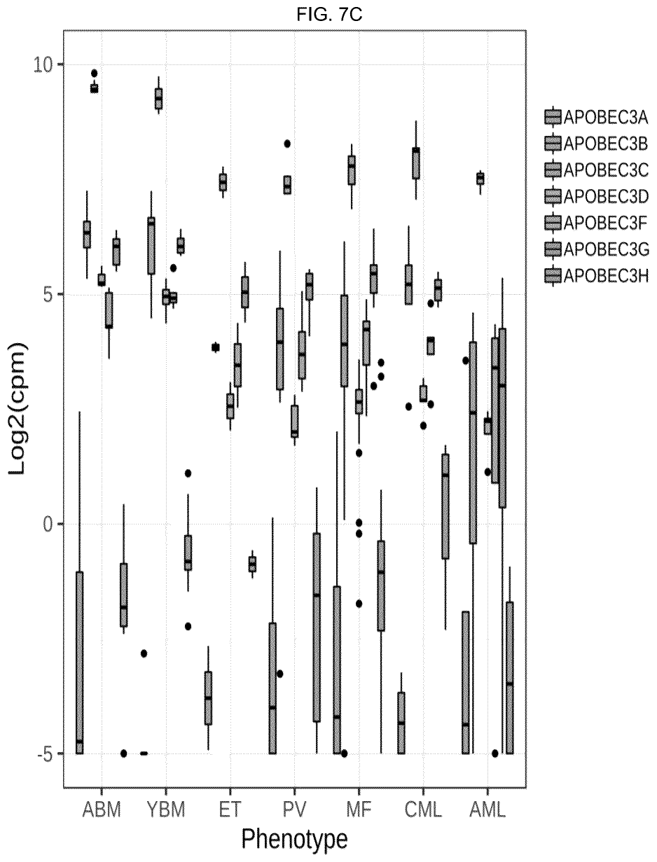

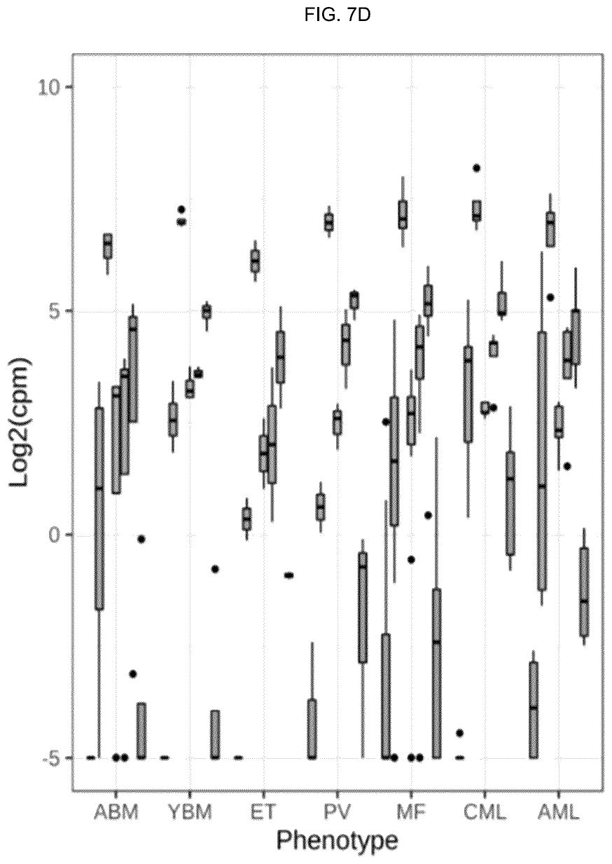

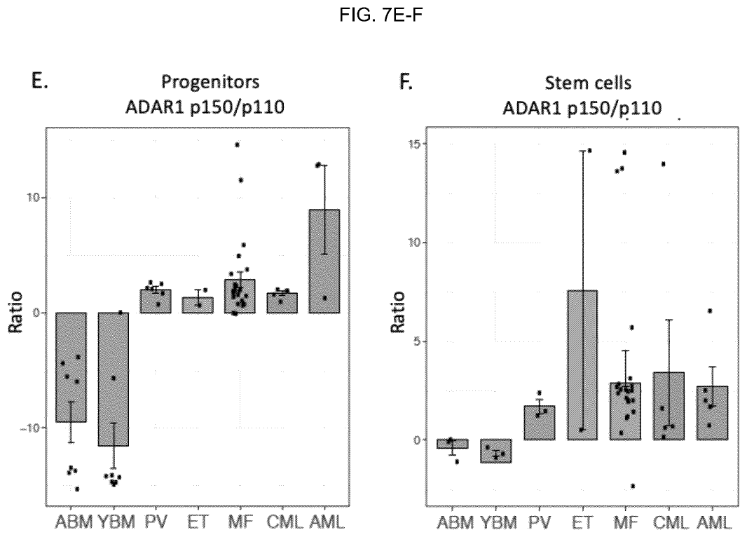

FIG. 7 A-F, Differential expression of APOBEC3 and ADAR1 in hematopoietic malignancies: Differential expression of APOBEC3s in multiple disease states as compared to young and aged normal peripheral blood. A. Normal hematopoiesis. B. Clinical characterization of myeloproliferative neoplasms. C. APOBEC3 expression in progenitor cell populations in normal young and aged samples and across the myeloproliferative neoplasm disease spectrum. Notably APOBEC3C (green) shows the highest expression of all the APOBEC3 genes in both progenitor and stem cell populations. D. APOBEC3C expression in the stem cell populations of normal young and aged samples and myeloproliferative neoplasms. E. ADAR1 expression in the stem population of aged and young bone marrow, and in myeloproliferative neoplasm patient samples. F. ADAR1 expression in the stem population of young and aged normal controls, and in myeloproliferative neoplasm patient samples.

FIG. 7B: Polycythemia vera (PV)

Erythrocytosis, with progressive increase erythropoiesis, granulopoiesis, thrombopoiesis can transform to bone marrow failure, MF, and acute leukemia. Essential thrombocythemia (ET) Thrombocytosis can transform to bone marrow failure, MF, and acute leukemia Primary myelofibrosis (MF, PMF) Bone marrow fibrosis, splenomegaly, bone marrow failure can transform to acute leukemia. Chronic myeloid leukemia (CML) Ph+, BCR/ABL+. Expansion and premature release of myeloid progenitor cells. Acute myeloid leukemia (AML) Infiltration of proliferative, clonal, abnormally differentiated, and occasionally poorly differentiated cells of the hematopoietic system.

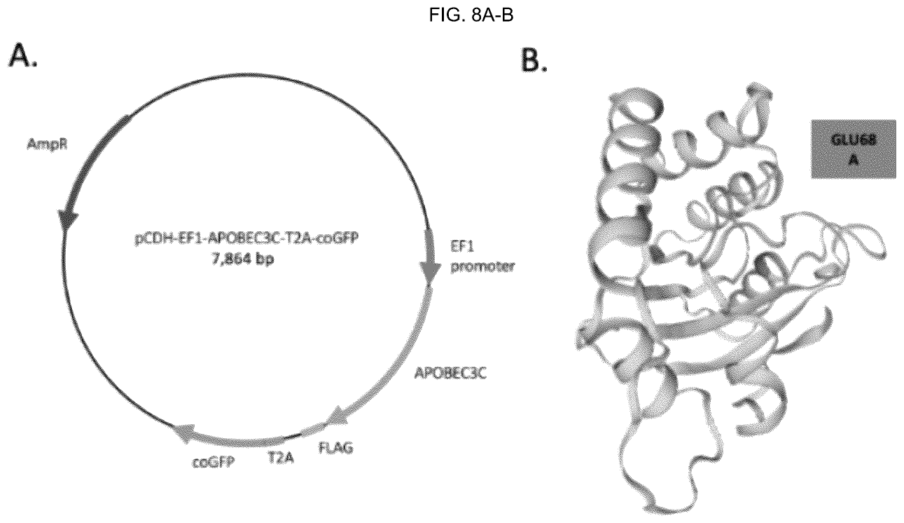

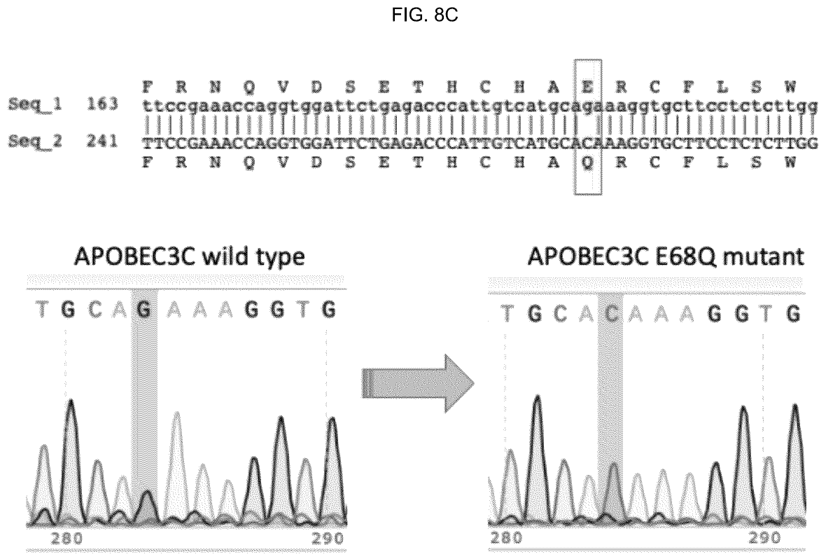

FIG. 8 A-C: APOBEC3 lentiviral overexpression: Construction and modification of APOBEC3C plasmids. A. Vector map of APOBEC3C lentiviral vector. FLAG-tags were added to constructs for use in pull-down experiments. B. Crystal Structure of APOBEC3C, with the active site, Glu68 highlighted. From Swiss-Model, University of Basel Center for Molecular Life Sciences. C. APOBEC3C E68Q vector was created using site-directed mutagenesis and introducing a g202c point mutation. This mutation functionally replaces Glutamic acid (Glu) 68 by Glutamine (Gln) and renders APOBEC3C catalytically inactive.

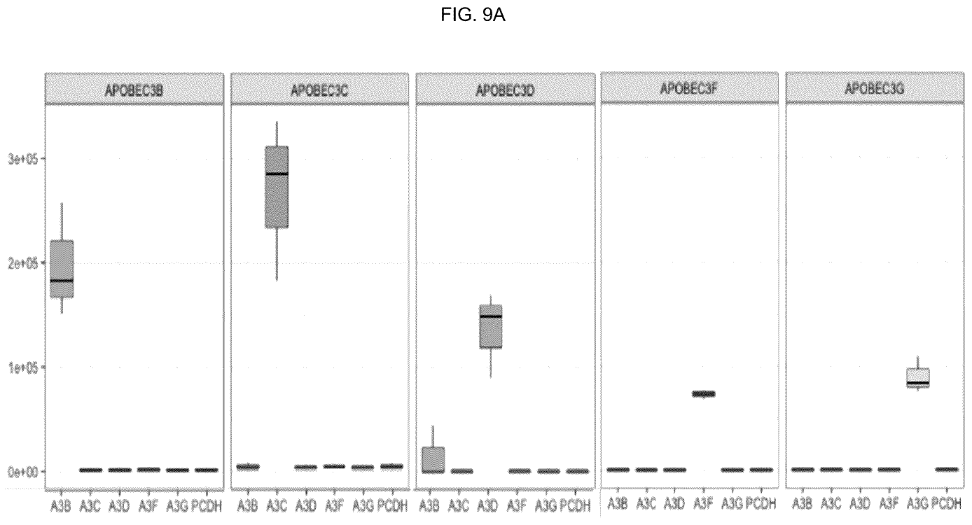

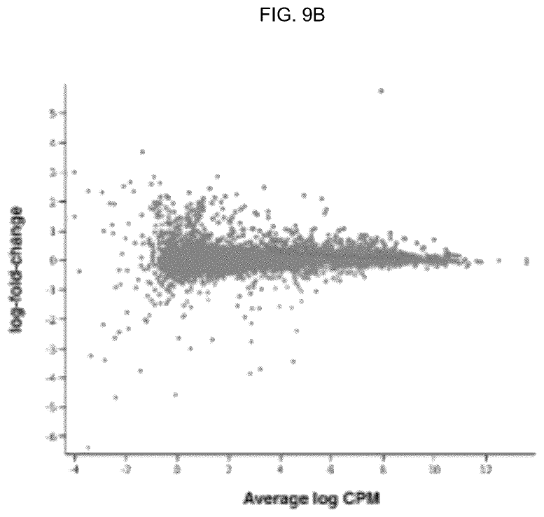

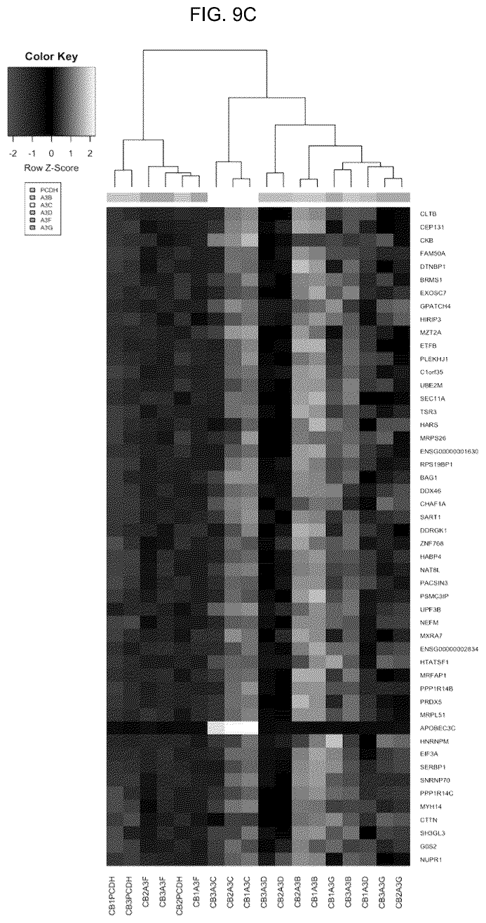

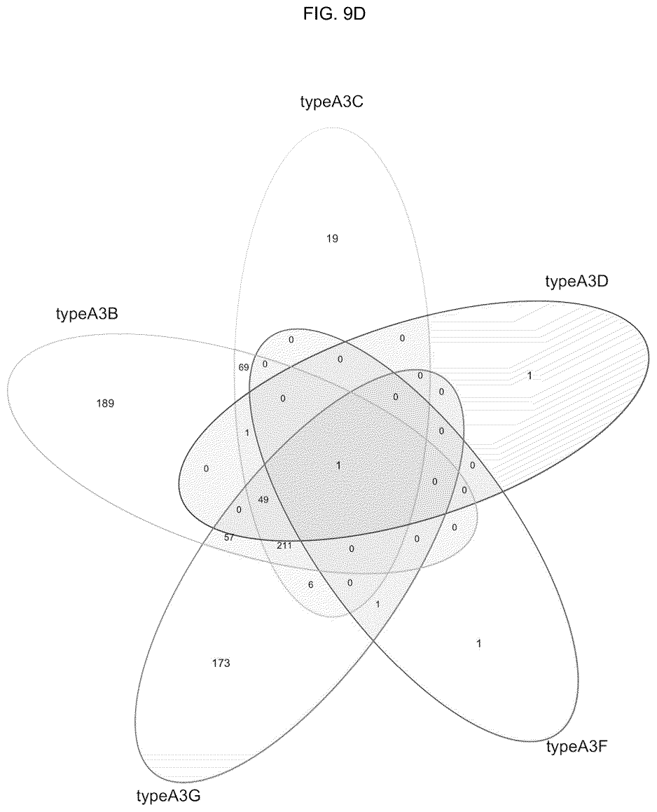

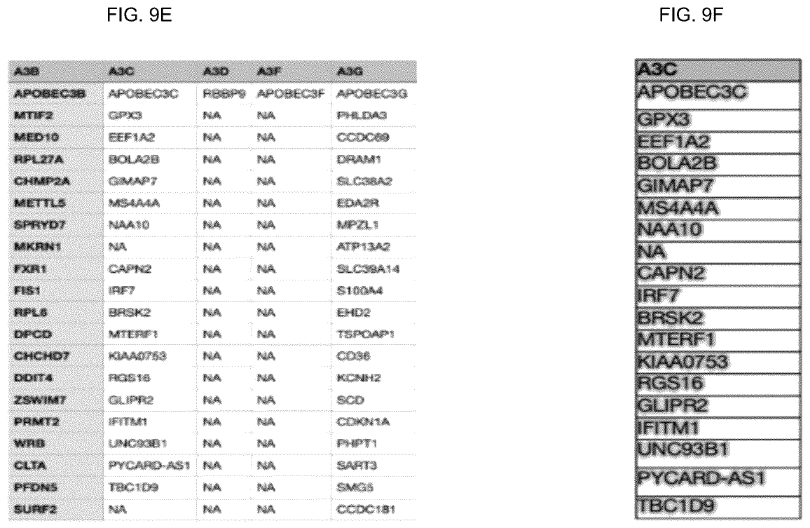

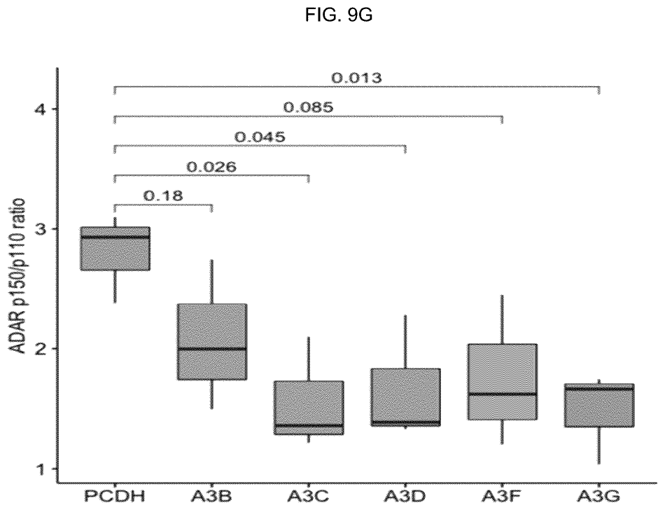

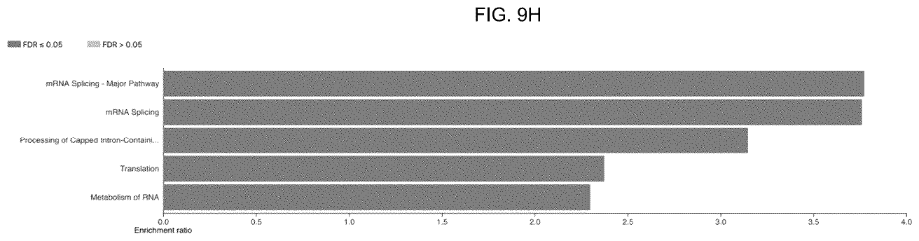

FIG. 9A-H: APOBEC3 overexpression and differential gene expression changesk: A. Relative APOBEC3C overexpression in CD34+ cord blood. B. MA plot of APOBEC3C overexpression. C. Heatmap showing top 50 differentially expressed genes after APOBEC3C overexpression. D. Venn diagram of APOBEC3 differentially expressed genes. E. Unique differentially expressed genes for each APOBEC3 family member. F. Unique differential expression and fold change of genes after APOBEC3C overexpression. Red represents significantly upregulated genes; blue represents significantly downregulated genes. G. ADAR1 p150/p110 expression after APOBEC3 overexpression compared to pCDH backbone control. H. Significantly enriched pathways found with GSEA Reactome analysis after APOBEC3C overexpression. Five major pathways were significantly enriched after A3C overexpression.

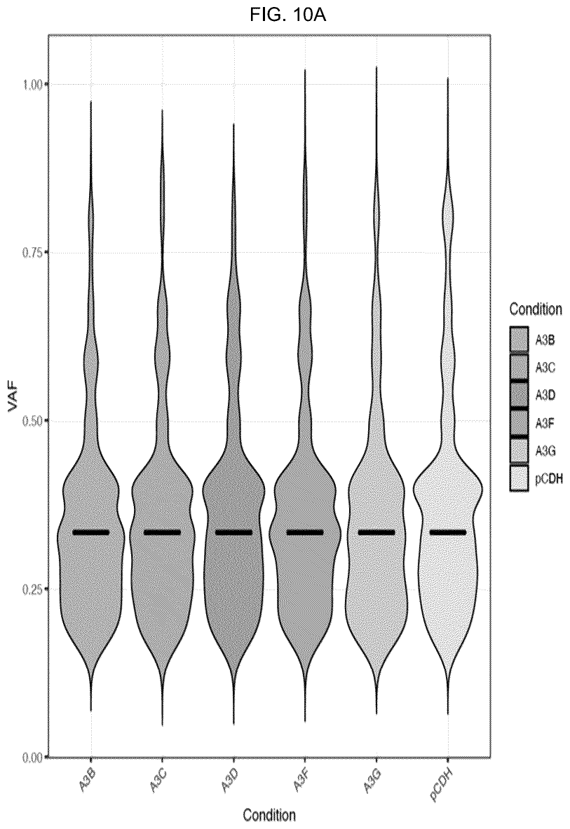

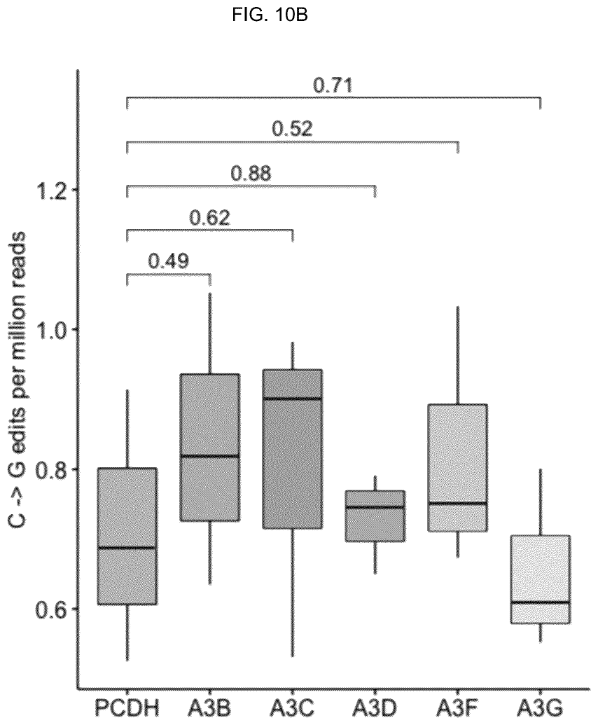

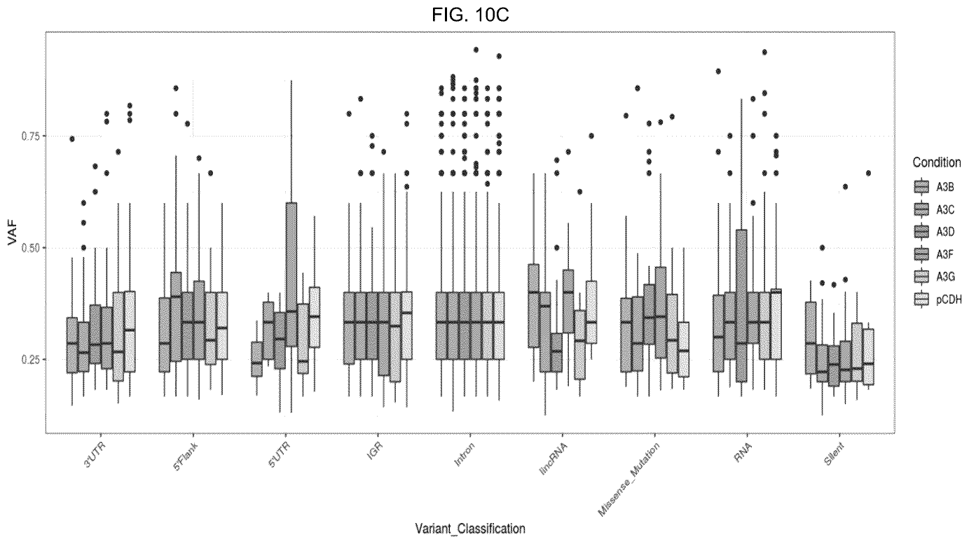

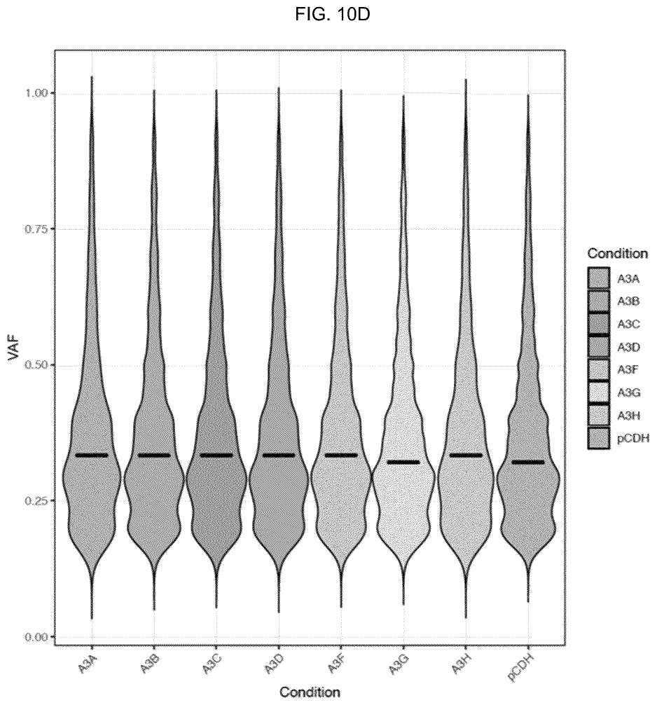

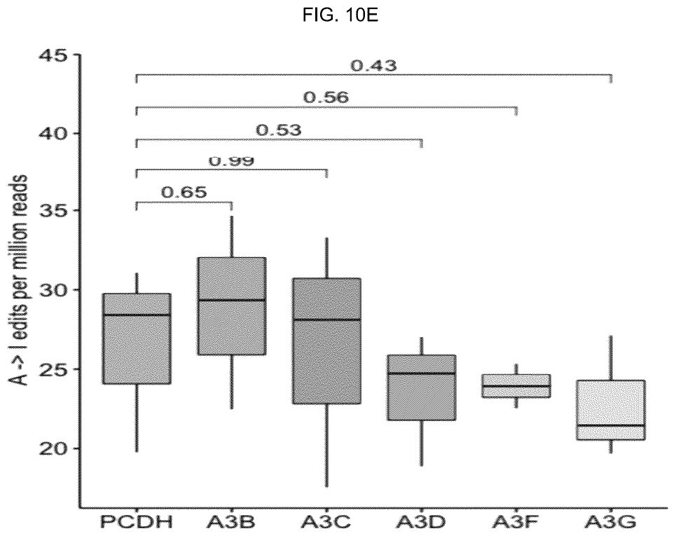

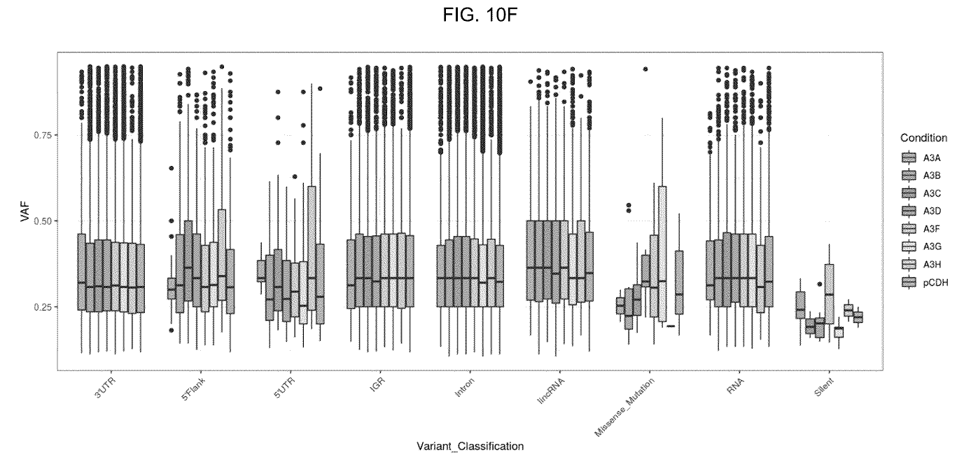

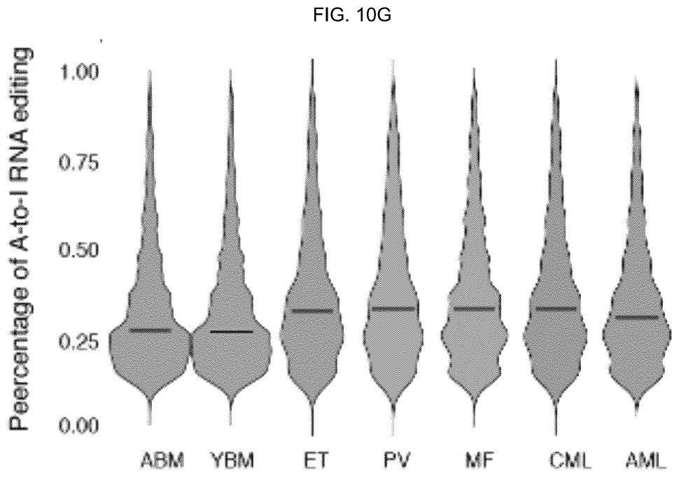

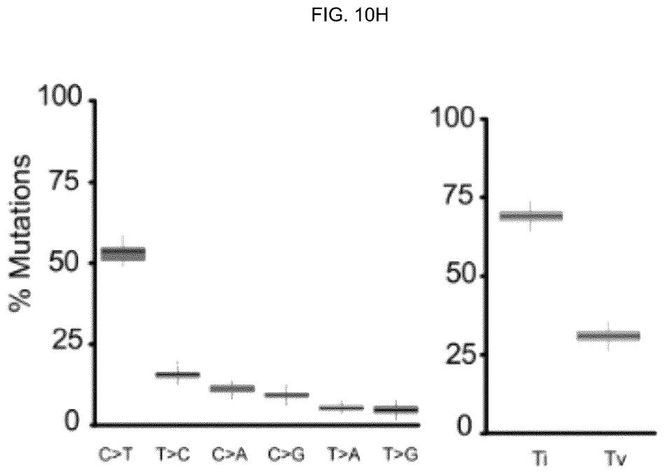

FIG. 10A-I: Editing profiles of normal and malignant hematopoietic cells: A.-C. C-to-U RNA editing in APOBEC3C overexpressed normal CD34+ cord blood cells. A. Variant allele frequency. B. Edited genes per sample. C. Editing stratified by variant classification. D.-F. A-to-I RNA editing in APOBEC3C overexpressed normal CD34+ cord blood cells. D. Variant allele frequency. E. A-to-I edits per million reads. F. Editing stratified by variant classification. G. Overall A-to-I RNA editing frequency by MPN stage H. A boxplot depicting the number of somatic DNA mutations in peripheral blood or saliva based on transitions (Tis) or transversions (Tvs) from myeloproliferative neoplasm patient samples. Both somatic and germline variants were included. I. A boxplot depicting the number of somatic DNA mutations in peripheral blood or saliva based on transitions (Tis) or transversions (Tvs). Both somatic and germline variants were included.

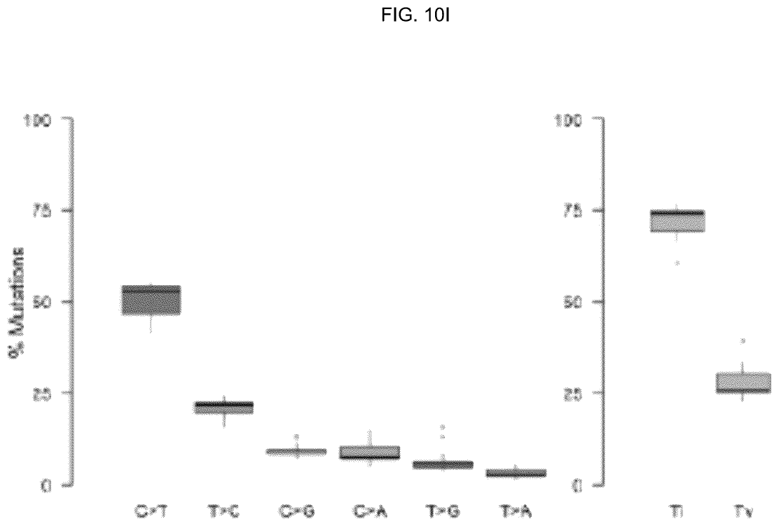

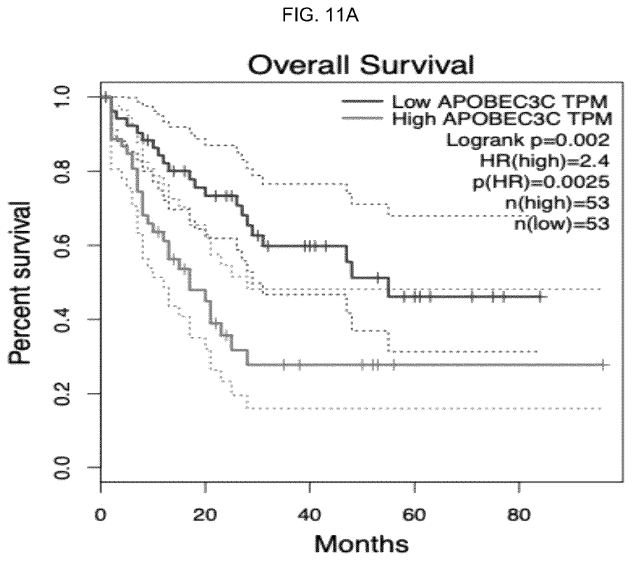

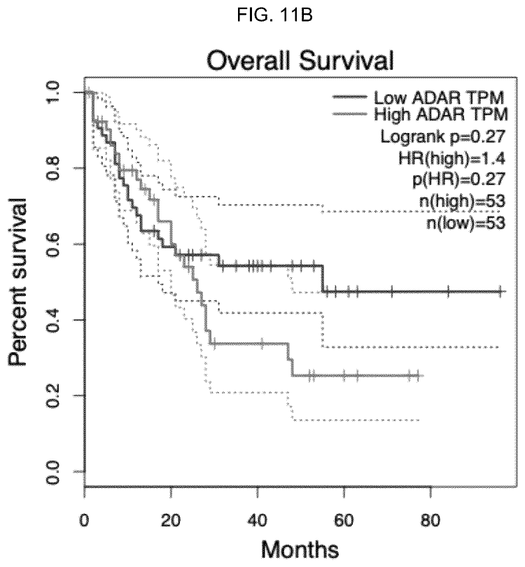

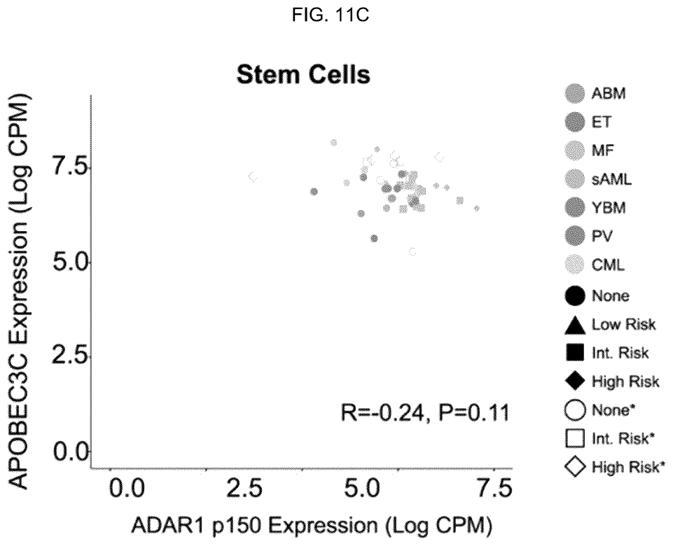

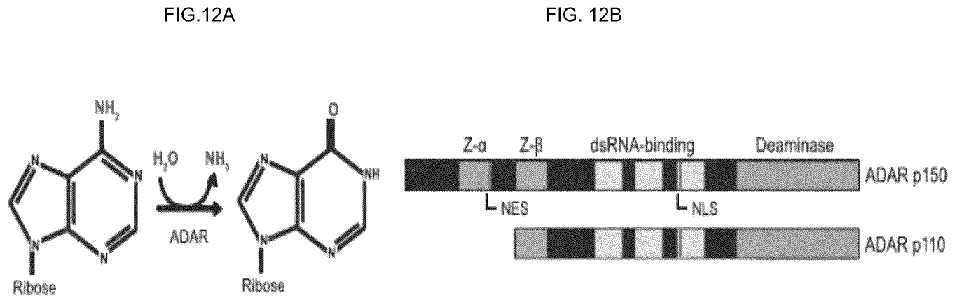

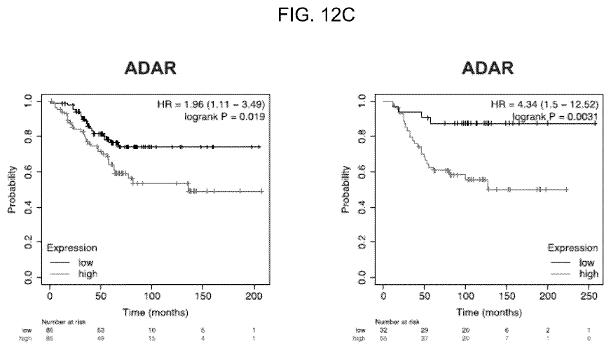

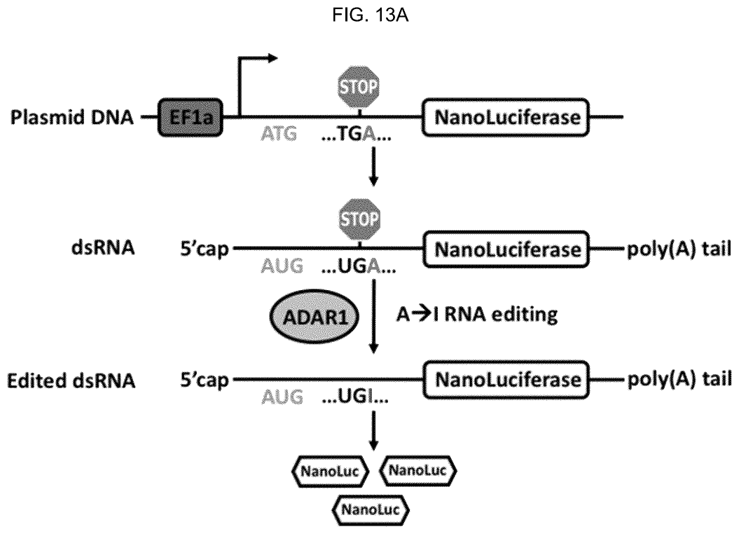

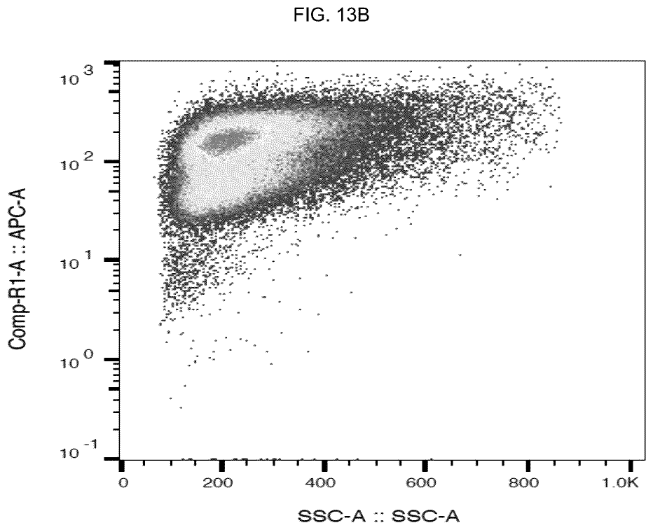

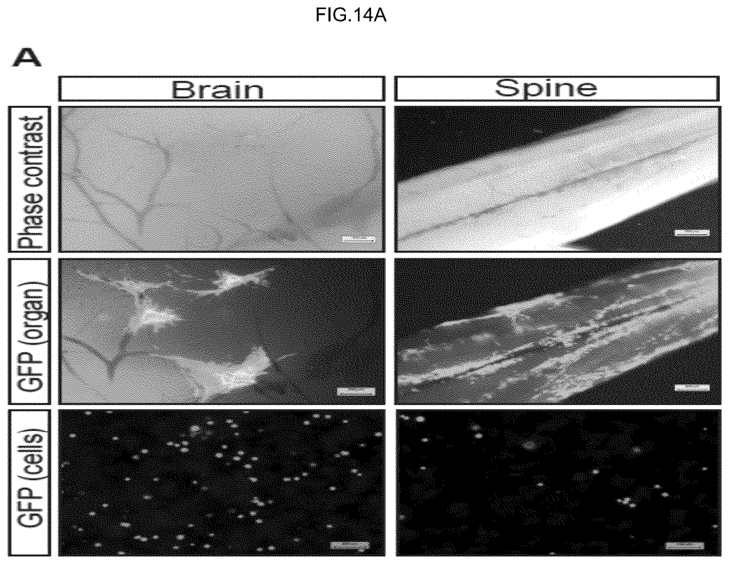

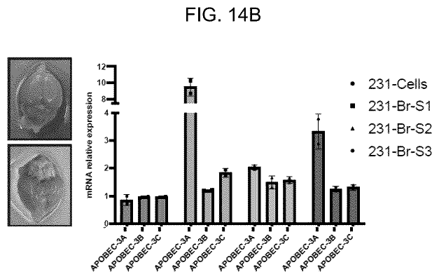

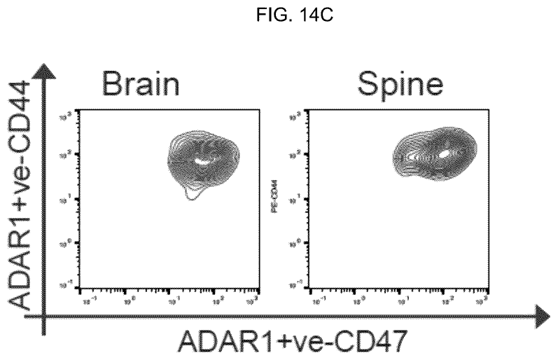

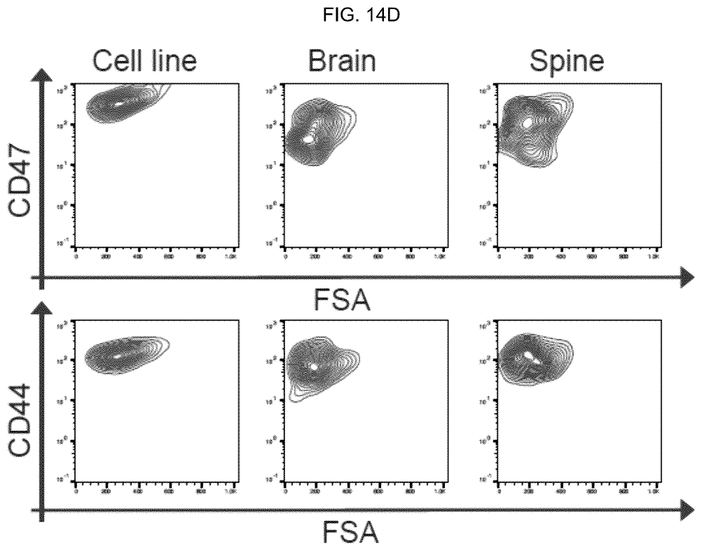

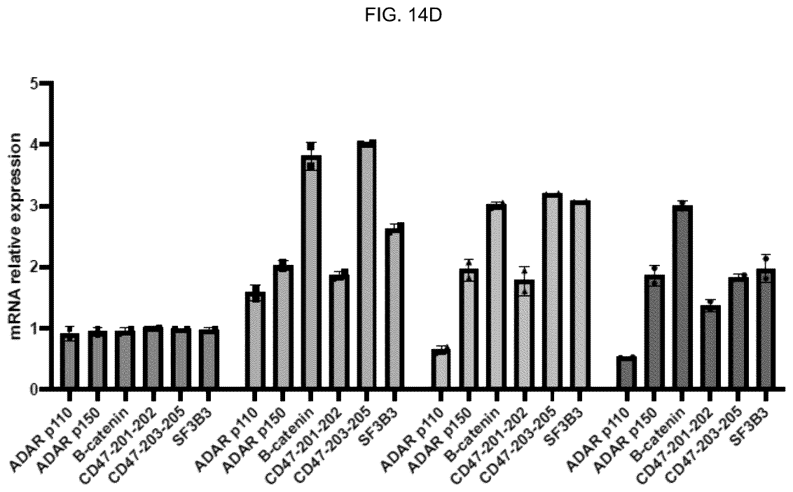

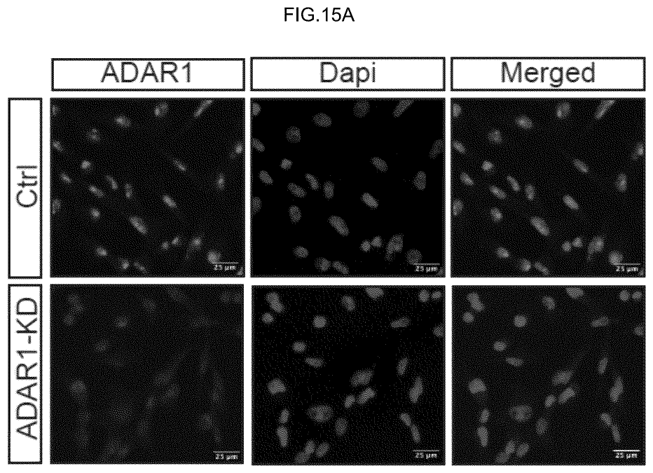

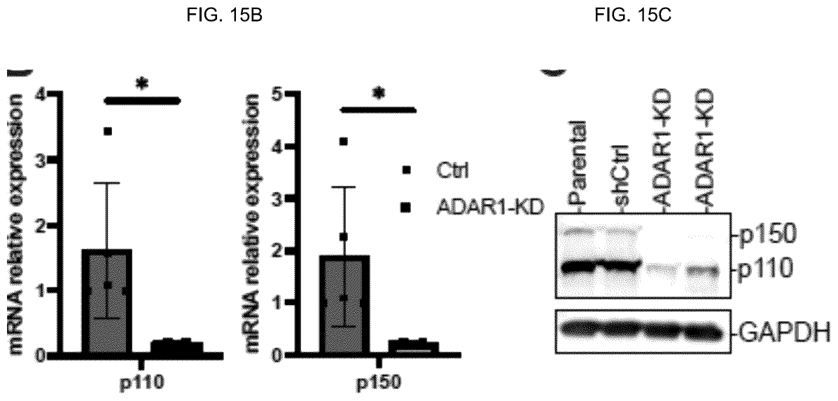

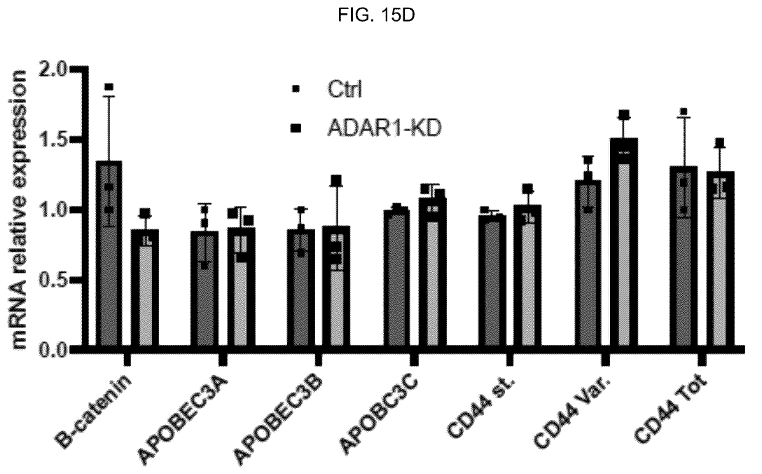

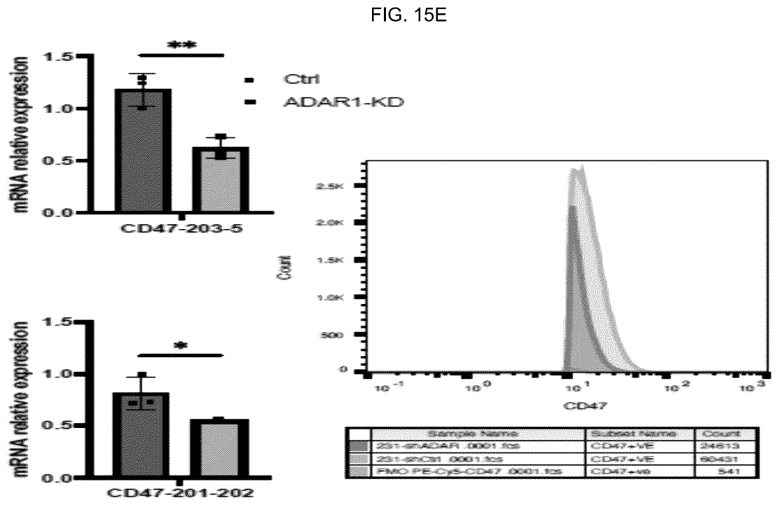

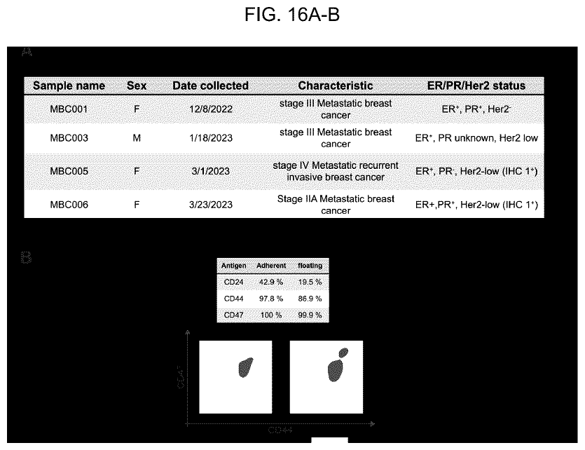

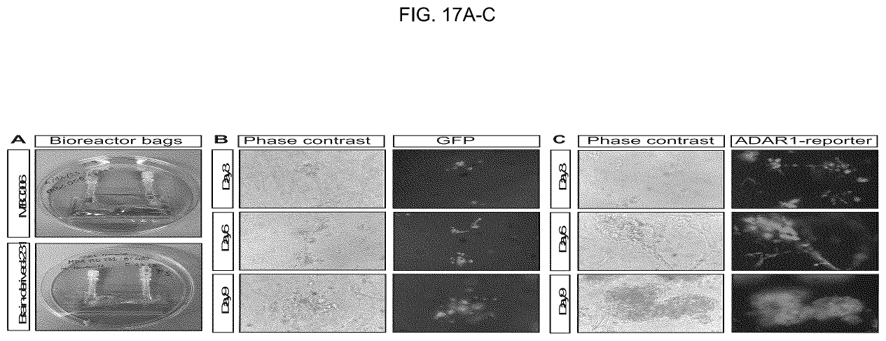

FIG. 11A-C: Prognostic implications: Prognostic implications for enhanced leukemic transformation and poor prognosis after deregulation of APOBEC3C and ADAR1, both innate immune deaminases. A. Kaplan-Meier plot showing overall survival of AML patients with high expression of APOBEC3C (red) or low expression of APOBEC3C (blue). B. Kaplan-Meier plot showing overall survival of AML patients with high or low ADAR1 expression (red) or low ADAR1 expression (blue). C. Correlation of APOBEC3C with ADAR1 p150 isoform in stem cells. Points are colored by phenotype.

### Example 3: Developing Organoids Models from Breast Cancer Metastasis

This example demonstrates exemplary methods for treating and diagnosing cancer.

Approximately 10 to 20% of patient diagnosed with breast cancer (BC) will develop metastatic disease which remains the leading cause of death. Breast is the second organ, after the lung, at high risk of developing brain metastases (BM). The incidence of BM is increasing due to improved treatments and detection of metastatic sites. Adenosine deaminase acting on dsRNA (ADAR1) is known to drive transcriptome remodeling through a plethora of mechanisms (including changes in amino acid sequence, mRNA splicing and/or canonical polyadenylation sites) which can also promote cancer stem cells progression. Our laboratory demonstrated that ADAR1 p150, the interferon-inducible isoform, favors the alternative splicing of proteins with invasive potential in leukemia stem cells. Moreover, in telomerase positive cancer, ADAR1 was found to promote telomerase activity by resolving R-loop formation in RNA:DNA hybrids of aberrant telomeric repeats. In BC, ADAR1 plays an essential role in cell survival due to increased apoptosis and reduced proliferation found in multiple BC cell lines engineered to express reduced levels of ADAR1. High levels of ADAR1 have been associated with shorter survival and disease progression in BC patients, however, the role of ADAR1 is only partially understood in the context of metastasis.

We aim to establish orthotopic and organoid models to better characterize ADAR1 activity in BC metastatic niches, with a special focus on the less studied BM. We are now in the process of collecting tumor biopsies from metastatic BC patients at the University of California, San Diego. Overall, this study will provide new insights into the mechanistic role of ADAR1 in breast metastatic disease and establish novel models for the development of new therapeutics.

### Background

Transcriptome remodeling led by RNA modifiers, such as the adenosine deaminase acting on dsRNA (ADAR1), is emerging as a tumor progression mechanism in cancer.

We hypothesized that ADAR1 may play a role in promoting breast cancer (BC) metastases, given the worse prognosis observed in high-grade tumor patients, especially in the HER2 positive subtype, which together with the triple negative (TNBC), are at high risk of developing brain metastases. Our laboratory demonstrated that inflammation driven ADAR1 p150 favors the alternative splicing of proteins with invasive potential in leukemia stem cells. Therefore, we sought to investigate ADAR1 p150 in BC metastases, with a special focus on the brain.

FIG. 1: High Levels of ADAR1 Correlate with Shorter OS in High-Grade BC.

(A) ADAR1 deaminase activity converts adenosines to inosines which are interpreted as guanosines, de facto changing the mRNA; (B) ADAR1 two main isoforms p110 and interferon-inducible p150 which expresses the Z-α binding domain for interacting with RNA/DNA in a conformation; (C) reduced Overall Survival (OS) from mRNA dataset of grade three, HER2 positive BC tumors which underwent endocrine therapies (p=0.019), (left) and reduced OS from protein dataset in grade three BC compared with control (p=0031), (right). KM plotter dataset.

### Methodology

We developed a mouse model based on intra-cerebroventricular (ICV) injections of cancer cells transduced with an ADAR1-GFP reporter into the brain of Rag2−/− γc−/− mice at postnatal day 2.

### FIG. 2: Workflow to Establish ADAR1-Driven Brain Metastases Mouse Model.

(A) Cancer cells are transduced with a nanoluc-luciferase reporter construct for tracing ADAR1 activity; (B) neonatal pups are implanted through ICV with transduced cells; (C) tumor growth (3-6 weeks for the MDA-MD-231 cells); (D) FACS-sorting of GFP+-tumor cells; (E) cells collection for downstream analyses.

### Orthotropic Model of Breast Cancer Brain Metastases

ADAR1-GFP metastases from ICV implants of the MDA-MB-231 TNBC line were visible at three weeks post implants in the brain and spinal cord of Rag2−/− γc−/− mice. FACS-analysis of brain metastases revealed upregulation of ADAR1 p150 together with WNT/β-catenin pathway and APOBEC mutagenesis genes. Changes in splicing and regulation of cancer stem cells genes was also observed. Especially for CD47, the “do noteat me” signal that fuels cancer stem cells progression.

### FIG. 3: ADAR1 Potentiates Cancer Stem Cells Aggressiveness.

(A) Pictures of MDA-MD-231 ADAR1-reporter metastases from organs (top and middle panel) and digested tissue (bottom panel), (B) picture of a MDA-MD-231 implanted brain at three weeks post-ICV, (C) FACS analysis of one brain and one spine from total brain extract co-expressing ADAR1-GFP positive cells, CD44 and CD47; (D) FACS contour plot of CD44 and CD47 displaying different populations among the parental cell line versus brain and spine metastases, (E) RT-PCR from GFP-FACS-sorted brain metastases (231-Br S1,S2 and S3) and non-transplanted cell line (231-Cells).

### Exploring ADAR1 Functions In Vitro

To mechanistically investigate the role of ADAR1 in BC context we engineered the MDA-MB-231 TNBC line to express lower levels of ADAR1. We were able to confirm down-regulation of CD47 specific isoforms in vitro upon ADAR1 knock-down (KD).

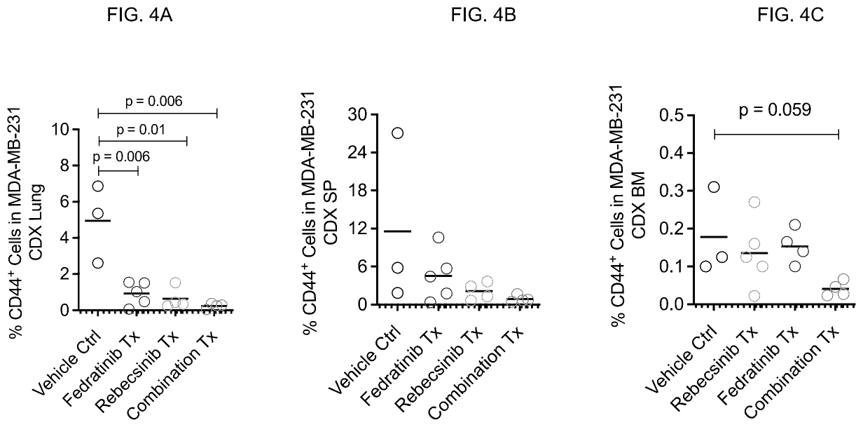

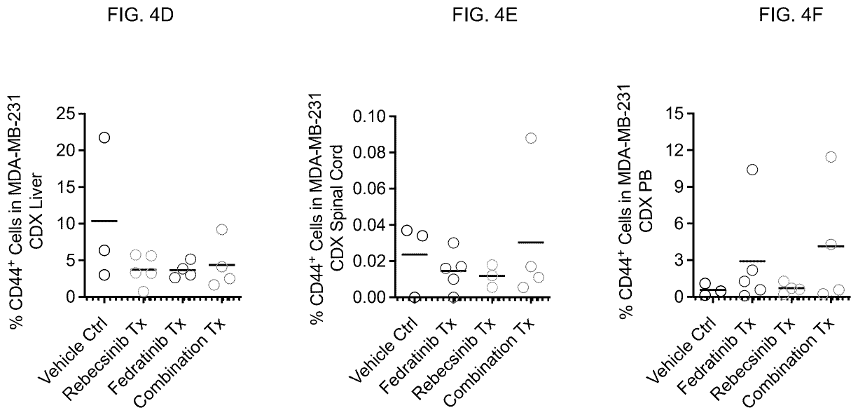

### FIG. 4: ADAR1 Knock-Down Reduces CD47 Expression.

(A) IF of ADAR1 in the MDA-MD-231 cells transduced with sh-scramble (Ctrl) or sh-ADAR1 (ADAR1-KD). Decreased ADAR1 intensity is visible in the ADAR1-KD; (B) RT-PCR confirming down-regulation of ADAR1 p110 and p 150 isoforms, (Two-tailed t-test p value=0.0348, for p110 and 0.0467 for p150); (C) WB showing decreased protein levels in two MDA-MD-231 clones compared with the parental and transduced line; (D) RT-PCR from three independent replicates of multiple genes involved in the regulation of BC metastases; (E) CD47 KD confirmed via RT-PCR in Ctrl and ADAR1-KD (Two-tailed t-test, p value=0.0448 for CD47 201-202, p value=0.0066 CD47 203-5) (left), CD47-FACS histogram (right).

### Samples Collection and Banking

We established a collaboration with the clinicians at UCSD to generate primary metastatic models. We are now collecting and profiling metastases from multiple sites.

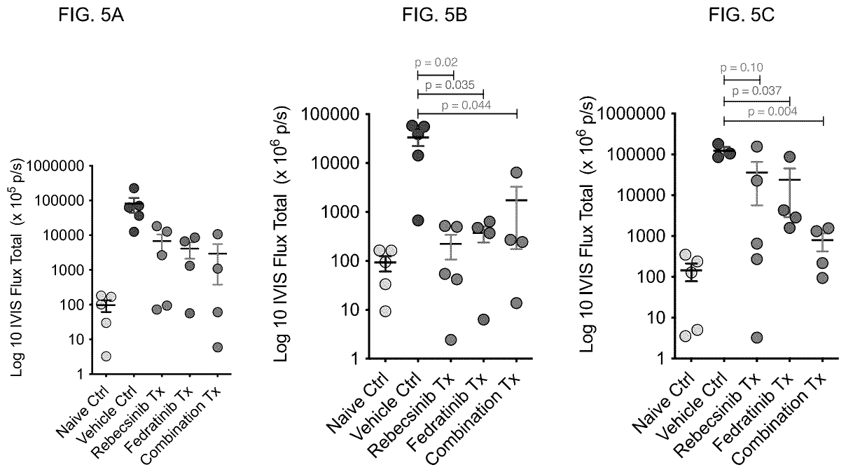

FIG. 5: Characterizing Samples from Breast Cancer Metastatic Sites.

(A) Metastatic apheresis (MBC003) and malignant pleural effusions (mPE) (others) patient specification table, (B) example of breast cancer stem cells profiling for the MBC006.

### Modeling Metastases In Vitro

Pioneering experiments from the Jamieson laboratory taking place in low-earth orbit have shown the long-term stability of cultures in bioreactors.

### FIG. 6: Tumoroids Grown in a Bioreactor Bag, an Augmented In Vitro Model.

(A) Example of bioreactor bags for BC primary tumoroids from the mPE MB6006 with pCDH-GFP (top) and Br-231 cells (bottom), (B) MBC006 cells grown in BC conditional media, objective 20×, (C) FACS-sorted Br-231 cells with the ADAR1 reporter grown in neural conditional media, objective 20×.

### Conclusions

This study introduces an ICV-based model for tracing ADAR1 activity in BC brain metastases. Our data suggest that ADAR1 p150 driven metastases rely on the wnt/β-catenin pathway and differential splicing of key genes, such as CD47, to promote cell proliferation.
We propose that ADAR1 collaborates with the metastatic niche to fuel cell proliferation, since the phenotype observed is specific to the brain microenvironment.
Both ICV-mouse metastatic model and mPE-derived tumoroids represent a robust platform for uncovering ADAR-1 functions in the BC metastatic environment.

### Example 4: CD44+ Expression in CDX (Cell Line Derived Xenograft) MDA-MB-231 Xenograft Mouse Model

Ten thousand (10k) MDA-MB-231 cells were intrahepatically transplanted into Rag2 neonatal mice, then treated with fedratinib, rebecsinib, or a combination of fedratinib and rebecsinib. CD44+ cells were measured in various tissues (lung, spleen (SP), bone marrow (BM), liver, spinal cord, and peripheral blood (PB)) of the mice and plotted in FIG. 4, which shows that the combination of fedratinib and rebecsinib significantly inhibited CD44+ breast cancer cells.

### Example 5

Ten thousand (10k) MDA-MB-231 cells were intrahepatically transplanted into Rag2 neonatal mice, then treated with fedratinib, rebecsinib, or a combination of fedratinib and rebecsinib. IVIS images were taken at 6 weeks after transplant (FIG. 5A), after two weeks of therapy (FIG. 5B), and one week after therapy was completed (FIG. 5C). The IVIS signals from the mice were graphed by groups.

The IVIS images of these mice were taken at 0 day after completing the dosing plan and are graphed by their total flux. The results show that the total flux in the rebecsinib, fedratinib, and combination treated mice was significantly decreased compared to vehicle control.

### Example 6: Rebecsinib Treatment Spares Human CD45+ADAR1−GFP+ Cells and CD45+CD3+ADAR1−GFP+ Cells in aNBM SAK148 PDX Mouse Peripheral Blood (PB), Spleen (SP), and Bone Marrow (BM)

Aged normal bone marrow (aNBM) PDX mouse models SAK148, which were established with 80-100K ADAR1-reporter transduced CD34+ cells by IV injection into NSG-SGM3 mice at the age of 5-6 weeks. When the models were ready, the mice were treated with either vehicle or rebecsinib (10 mg/kg, IV, BIW×2). The mice were sacrificed upon completion of the dosing plan, and single cells from PB, SP, and BM were collected and stained with mAbs for CD45, CD3, CD14, and CD19. The results demonstrated that rebecsinib treatment spares the cell populations of both CD45+GFP+ cells and CD45+CD3+GFP+ cells in PB, SP, and BM.

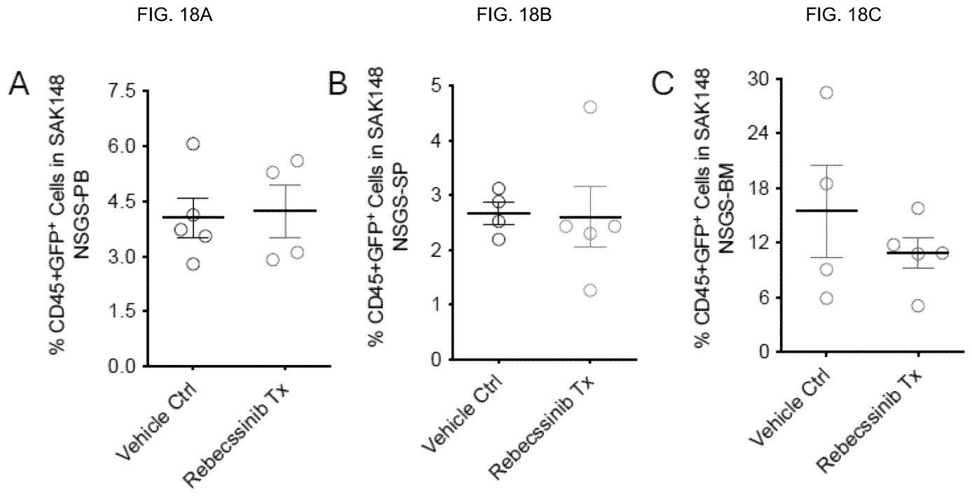

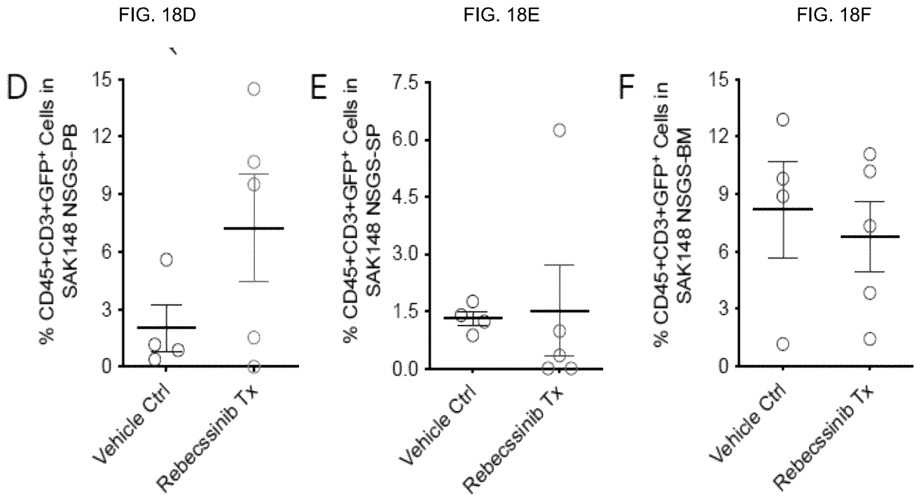

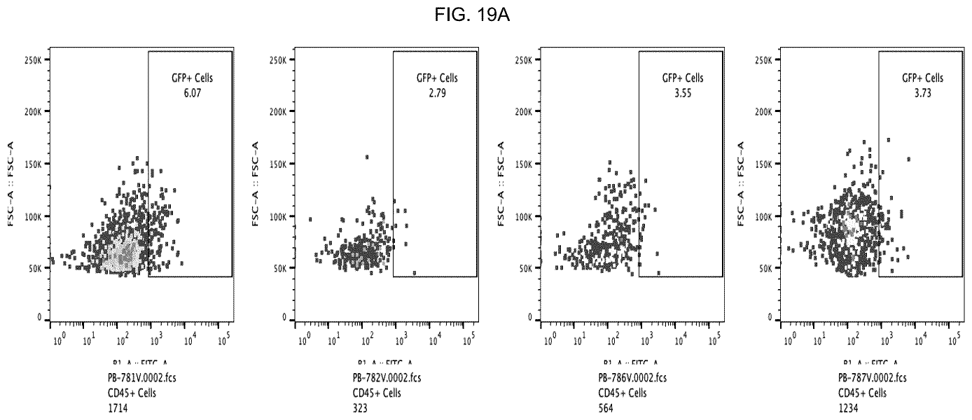

FIG. 18A-F graphically illustrate data demonstrating that rebecsinib treatment spares human CD45+ADAR1−GFP+ cells and CD45+CD3+ADAR1−GFP+ cells in aNBM SAK148 PDX mouse peripheral blood (PB), spleen (SP), and bone marrow (BM):

FIG. 18A graphically illustrates levels of CD45+GFP+ cells in PB after rebecsinib treatment;

FIG. 18B graphically illustrates levels of CD45+GFP+ cells in SP after rebecsinib treatment;

FIG. 18C graphically illustrates levels of CD45+GFP+ cells in BM after rebecsinib treatment;

FIG. 18D graphically illustrates levels of CD45+CD3+GFP+ cells in PB after rebecsinib treatment;

FIG. 18E graphically illustrates levels of CD45+CD3+GFP+ cells in SP after rebecsinib treatment; and

FIG. 18F graphically illustrates levels of CD45+CD3+GFP+ cells in BM after rebecsinib treatment.

### Example 7: Rebecsinib Tx Spares Human CD45+ADAR1−GFP+ Cells and CD45+CD3+ADAR1−GFP+ Cells in aNBM SAK148 PDX Mouse PB

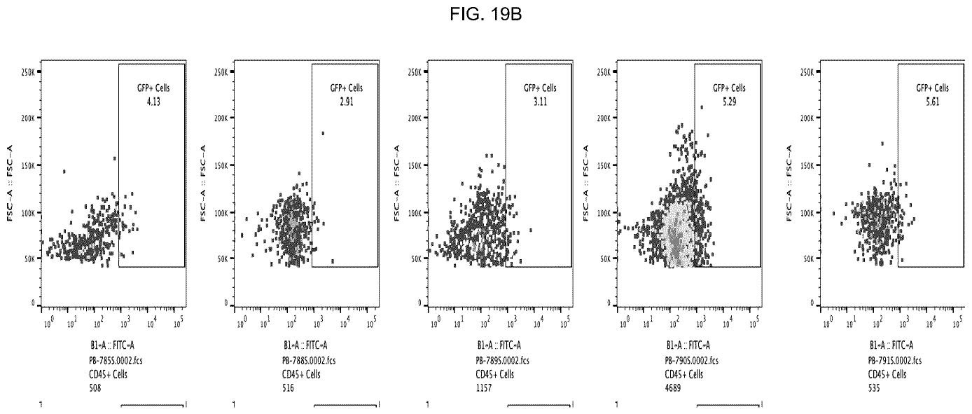

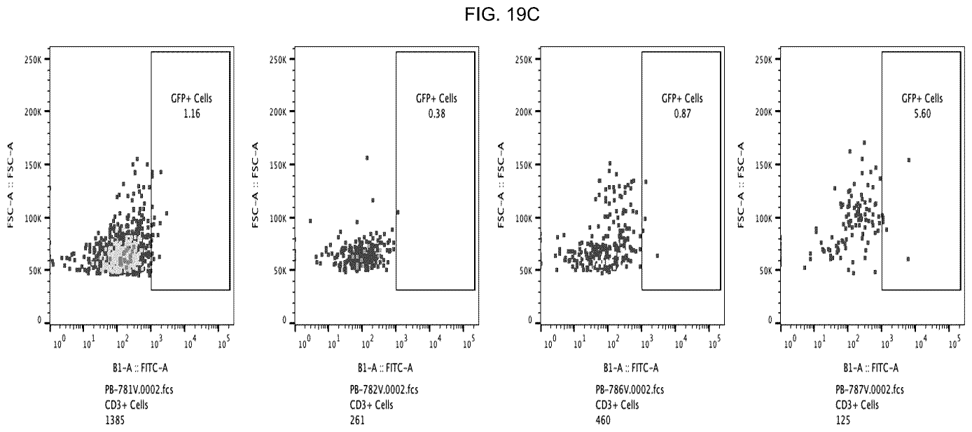

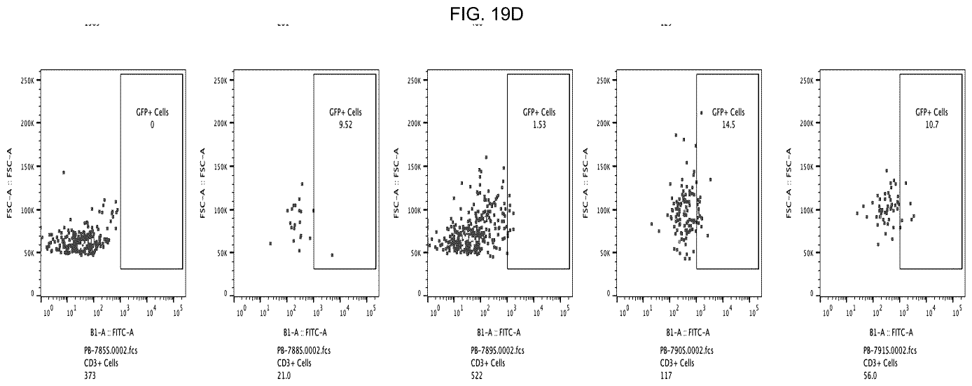

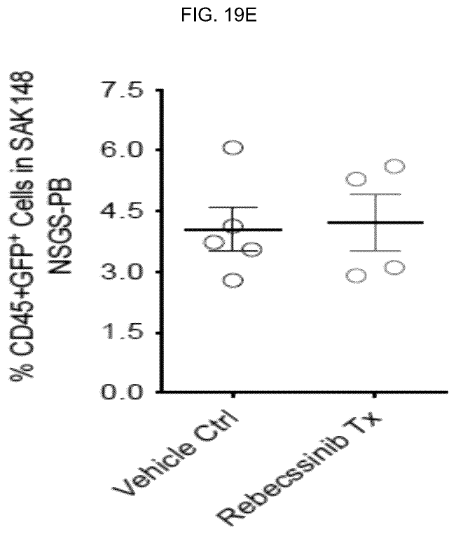

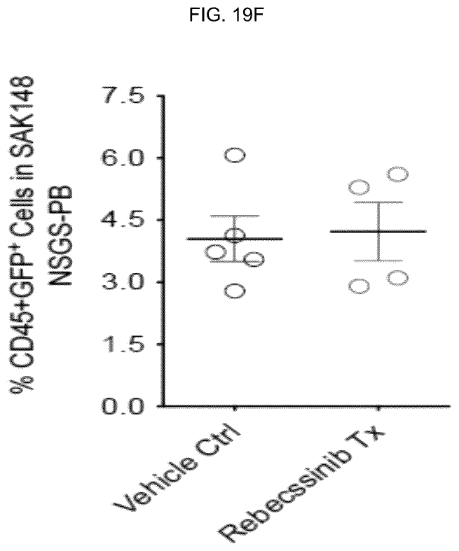

FIG. 19A-F graphically illustrate data demonstrating that rebecsinib treatment (tx) spares human CD45+ADAR1−GFP+ Cells and CD45+CD3+ADAR1−GFP+ Cells in aNBM SAK148 PDX Mouse PB.

FIG. 19A-B illustrate cell sorting (FACS) scans illustrating levels of CD45+GFP+ cells after rebecsinib treatment;

FIG. 19C-D illustrate cell sorting (FACS) scans illustrating levels of CD45+CD3+GFP+ cells after rebecsinib treatment;

FIG. 19E graphically illustrates data from the cell sorting (FACS) scans illustrated in FIG. 19A-B; and

FIG. 19F graphically illustrates data from the cell sorting (FACS) scans illustrated in FIG. 19C-D.

### Example 8: Rebecsinib Tx Spares Human CD45+ADAR1−GFP+ Cells and CD45+CD3+ADAR1−GFP+ Cells in aNBM SAK148 PDX Mouse SP

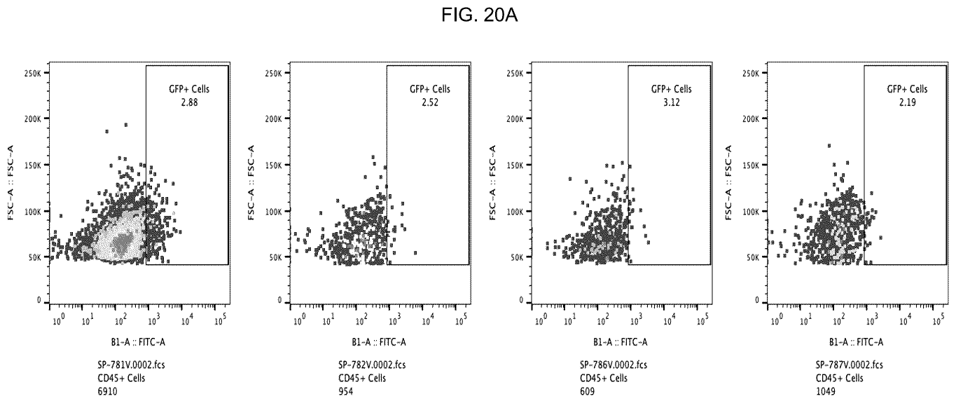

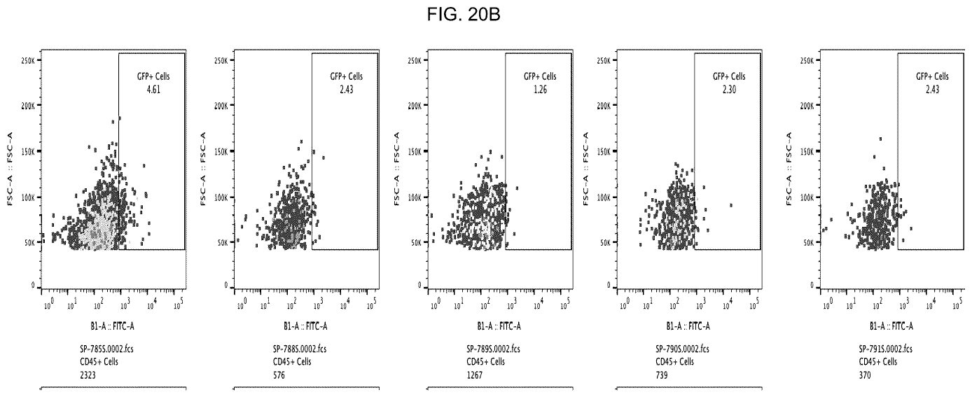

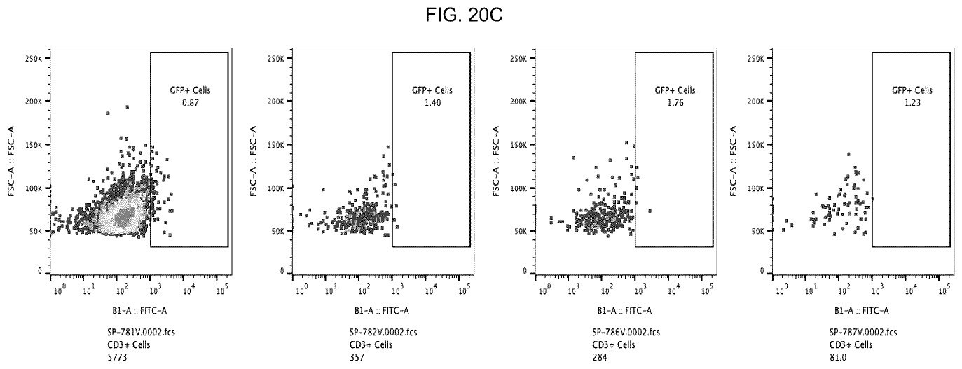

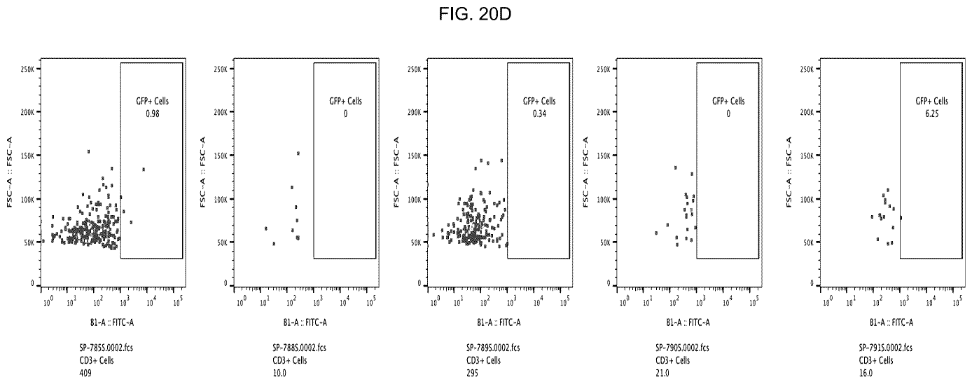

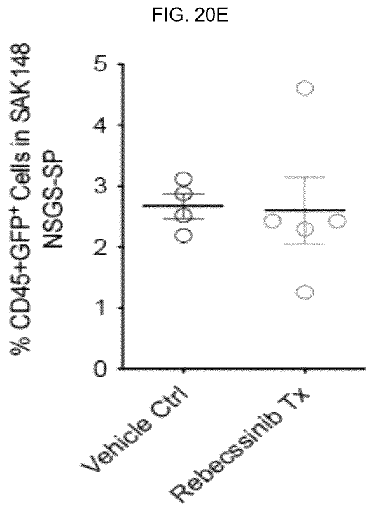

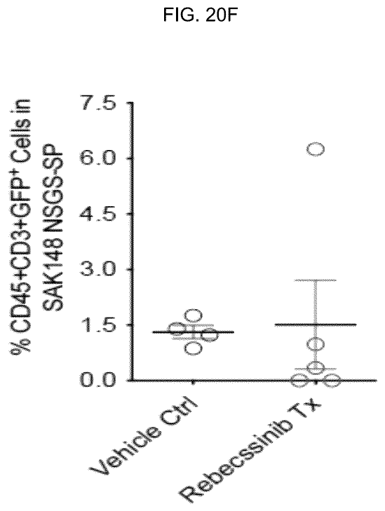

FIG. 20A-F graphically illustrate data demonstrating that rebecsinib treatment (tx) spares human CD45+ADAR1−GFP+ cells and CD45+CD3+ADAR1−GFP+ cells in aNBM SAK148 PDX mouse SP.

FIG. 20A-B illustrate cell sorting (FACS) scans illustrating levels of CD45+GFP+ cells after rebecsinib treatment;

FIG. 20C-D illustrate cell sorting (FACS) scans illustrating levels of CD45+CD3+GFP+ cells after rebecsinib treatment;

FIG. 20E graphically illustrates data from the cell sorting (FACS) scans illustrated in FIG. 20A-B; and

FIG. 20F graphically illustrates data from the cell sorting (FACS) scans illustrated in FIG. 20C-D.

### Example 9: Rebecsinib Tx Spares Human CD45+ADAR1−GFP+ Cells and CD45+CD3+ADAR1−GFP+ Cells in aNBM SAK148 PDX Mouse BM

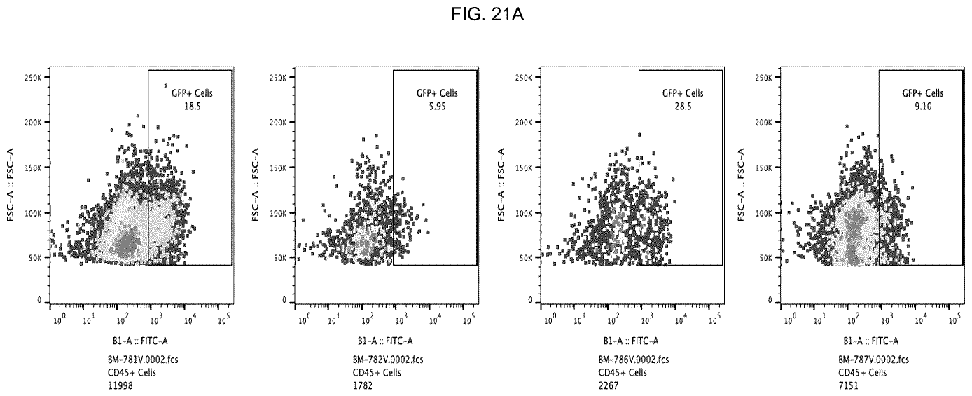

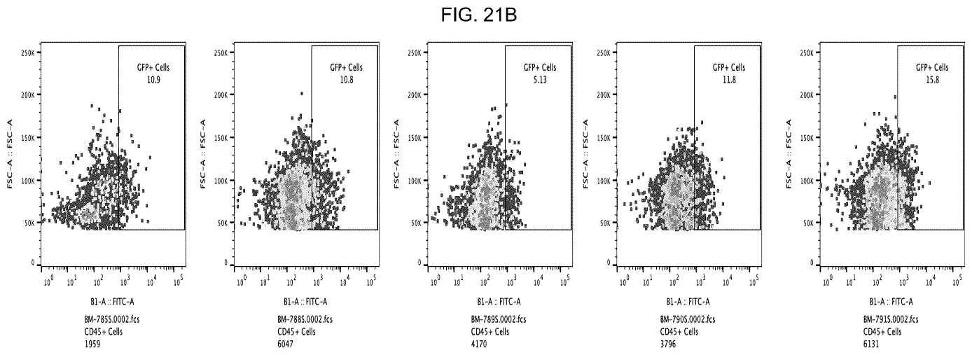

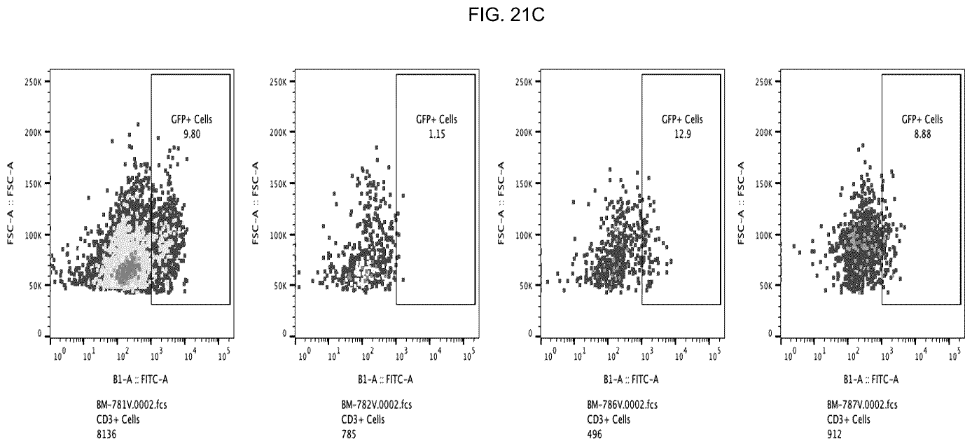

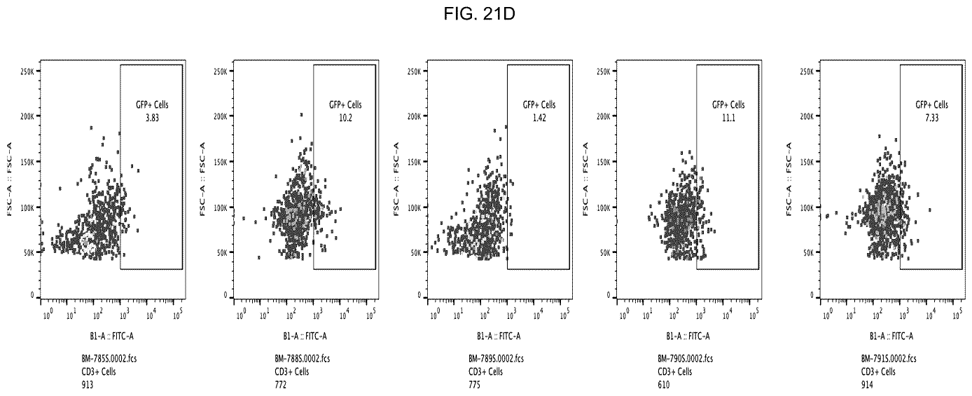

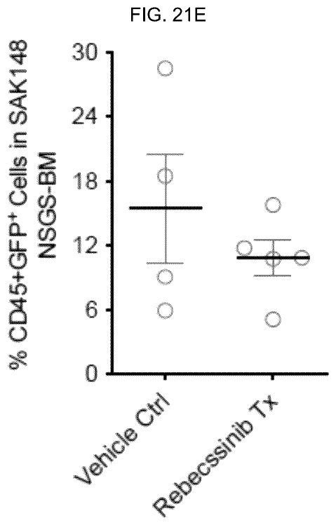

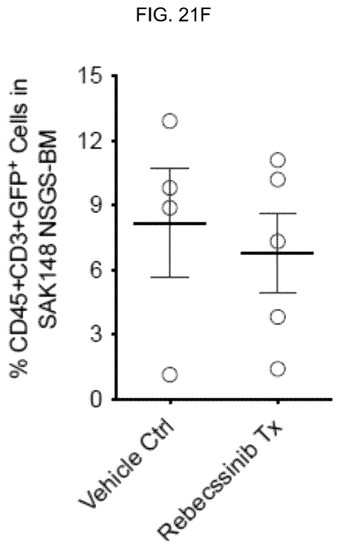

FIG. 21A-F graphically illustrate data demonstrating that rebecsinib treatment (tx) spares human CD45+ADAR1−GFP+ cells and CD45+CD3+ADAR1−GFP+ cells in aNBM SAK148 PDX mouse BM.

FIG. 21A-B illustrate cell sorting (FACS) scans illustrating levels of CD45+GFP+ cells after rebecsinib treatment;

FIG. 21C-D illustrate cell sorting (FACS) scans illustrating levels of CD45+CD3+GFP+ cells after rebecsinib treatment;

FIG. 21E graphically illustrates data from the cell sorting (FACS) scans illustrated in FIG. 21A-B; and

FIG. 21F graphically illustrates data from the cell sorting (FACS) scans illustrated in FIG. 21C-D.

### Example 10: Rebecsinib Tx Spares Human CD45+ADAR1−GFP+ Cells and CD45+CD3+ADAR1−GFP+ Cells in aNBM SAK378 PDX Mouse PB, SP, and BM

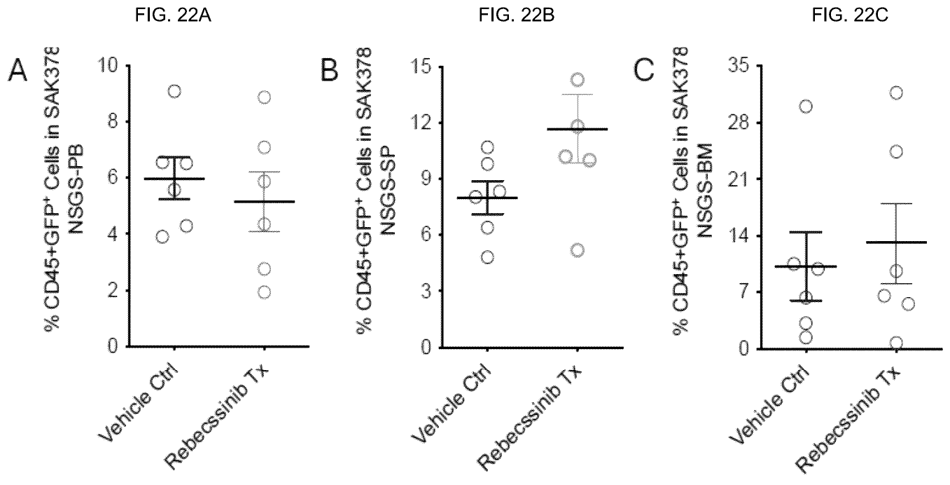

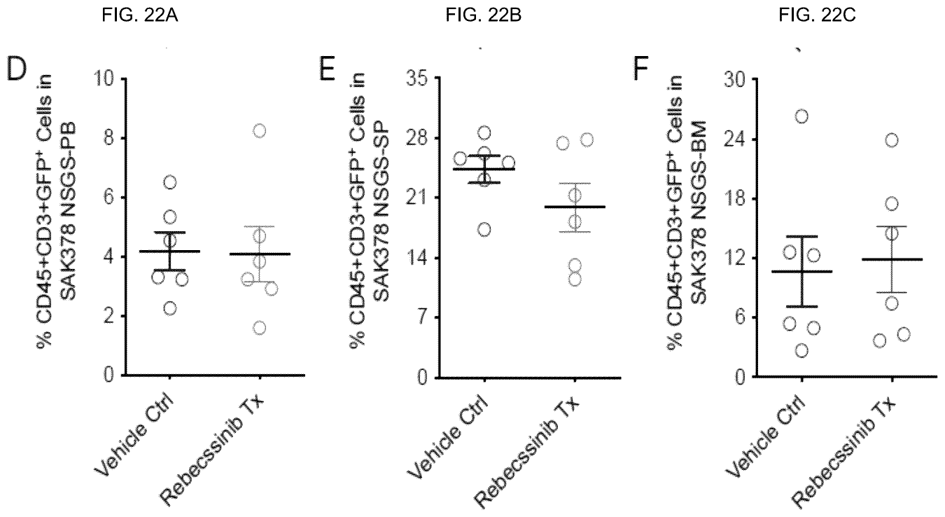

FIG. 22A-F illustrate data demonstrating that rebecsinib treatment (tx) spares human CD45+ADAR1−GFP+ cells and CD45+CD3+ADAR1−GFP+ cells in aNBM SAK378 PDX Mouse PB, SP, and BM.

FIG. 22A illustrates CD45+GFP+ cells in PB after either vehicle or rebecsinib treatment;

FIG. 22B illustrates CD45+GFP+ cells in SP after either vehicle or rebecsinib treatment;

FIG. 22C illustrates CD45+GFP+ cells in BM after either vehicle or rebecsinib treatment;

FIG. 22D illustrates CD45+CD3+GFP+ cells in PB after either vehicle or rebecsinib treatment;

FIG. 22E illustrates CD45+CD3+GFP+ cells in SP cells in PB after either vehicle or rebecsinib treatment; and

FIG. 22F illustrates CD45+CD3+GFP+ cells in BM cells in PB after either vehicle or rebecsinib treatment.

Aged normal bone marrow (aNBM) PDX mouse models SAK378 were established with 80-100K ADAR1-reporter transduced CD34+ cells by IV injection into NSG-SGM3 mice at the age of 5-6 weeks. When the models were ready, the mice were treated with either vehicle or rebecsinib (10 mg/kg, IV, BIW×2). The mice were sacrificed upon completion of the dosing plan, and single cells from PB, SP, and BM were collected and stained with mAbs for CD45, CD3, CD14, and CD19. The results demonstrated that rebecsinib treatment spares the cell populations of both CD45+GFP+ cells and CD45+CD3+GFP+ cells in PB, SP, and BM.

### Example 11: Rebecsinib Treatment (tx) Spares Human CD45+ADAR1−GFP+ Cells and CD45+CD3+ADAR1−GFP+ Cells in aNBM SAK378 PDX Mouse PB

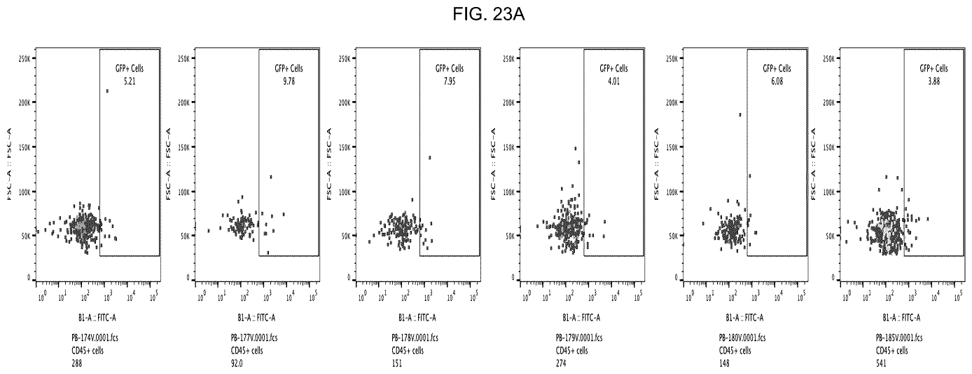

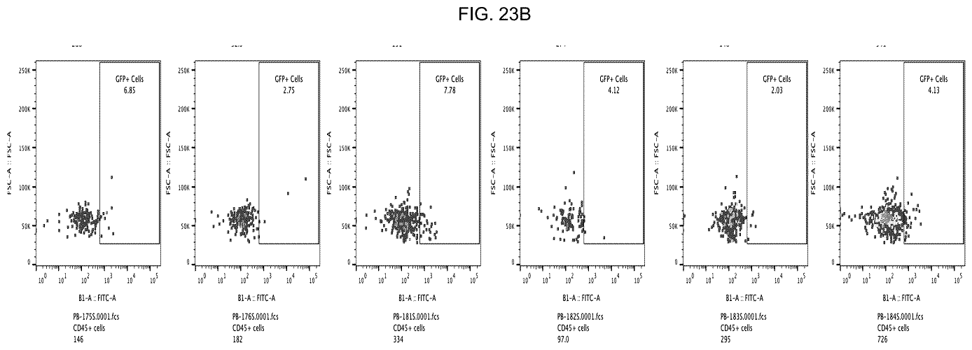

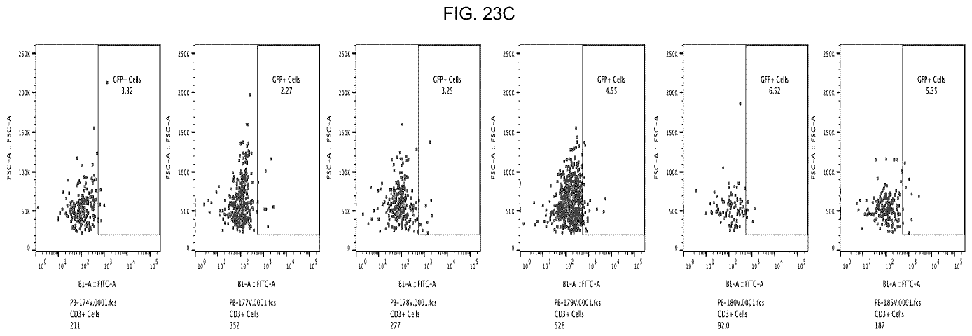

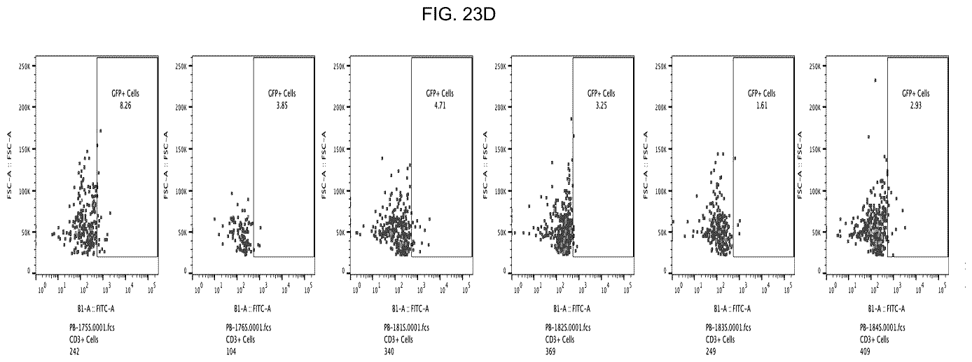

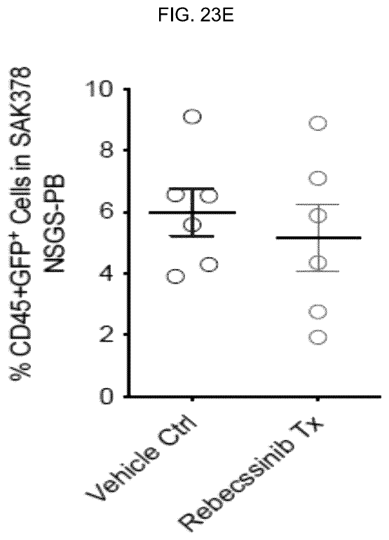

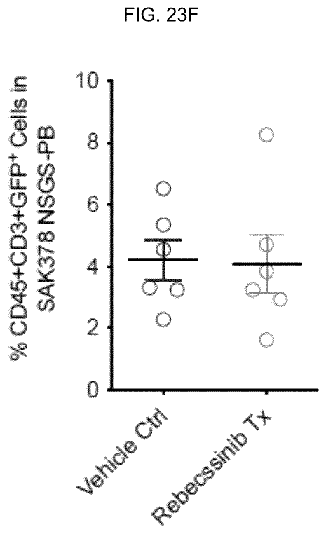

FIG. 23A-B illustrate cell sorting (FACS) scans illustrating levels of CD45+GFP+ cells after rebecsinib treatment;

FIG. 23C-D illustrate cell sorting (FACS) scans illustrating levels of CD45+CD3+GFP+ cells after rebecsinib treatment;

FIG. 23E graphically illustrates data from the cell sorting (FACS) scans illustrated in FIG. 23A-B; and

FIG. 23F graphically illustrates data from the cell sorting (FACS) scans illustrated in FIG. 23C-D.

### Example 12: Rebecsinib Treatment (tx) Spares Human CD45+ADAR1−GFP+ Cells and CD45+CD3+ADAR1−GFP+ Cells in aNBM SAK378 PDX Mouse SP

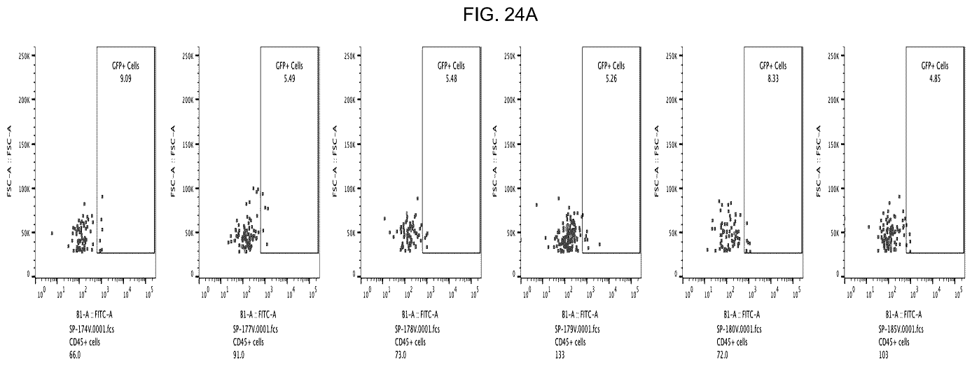

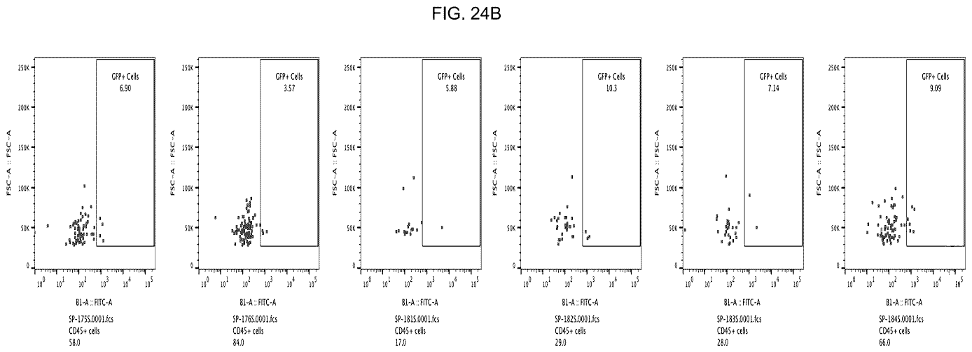

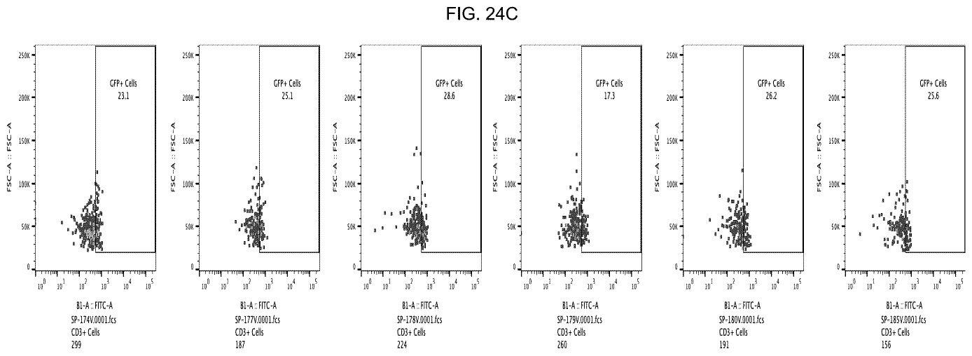

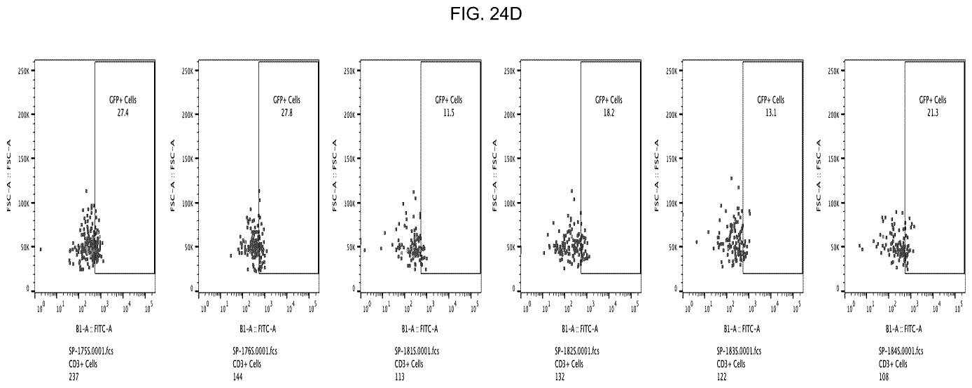

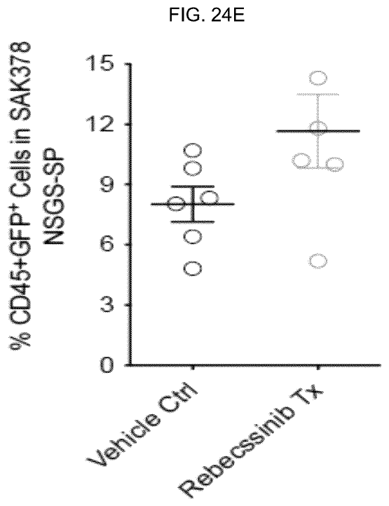

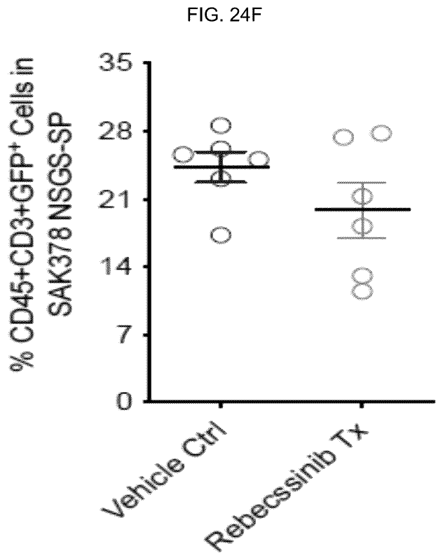

FIG. 24A-B illustrate cell sorting (FACS) scans illustrating levels of CD45+GFP+ cells after rebecsinib treatment;

FIG. 24C-D illustrate cell sorting (FACS) scans illustrating levels of CD45+CD3+GFP+ cells after rebecsinib treatment;

FIG. 24E graphically illustrates data from the cell sorting (FACS) scans illustrated in FIG. 24A-B; and

FIG. 24F graphically illustrates data from the cell sorting (FACS) scans illustrated in FIG. 24C-D.

### Example 13: Rebecsinib Treatment (tx) Spares Human CD45+ADAR1−GFP+ Cells and CD45+CD3+ADAR1−GFP+ Cells in aNBM SAK378 PDX Mouse BM

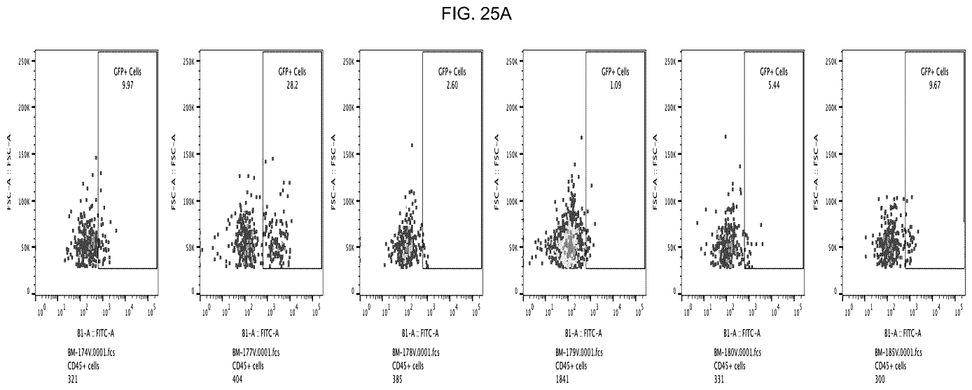

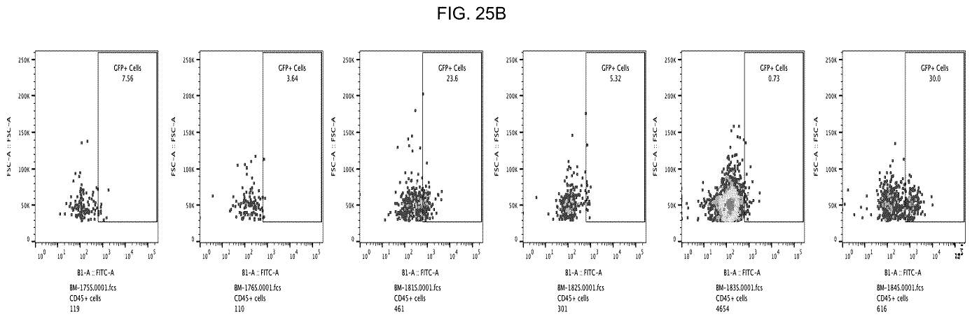

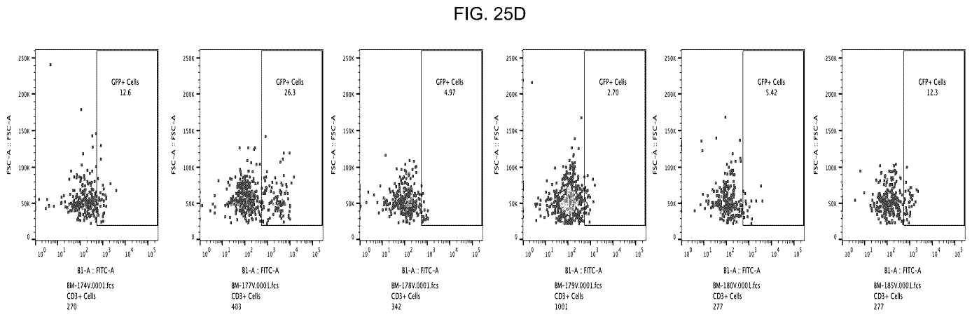

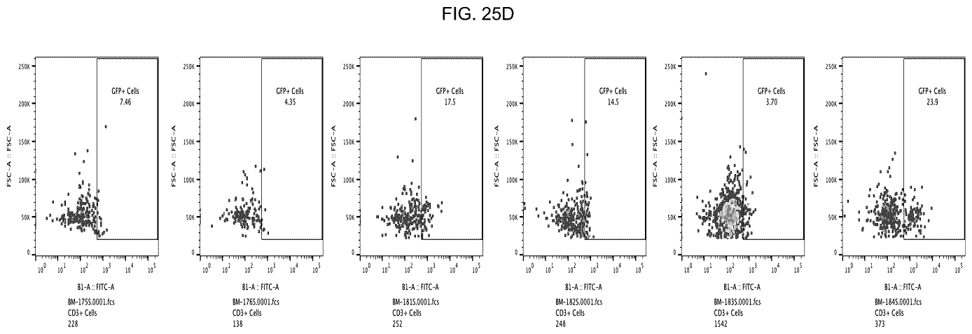

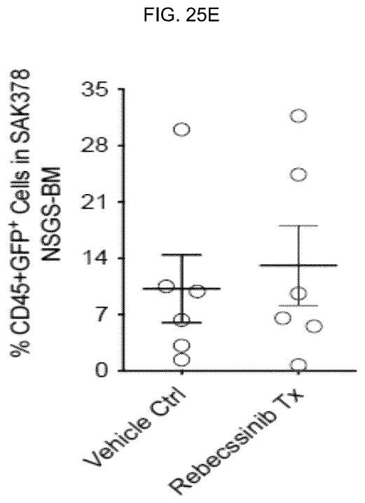

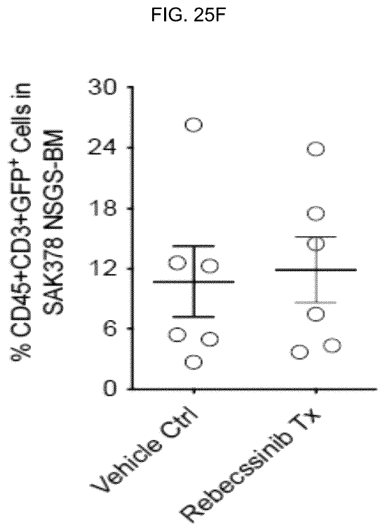

FIG. 25A-F graphically illustrate data demonstrating that rebecsinib treatment (tx) spares human CD45+ADAR1−GFP+ cells and CD45+CD3+ADAR1−GFP+ cells in aNBM SAK378 PDX Mouse BM:

FIG. 25A-B illustrate cell sorting (FACS) scans illustrating levels of CD45+GFP+ cells after rebecsinib treatment;

FIG. 25C-D illustrate cell sorting (FACS) scans illustrating levels of CD45+CD3+GFP+ cells after rebecsinib treatment;

FIG. 25E graphically illustrates data from the cell sorting (FACS) scans illustrated in FIG. 25A-B; and

FIG. 25F graphically illustrates data from the cell sorting (FACS) scans illustrated in FIG. 25C-D.

### Example 14: Reversal of Malignant ADAR1 Splice Isoform Switching with Rebecsinib

Adenosine deaminase acting on RNA1 (ADAR1) preserves genomic integrity by preventing retroviral integration and retrotransposition during stress responses. However, inflammatory microenvironment-induced ADAR1p110 to p150 splice isoform switching drives cancer stem cell (CSC) generation and therapeutic resistance in 20 malignancies. Previously, predicting and preventing ADAR1p150-mediated malignant RNA editing represented a significant challenge. Thus, we developed lentiviral ADAR1 and splicing reporters for non-invasive detection of splicing-mediated ADAR1 adenosine to inosine (A-to-I) RNA editing activation; a quantitative ADAR1p150 intracellular flow cytometric assay; a selective small molecule inhibitor of splicing-mediated ADAR1 activation, Rebecsinib, which inhibits leukemia stem cell (LSC) self-renewal and prolongs humanized LSC mouse model survival at doses that spare normal hematopoietic stem and progenitor cells (HSPCs); and pre-IND studies showing favorable Rebecsinib toxicokinetic and pharmacodynamic (TK/PD) properties. Together, these results lay the foundation for developing rebecsinib as a clinical ADAR1p150 antagonist aimed at obviating malignant microenvironment-driven LSC generation.

In this Example we describe the development of a lentiviral nanoluciferase-GFP (ADAR1 nanoluc-GFP) reporter that enables real-time, non-invasive detection of ADAR1-specific A-to-I RNA editing in human stem and progenitor cells as well as a lentiviral dual fluorescence (GFP/RFP) splicing reporter; and RNA-seq GRCh38-aligned computational bioinformatics platforms for quantifying ADAR1 splice isoform switching in normal human HSPCs, MPN HSPCs and LSCs. In addition, we have developed a flow cytometric assay for quantifying stem and progenitor cell ADAR1p150 protein expression levels and a selective small molecule inhibitor of splicing-mediated ADAR1 activation, Rebecsinib (17S-FD-895). In completed pre-IND studies (PIND 153126), Rebecsinib prevents ADAR1p150-splice isoform expression and malignant A-to-I editing-mediated LSC self-renewal at doses that spare normal HSPCs and are well tolerated in rat, rabbit, and non-human primate pre-IND toxicokinetic, PK and PD studies. Thus, clinical development of Rebecsinib may obviate A-to-I editing driven therapeutic resistance and reduce relapse-related mortality rates in AML and 20 therapeutically recalcitrant, ADAR1p150 overexpressing malignancies.

### Results

### Transcriptomic Detection of ADAR1 Splice Isoform Switching

Inflammatory cytokine-induced splice isoform switching of ADAR1 into the highly active A-to-I editing isoform, ADAR1p150, promotes solid tumor progression and drives high-risk MF hematopoietic progenitor cell (HPC) transformation into LSC.^{7-9,15,16,21-25} To determine the impact of ADAR1 splice isoform switching on LSC generation, we performed comparative whole transcriptome sequencing (RNA-seq) analyses of hematopoietic stem cells (HSCs) and HPCs from 1) healthy young and aged bone marrow samples, 2) myeloproliferative neoplasms, including polycythemia vera (PV), essential thrombocythemia (ET) and myelofibrosis (MF), and 3) chronic myeloid leukemia (CML) and AML samples (Table 1). Based on our previous observations that ADAR1-mediated RNA editing and splicing alterations occurs predominantly in the self-renewing CD34⁺CD38⁺Lin⁻ HPC population in sAML,^{5,8,9,14} we focused on this subpopulation for analysis of RNA editing and splicing changes in MPN pre-LSC and LSC. By evaluating RNA-seq pipelines and data based on GRCh38 (hg38) human genomic assembly,⁵ which identifies spliced junctions more reliably than hg19,²⁶ we observed increased expression of both the standard ADAR1p150 splice isoform, ADAR-202 (GRCh38 transcript ID ENST00000368474.9), and a recently identified ADAR1p150 isoform, ADAR-208 (ENST00000529168.2). The ADAR-208 isoform, which has a truncated 3′UTR that is predicted to prevent microRNA-mediated degradation, was enriched in MPN HPCs compared to normal young and aged bone marrow HPCs, along with the ADAR-202 isoform, while the ADAR-201 isoform was downregulated (FIG. 1A). This is in line with our previous observations that changes in ADAR1p150 occur predominantly in the malignant progenitor population and may reflect its central role in disease progression.⁹ This observation that a splice isoform switch may favor ADAR1p150 production led to our testing of the capacity of Rebecsinib (17S-FD-895), which binds within the spliceosome core complex (FIG. 1B),²⁷ to induce intron retention and to prevent splicing mediated ADAR1 BE activation (FIG. 1C).

### A Real-Time Lentiviral ADAR1 Reporter for Non-Invasive Detection of A-to-I RNA Editing

Key challenges in the CSC field include the capacity to reliably predict pre-CSC evolution to self-renewing CSCs and to detect tumor immune microenvironmental (TIME) drivers of dormant CSC maintenance, CSC immune evasion, and CSC therapeutic resistance, such as ADAR1. To date, the standard approach for quantifying ADAR1-mediated A-to-I base editing has relied on complex and cumbersome RNA-seq analyses. Moreover, the cell type and context-dependency of ADAR1 activation combined with the rapid degradation of inosine-containing transcripts, necessitates the development of a non-invasive live-cell detection system to accurately quantify real-time niche-dependent RNA base editing in normal stem cell, pre-CSC and CSC populations. Because lentiviral vectors sustainably integrate into the genomes of dormant stem cells, we chose to develop a lentiviral non-invasive reporter that selectively responds to ADAR1 activation for use in primary normal hematopoietic stem and progenitor cells (HSPCs), MF HPCs and LSCs both in vitro and in vivo.

To develop an ADAR1-specific lentiviral A-to-I base editing reporter, we incorporated an ADAR1-specific synthetic nucleic acid sequence²⁸ into a stem cell promoter-driven (EF1a) pCDH lentiviral vector that enables rapid detection of A-to-I editing. Specifically, ADAR1-mediated A-to-I RNA base editing removes a stop codon within the synthetic sequence and induces downstream nanoluciferase and GFP (ADAR1 nanoluc-GFP) expression (FIG. 2A). We then overexpressed this vector in combination with a lentiviral ADAR1 overexpression vector that recapitulates endogenous induction of the interferon-responsive ADAR1p150 isoform in human TF-1a ANIL cells. In contrast to catalytically inactive ADAR1 (E912A) mutant or ADAR2 proteins, the lentiviral ADAR1 nanoluc-GFP reporter showed a dose dependent increase in A-to-I base editing (BE) activity that responded to overexpression of wild-type ADAR1p150, further highlighting the specificity and sensitivity of our BE reporter system (FIGS. 2B and C). This system represents an important advance over recently described BE reporter and sensor assays that are not selective between ADAR1 and ADAR2 activity,^{29,30} due to the functional implications of ADAR1-mediated RNA editing in cancer and stem cell biology. The lentiviral ADAR1 nanoluc-GFP reporter could be detected in human leukemia cells in vitro by confocal fluorescence microscopic detection of GFP (FIG. 2D) and by non-invasive (IVIS, Caliper) bioluminescence detection of ADAR1 BE activity in mice engrafted with human leukemia cell lines (K562 and TF-1a) expressing wild-type ADAR1 compared with no transplant and lentiviral pCDH backbone or ADAR1 E912A mutant controls (FIG. 2E). These studies confirmed the specificity and sensitivity of our lentiviral ADAR1 BE reporter both in vitro and in vivo.

Moreover, in a manner that phenocopied ADAR1 shRNA knockdown in a microenvironmentally-responsive TF1a AML cell line, Rebecsinib treatment reduced ADAR1 expression as measured by qRT-PCR, with low levels of AZIN1 transcript editing activity as shown by RNA editing site-specific qPCR (RESSqPCR) detected in shADAR1-transduced cells treated with Rebecsinib compared with vehicle controls. Moreover, stromal co-cultures assays performed with primary high-risk myelofibrosis (MF) pre-LSC (MF HPCs), which were lentivirally transduced with the lentiviral ADAR1 nano-Luc-GFP reporter, revealed that Rebecsinib treatment significantly reduced ADAR1 RNA editing activity (FIG. 2F). Because ADAR1 induces A-to-I intronic editing and upregulation of STAT3 isoforms, which transcriptionally activate ADAR1,^{5,8,16} we assessed the effects of Rebecsinib on phospho-STAT3 expression. In keeping with inhibition of ADAR1 base editing activity, Rebecsinib reduced phospho-STAT3 expression, as shown by phospho-STAT3 intracellular flow cytometry (FIG. 2G). Together, these studies provided the rationale for testing the high-risk MF HPC and LSC inhibitory efficacy of Rebecsinib in survival and self-renewal assays with multiple primary patient samples (Table 1).

### Inhibition of ADAR1p150 Activation Prevents High-Risk MF HPC and LSC Maintenance

To quantify ADAR1 protein expression in HSPCs and LSCs, we developed a flow cytometric assay with HSPC and LSC cell surface markers and an ADAR1p150-specific antibody. Compared with vehicle controls, treatment of stromal co-cultures with Rebecsinib (FIG. 3A) decreased MF HPC viability commensurate with reduced ADAR1p150 protein expression as detected by intracellular flow cytometry (FIGS. 3B and C). Both clonogenic survival and replating (self-renewal) assays demonstrated greater sensitivity of secondary AML (sAML) LSC to Rebecsinib than MF HPC or normal cord blood, young bone marrow or aged bone marrow HPCs, independent of splicing factor mutational status in sAML (FIGS. 3D and E. In contrast to MF HPC and LSC, no significant differences were detected in normal HSPC and mature hematopoietic progeny exposed to doses of Rebecsinib that decreased LSC survival and self-renewal in co-culture assays (1 μM) compared with DMSO (vehicle) treated controls. These data provide evidence for a favorable therapeutic index with Rebecsinib.

### Splicing Reporter and Splice Isoform Biomarkers of Rebecsinib Response

In addition to testing the effects of Rebecsinib in lentiviral ADAR1 nanoluc-GFP activity assays, we also confirmed its activity using a lentiviral dual fluorescence splicing reporter that shows increased RFP compared with GFP expression upon splicing modulation. The design of this reporter was based on a non-lentiviral reporter construct that was previously tested with Rebecsinib but that was not compatible with use in primary human HSPC.^{14,31} Cloning into a lentiviral construct allows confocal microscopic imaging, in vivo imaging of fluorescence (IVIS Caliper), and flow cytometric quantification of splicing activity using the ratio of RFP to GFP signals in primary cells. Rebecsinib impaired CD34⁺ KG-1a AML cell survival and increased RFP to GFP reporter expression in a dose-dependent manner, with an IC50 dose of approximately 0.1 μM (FIG. 4A). In primary patient sample-derived CD34⁺ MF HPC and LSC short-term culture with Rebecsinib (no stroma), increased exon skipping resulted in elevated expression of pro-apoptotic MCL1-short (S) compared with anti-apoptotic MCL1-long (L) transcripts (expressed as ratios of MCL1-S/L) in all samples treated with 0.1 μM Rebecsinib compared with vehicle (FIGS. 4B and C). Moreover, Rebecsinib treatment phenocopied ADAR1 shRNA knockdown, with respect to repression of ADAR1 activity, and reduced expression of LSC-associated transcripts, such as CD44v3¹³ and MCL1-L. Moreover, ADAR1 shRNA knockdown reduced MCL1-L expression, which is consistent with a previous report involving human cancer cell lines where ADAR1 knockdown attenuates STAT3 activity and subsequent MCL1 transcription.³² These studies suggest that Rebecsinib inhibits splicing-mediated ADAR1 activation which is a central driver of LSC self-renewal,^{5,9,16} and is potentiated by MCL1 splicing modulation.¹²

### Rebecsinib Pharmacokinetic, Toxicokinetic and Pharmacodynamic Pre-Clinical Studies

To facilitate pharmacodynamic (PD) studies in rats, rabbits and non-human primates (NHP), we developed and validated a panel of functionally relevant species-specific splice isoform biomarker primers to detect responses to Rebecsinib. Based on our current and previous work identifying splice isoform biomarkers of molecular response to splicing modulation,^{14,33} the transcripts selected for species-specific primer design included SF3B family members and the AML LSC-signature transcript, PTK2B-202, along with the pro-survival gene, MCL1, and the LSC self-renewal driver ADAR1p150 (FIGS. 3 and 4). Three unique human AML cell lines, including KG-1a, MOLM-13 and HL-60, were tested for response to Rebecsinib treatment at a final concentration of 1 μM with significant upregulation of intronic retention of SF3B family members, with SF3B3 demonstrating the most potent and consistent response across all cell lines. Decreased expression of the LSC-related PTK2B-202 isoform was also observed following treatment with Rebecsinib. In rat RBL-1 leukemia cells, intron retention in Sf3b family members was rapidly induced after treatment with different lots of Rebecsinib. Together, these biomarkers will be used to monitor responses to Rebecsinib treatment in IND-enabling studies.

Pre-IND PD and toxicokinetic (TK) studies (FIG. 4D) were enabled by scalable Rebecsinib synthesis³⁴ and development of an optimized formulation for in vivo biodistribution (5% w/v EtOH, 5% w/v Kolliphor HS15 in 0.9% Sodium Chloride). In PK studies, Rebecsinib was detectable in rat, rabbit, and non-human primate (NHP) plasma following IV bolus administration. In rat TK studies, Rebecsinib was quantifiable up to 1-hour post-dose at 1 mg/kg and up to 4 hours post-dose at 3, 5, and 8 mg/kg (FIG. 4E). Rebecsinib Tₘₐₓ values were observed by 0.083 hours post-dose at 1 and 5 mg/kg and by 0.167 hours post-dose at 3 and 8 mg/kg. Rebecsinib T_{1/2} values were 0.342, 0.447, 0.313, and 0.530 hours at 1, 3, 5, and 8 mg/kg, respectively. In rabbit TK studies, plasma t_{1/2} values ranged from 0.12 to 0.89 hours. Following 3, 10, and 20 mg/kg boluses, Rebecsinib was quantifiable in plasma 1 hour post-dose and could be detected up to 8 hours post-dosing with 40 mg/kg (FIG. 4F). In NHP TK studies, peak (Cₘₐₓ) and total (AUCₗₐₛₜ) levels following exposure to Rebecsinib were comparable between male and females at all dose levels (FIG. S3C). Following 3, 10, 15, and 20 mg/kg of Rebecsinib, quantifiable plasma concentrations of Rebecsinib were observed through 8 hrs post-dose (FIG. 4G). Rebecsinib half-life (t_{1/2}) values ranged from 0.62 to 1.1 hours. While one female NHP in the 20 mg/kg treatment group developed diarrhea, it resolved within 24 hours with no treatment and no sequelae indicative of favorable tolerability.

Pharmacodynamic studies were conducted on peripheral blood mononuclear cells (PBMCs) isolated from NHPs treated with escalating doses of Rebecsinib (3 mg/kg, 10 mg/kg, 15 mg/kg or 20 mg/kg) or a vehicle control and collected at 30 minute and 4 hours post-dose. Splice isoform-specific qRT-PCR demonstrated on-target splicing modulation typified by MCL1 exon skipping and SF3B3 intron retention following Rebecsinib dosing (FIG. 4H). Compared with vehicle, SF3B3 intron retention levels increased at 30 min following single Rebecsinib doses of 3, 10, 15 or 20 mg/kg and were detectable at 4 hours. Taken together, TK studies show favorable characteristics and PD analyses demonstrate predictable, dose-dependent splicing modulation with Rebecsinib.

### Rebecsinib-Mediated Inhibition of ADAR1 Splicing Reduces LSC Self-Renewal

To evaluate the inhibitory efficacy of Rebecsinib, we performed in vivo humanized LSC mouse model serial transplantation assays, as a gold-standard measurement of self-renewal of the malignant progenitor (pre-LSC and LSC) population, as well as survival assays (FIG. 5A).¹⁴ With a twice weekly dosing regimen, there was a significant reduction in sAML LSC burden in the bone marrow, peripheral blood, and spleen of Rebecsinib-treated mice engrafted with splicing factor mutated and unmutated primary AML patient samples. There was a concomitant increase in the pro-apoptotic MCL1-S isoform expression in human CD34⁺ cells isolated from the bone marrows of these mice.

In purified human CD34⁺ cells derived from Rebecsinib-treated sAML50261 engrafted mice, there was a significant reduction in total ADAR1 RNA expression by qRT-PCR as well as LSC-specific splice isoform biomarkers, including CD44-012 and PTK2B-202, compared with vehicle treated controls. Moreover, RNA-seq-based analyses of ADAR1 transcript splicing revealed unique retained intron regions and alternative splice site usage in Rebecsinib-treated samples. In Rebecsinib-treated sAML50261 engrafted mice, intracellular flow cytometry assays detected significantly decreased ADAR1p150 protein isoform levels in both human HSC and HPC compared with vehicle controls. A comprehensive investigation of the molecular changes after in vivo Rebecsinib treatment from our previous datasets¹⁴ revealed a global downregulation of RNA editing activity that occurred in a dose-responsive manner, concomitant with splice isoform switching of MCL1-L to MCL1-S, in mice treated with 5 or 10 mg/kg of Rebecsinib. In comparative in vivo studies using fedratinib, a small molecule JAK2 inhibitor that modulates ADAR1 expression by inhibiting its transcriptional activation by STAT3, we found no alteration of MCL1 splicing but, as expected, we observed significantly reduced ADAR1 expression. In contrast, Rebecsinib inhibits both MCL1-L pro-survival transcript expression and ADAR1p150-mediated RNA editing suggesting that it has the capacity to impair LSC survival and self-renewal.

To test this, we performed serial transplantation assays of cells harvested from sAML50261 LSC engrafted mice that were treated intravenously twice weekly for two weeks with Rebecsinib at 10 mg/kg (serial transplant recipients received no further treatment). Flow cytometry analyses of human hematopoietic cell engraftment confirmed that there was a reduced frequency of hematopoietic progenitors (CD34⁺CD38⁺Lin⁻) in the spleens of serial transplant recipients of cells isolated from treated mice. Serial transplantation after treatment of a splicing factor mutated sAML (2008-5) engrafted mouse model confirmed that Rebecsinib treatment significantly reduced LSC self-renewal. In a separate cohort, mice that received CD34⁺ cells from Rebecsinib-treated splicing factor unmutated sAML 50261 engrafted mice displayed a significant improvement in overall survival, indicative of a significant reduction in LSC self-renewal capacity. Intriguingly, further molecular analysis of serially transplanted cells harvested from these mice revealed that a less well-characterized interferon-responsive transcript of ADAR1, ADAR-208, which encodes for ADAR1p150 but has a truncated 3′UTR and is lacking a short region of the dsRNA binding domain, shows sustained reductions in expression after serial transplantation compared with ADAR1p110. Thus, reductions in ADAR1p150-mediated RNA editing activity following Rebecsinib treatment correspond with a reversion to a healthy splice isoform expression profile¹⁴ in engrafted human progenitors. Together, these results demonstrate that Rebecsinib reduces both the survival and self-renewal potential of sAML LSC in primary patient-derived splicing factor-mutated and unmutated LSC engraftment models, and promotes improved mouse survival in serial transplantation assays. These results demonstrate that Rebecsinib-mediated inhibition of ADAR1p150 splice isoform expression and activity impairs LSC self-renewal in vivo.

### Normal Human HSPCs Exhibit Functional Resistance to Rebecsinib

To determine the sensitivity of normal HSPCs to Rebecsinib, human cord blood-derived CD34+ cells were transplanted into Rag2^{-/-}γc^{-/-} mice. After human cell engraftment was established (6 to 12 weeks), mice were treated with Rebecsinib twice-weekly for two weeks at doses equivalent to the maximum doses selected for in vivo LSC assays. The frequency of CD45⁺ hematopoietic cells in bone marrow, peripheral blood, spleen, and thymus were unchanged between vehicle and Rebecsinib-treated groups that received a dose of 10 mg/kg. Moreover, flow cytometric analyses showed no significant reduction in human CD3⁺ T cells in thymus or peripheral blood samples or in CD19⁺ B cells in the bone marrow, peripheral blood, or spleen of mice treated with Rebecsinib compared with vehicle controls. In mice treated with 20 mg/kg of Rebecsinib, there was a reduction in human CD45⁺ and CD19+ cells in the peripheral blood cell engraftment but not in the bone marrow or spleen. Thus, a dose of 10 mg/kg in mice is sufficient to reduce in vivo sAML LSC burden while sparing normal HSPC development. In addition, no evidence of Rebecsinib-related systemic toxicity was observed after 2 weeks of dosing at 10 or 20 mg/kg in this humanized in vivo model of normal HSPC development.

After completion of treatment and engraftment analyses of in vivo normal HSPC assays, total human hematopoietic cells (CD45⁺) were selected for splice biomarker analyses to determine sensitivity of normal human hematopoietic cells to Rebecsinib compared with sAML LSC. These analyses showed no changes in SF3B3 intron retention in the bone marrow but a trend toward changes in the spleen that were not statistically significant. Together, our humanized mouse model assays combined with our splice isoform biomarker system confirms a functional and molecular therapeutic index for Rebecsinib, whereby sAML LSC are significantly more sensitive to splicing modulation than normal HSPCs and their progeny.

### Sensitive Detection of ADAR1 Activity in Primary Human Cells In Vivo

To further test the utility of the ADAR1 nanoluc-GFP reporter for in vivo detection of endogenous ADAR1 activity, primary human aged normal bone marrow CD34⁺ cells were transduced with the reporter vector and transplanted into immunocompromised mice. ADAR1 activity was detected by live animal bioluminescence imaging and corresponded with human cell engraftment detection by flow cytometry. Moreover, treatment of this normal aged HSPC in vivo model with Rebecsinib at 10 mg/kg confirmed that healthy human hematopoietic cells tolerate splicing modulation, with no loss of human HSPC engraftment or mature T or B cell maturation. In comparison to cord blood transplantation models, the frequency of normal CD34+ cells appeared to be increased in the bone marrow of Rebecsinib-treated animals. This suggests that Rebecsinib spares normal HSPC survival and retention in the bone marrow niche, which is the subject of ongoing mechanistic studies. Together, these results further support a favorable therapeutic index of Rebecsinib that has been observed in comprehensive in vitro and in vivo IND-enabling studies.

### Discussion

In sAML, 5-year survival rates of only 26%, are fueled, at least in part, by therapy resistant LSC survival and self-renewal.³⁵ Over 50% of patients succumb to AML in the first year (10,590 deaths out of 21,380 new cases in 2017³⁶) and mortality rates have remained similar over four decades thereby underscoring the pressing unmet need for selective LSC inhibitors. Despite advances in molecularly targeted therapy, morbidity and mortality rates also remain elevated for patients with high-risk MF.³⁷ Inflammatory microenvironment-induced adenosine-to-inosine (A-to-I) hyper-editing by ADAR1 has been linked to therapeutic resistance in sAML, MF, chronic myeloid leukemia, multiple myeloma and 14 different solid tumor types.^{5,7-9,15,16,23} Also, recent studies show that splicing deregulation drives therapeutic resistance and that inflammatory cytokine induced A-to-I RNA editing introduces novel splice acceptor sites, such as in STAT3, which promote LSC generation.^{14,38-40} Previously, we showed that LSC splicing deregulation was associated with increased sensitivity to the splicing modulator, 17S-FD-895 (Rebecsinib). However, a mechanistic link between A-to-I editing activation by ADAR1 and splicing deregulation had not been established.

To determine if niche-dependent splicing deregulation drives ADAR1 activation, we developed sensitive lentiviral ADAR1 nanoluciferase GFP and lentiviral dual fluorescence splicing reporters that enable non-invasive imaging, confocal fluorescence microscopic detection and flow cytometric quantification of ADAR1 activation and splicing deregulation in selective microenvironments. In addition, we developed ADAR1 shRNA knockdown and ADAR1p150 overexpression vectors for use in normal HSPC, MF HPC and LSC human SCF, IL-3 and GM-CSF secreting stromal co-cultures and humanized mouse model systems. By demonstrating that genetic inhibition of ADAR1 expression with lentiviral shRNA knockdown vectors phenocopies pharmacologic splicing inhibition with Rebecsinib, we discovered that A-to-I hyper-editing is predicated on ADAR1 splice isoform switching favoring ADAR1p150 over ADAR1p110.

Moreover, we found that ADAR1p150 inhibition with Rebecsinib was well tolerated at doses that eradicated LSC and spared normal HSPC. As a result of ADAR1 induced self-renewal dependency, Rebecsinib treatment inhibited sAML LSC replating and serial transplantation and enhanced survival of humanized sAML mouse models commensurate with dose-dependent changes in ADAR1p150 transcript and protein expression as well as decreased A-to-I editing activity. These in vitro and in vivo studies provided a mechanistic link between microenvironmentally-driven splicing deregulation and ADAR1-mediated A-to-I editing activation in LSC in sAML and potentially in other ADAR1-activated malignancies.²⁴ In addition to preventing ADAR1 transcript processing, Rebecsinib reduces LSC-enriched anti-apoptotic transcripts, including MCL1-L, BCL-XL, and BCL-2, and induces marked intron retention in splicing factor gene products, such as SF3B1 and SF3B3, which form part of the splicing modulator binding pocket.

In multi-species (rat, rabbit, NHP) pre-IND studies, Rebecsinib was well tolerated and induced dose-dependent increases in splicing modulation. With a clinically tractable formulation, Rebecsinib showed predictable pharmacokinetic and pharmacological (PK/PD) properties combined with favorable bioavailability and stability thereby enabling twice-weekly intravenous dosing regimens with no evidence of systemic toxicity.

The high likelihood of clinical feasibility with Rebecsinib is based on extensive pre-clinical studies showing chemical stability, toxicokinetic safety, favorable pharmacokinetic and pharmacodynamic properties, and highly efficacious human MF HPC and LSC-targeting capacity in splicing factor-mutated and unmutated humanized models of sAML. While one chemically distinct splicing modulator, H3B-8800, completed Phase 1 clinical trials for hematologic malignancies resulted in red blood transfusion independence in some patients, its efficacy was dependent on SRSF2 mutations and it was not sufficiently potent to induce durable remissions.⁴¹ Another splicing modulator, E7107, induced reversible optic neuritis in 2 of 26 patients with solid tumors,^{42,43} which was related to compound instability⁴⁴ and resulted in early clinical trial discontinuation. In contrast, Rebecsinib has a favorable potency profile and therapeutic index compared to other splicing modulators. Overall, this study lays the foundation for clinical development of Rebecsinib as an ADAR1 self-renewal pathway inhibitor aimed at obviating LSC driven therapeutic resistance and relapse in patients with high-risk MF and sAML and potentially for other malignancies that resist immune checkpoint blockade as a result of ADAR1-mediated immune silencing.¹⁹

As a potent first-in-class ADAR1 inhibitor, Rebecsinib decreases RNA editing by inhibiting ADAR1 splicing into the most active editase, ADAR1p150. While active in high-risk MF HPC and LSC, a limitation of the current study is that we did not examine the dependence of solid tumor CSC self-renewal on splicing mediated ADAR1 activation and sensitivity to Rebecsinib-mediated ADAR1 splicing inhibition. For future IND enabling studies with Rebecsinib, which are beyond the scope of this completed pre-IND proof-of-concept study, we have developed lentiviral ADAR1 reporter expressing tumor organoid-containing nanobioreactors that enable niche-dependent detection of A-to-I editing activation and humanized CSC ADAR1 reporter models. The capacity of Rebecsinib to potently inhibit malignant A-to-I editing may enhance the spectrum of therapeutically sensitive malignancies to include 14 solid tumor types that activate ADAR1.⁴¹

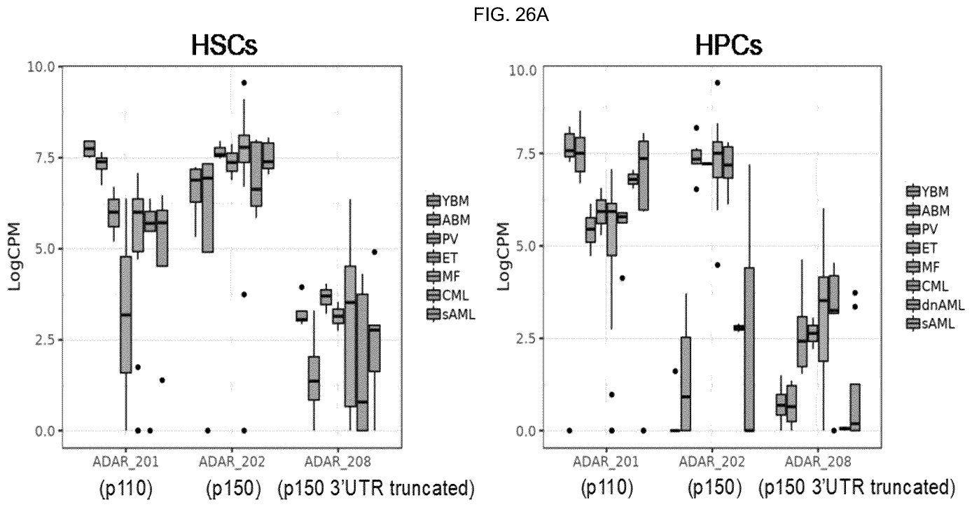

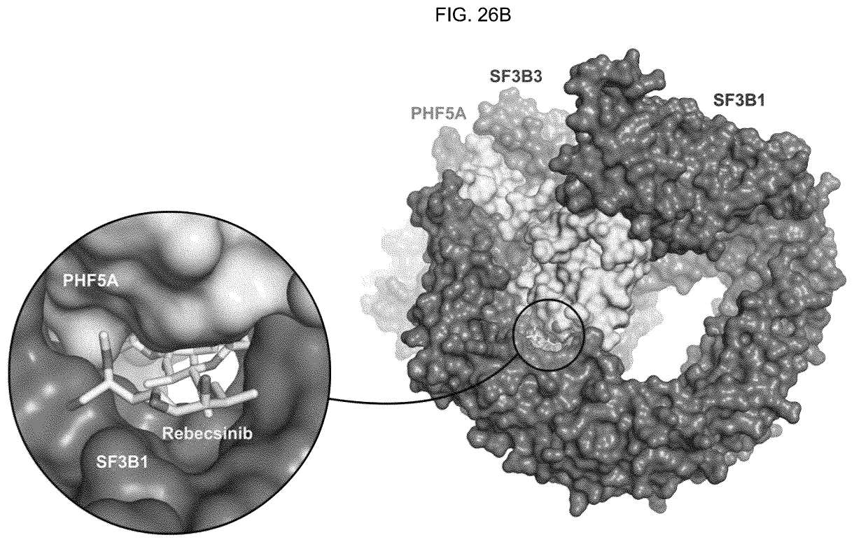

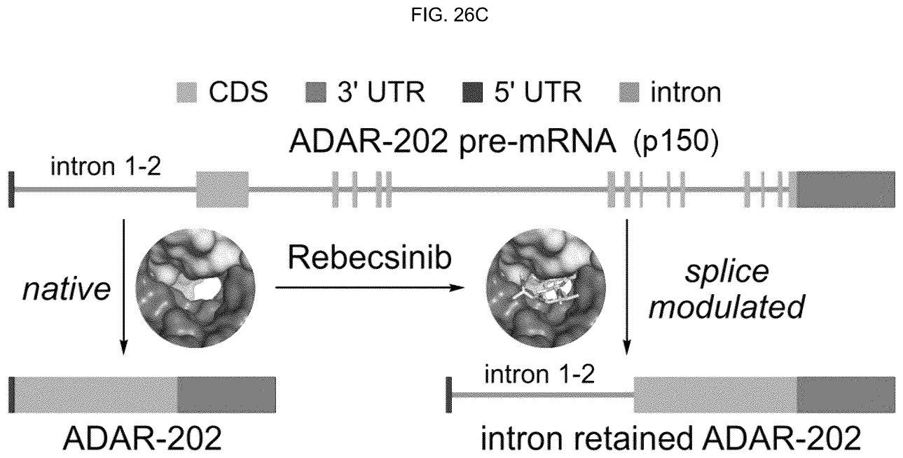

### FIG. 26A-C (or FIG. 1 of Example 14): Quantification of ADAR1p150 by Splice Isoform RNA Sequencing (RNA-Seq)

(FIG. 26A) RNA-seq-based quantification (counts per million, CPM) of ADAR-201 (GRCh38 ENST00000368471.8, ADAR1 p110-encoding), ADAR-202 (ENST00000368474.9, ADAR1 p150-encoding), and ADAR-208 (ENST00000529168.2, ADAR1 p150-encoding 3′UTR truncated transcripts) was performed on FACS-purified hematopoietic stem cells (HSC, CD34⁺CD38⁻Lin⁻) from young (YBM; n=4) and aged bone marrow (ABM; n=4) HSC, polycythemia vera (PV, n=3), essential thrombocythemia (ET, n=2), myelofibrosis (MF, n=24), chronic myeloid leukemia (CML, n=5), or secondary acute myeloid leukemia (sAML, n=5). RNA-seq analyses were also performed on FACS-purified hematopoietic progenitor cells (HPC, CD34⁺CD38⁺Lin⁻) from primary samples, including YBM (n=8), ABM (n=8), PV (n=6), ET (n=2), MF (n=24), CML (n=5), de novo (dnAML and sAML) (n=13) AML. Statistics for HSC: ADAR-201 p<0.05 for MF, CML, and sAML versus ABM; ADAR-202 p<0.05 for MF versus ABM; ADAR-208 differences were not significant in HSC. Statistics for HPC: ADAR-201 p<0.05 for PV, ET, MF, and CML versus ABM; ADAR-202 p<0.05 for PV, ET, MF, and CML versus ABM; ADAR-208 p<0.05 for PV, ET, and MF versus ABM. Statistical analyses were performed using Student's t-tests comparing MPNs and sAML versus ABM.

(FIG. 26B) Structural diagram showing the spliceosome core complex with Rebecsinib interacting at the interface of SF3B1 and PHF5A, adapted from the spliceosome complex bound to pladienolide B.²⁷

(FIG. 26C) Schematic diagram of the primary ADAR1 p150-encoding transcript, ADAR-202, and proposed Rebecsinib-induced intron retention reducing transcript expression after treatment.

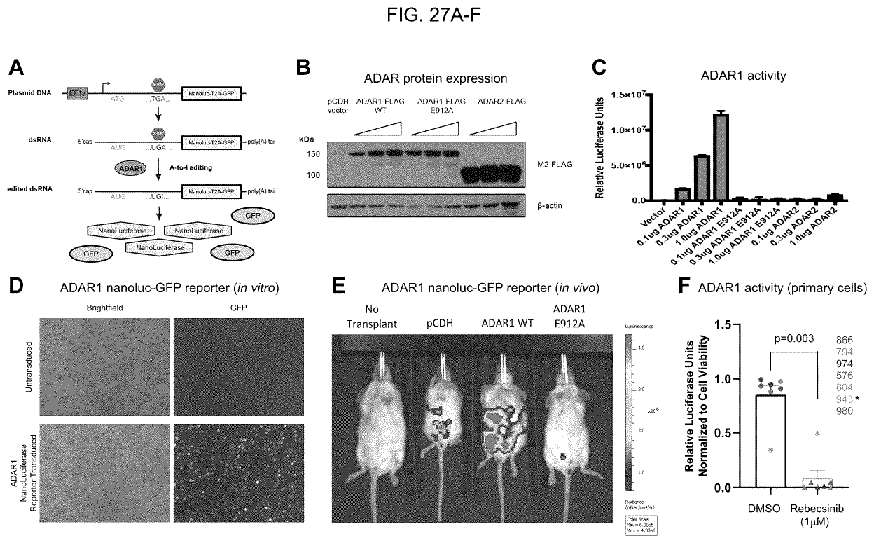

### FIG. 27A-G (or FIG. 2 of Example 14): Development of a Lentiviral ADAR1 A-to-I RNA Editing Reporter

(FIG. 27A) Schematic diagram demonstrating the synthetic RNA sequence containing an ADAR1-sensitive stop codon that, upon A-to-I editing, reads through to produce nanoluciferase and GFP proteins separated by a T2A cleavage site.

(FIG. 27B) ADAR protein expression levels in 293T cells co-transfected with the ADAR1 nanoluciferase-GFP (nanoluc-GFP) reporter and increasing amounts of FLAG-tagged wild-type (WT) ADAR1, catalytically inactive mutant ADAR1 (E912A), or wild-type ADAR2. β-actin was used as a loading control.

(FIG. 27C) Relative luciferase signals in 293T cells prepared as in panel B. Data are represented as mean±SEM.

(FIG. 27D) Live cell fluorescent imaging of GFP expression in human myeloid leukemia TF-1a cells transduced with the ADAR1 nanoluc-GFP reporter vector (lower panels) compared to untransduced controls (upper panels).

(FIG. 27E) Detection of nanoluciferase expression via in vivo bioluminescence (IVIS) imaging of no transplant control (far left), K562-nanoluc-GFP and pCDH vector transduced and K562-nanoluc-GFP and ADAR1 wild-type or E912A mutant transduced human leukemia cells (K562) transplanted into RAG2^{-/-}γc^{-/-} mice.

(FIG. 27F) Luminescence-based quantification of ADAR1-dependent nanoluciferase signals in CD34⁺ cells from primary, high-risk MF samples (*=untreated patient) after in vitro transduction with the ADAR1 nanoluc-GFP reporter and treatment with vehicle control (DMSO) or Rebecsinib (72 hr). Relative luciferase signals were normalized to cell viability for each condition. Data are represented as mean±SEM. p<0.05 compared to DMSO controls by pairwise t-test.

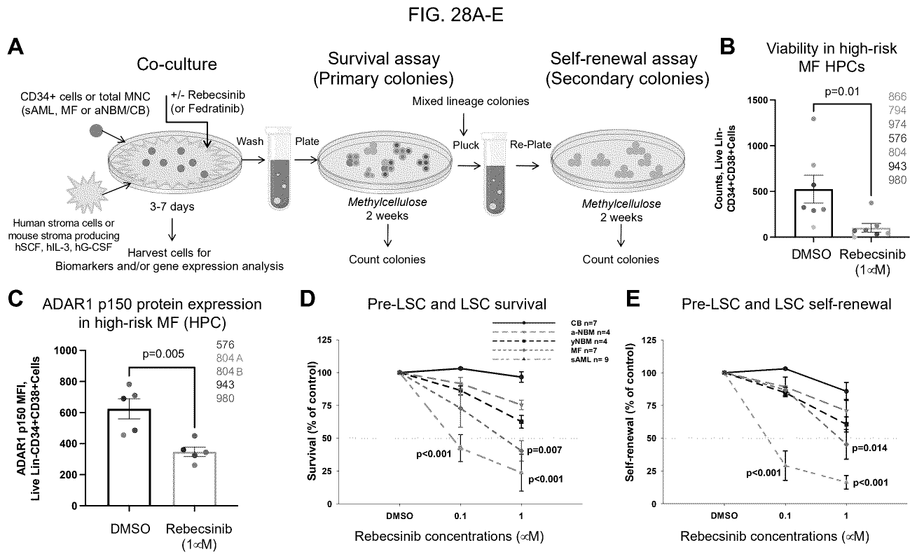

### FIG. 28A-E (or FIG. 3 or Example 14): Rebecsinib Inhibits ADAR1p150 Mediated High-Risk MF HPC and LSC Survival

(FIG. 28A) Schematic diagram of in vitro MF HPC and LSC survival and self-renewal assays.

(FIG. 28B) Flow cytometry-based viable cell counts (5,000 events measured) in high-risk MF samples after in vitro treatment of primary CD34⁺ cells with vehicle control (DMSO) or Rebecsinib (72 hr).

(FIG. 28C) Flow cytometry-based quantification of ADAR1 p150 protein expression in high-risk MF samples after in vitro transduction of primary CD34⁺ cells with ADAR1 nanoluc-GFP reporter or vector control (pCDH) followed by treatment with vehicle control (DMSO) or Rebecsinib (72 hr).

(FIG. 28D, FIG. 28E) Quantification of colony formation (survival, FIG. 28D) and replating (self-renewal, FIG. 28E) of high-risk MF HPC and sAML LSC compared with cord blood (CB) and aged versus young normal bone marrow (a-NBM, y-NBM) controls treated with Rebecsinib at increasing concentrations. Bar graphs show data as mean±SEM and statistical analyses by pairwise t-test and dose-response assays show data as mean±SD and statistical analyses by one-way ANOVA.

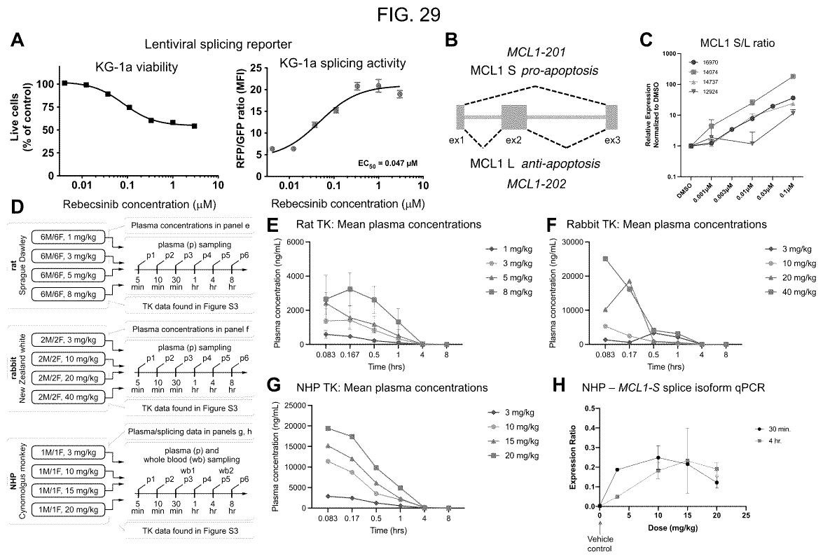

### FIG. 29A-H (or FIG. 4 of Example 14): Rebecsinib Pharmacodynamic and Pharmacokinetic Studies in Pre-Clinical and Pre-IND Models

(FIG. 29A) Quantification of cell viability (left panel) and splicing modulation (RFP/GFP ratios, right panel) by flow cytometry analyses of the human AML cell line (KG-1a) stably transduced with a lentiviral dual-fluorescence splicing reporter vector and treated with increasing concentrations of Rebecsinib.

(FIG. 29B) Transcript diagram illustrating alternative splicing of MCL1 to generate MCL1-short (S, pro-apoptosis) and MCL1-long (L, anti-apoptosis) variants. For human cells, the ratio of MCL1-short to long isoforms is shown.

(FIG. 29C) MCL1 S/L ratios in Rebecsinib dose response assays performed using primary sAML LSC (without stromal co-culture). Splice isoform-specific qRT-PCR values were normalized to DMSO-treated controls for each individual patient sample.

(FIG. 29D) Schematic diagram outlining multispecies toxicokinetic (TK) and pharmacodynamic studies in mammalian species treated in vivo with a single dose of Rebecsinib. Toxicokinetic analyses were performed in rats (n=6 per sex, per group), rabbits (n=2 per sex, per group), and non-human primates (NHPs, n=1 per sex, per group) and pharmacodynamic splice isoform quantification studies were performed in peripheral blood mononuclear cells (PBMCs) from NHP.

(FIG. 29E, FIG. 29F) For toxicokinetic analyses, rats (FIG. 29E) and rabbits (FIG. 29F) were given a single injection of Rebecsinib at 1-40 mg/kg, or vehicle control, and blood samples were drawn at regular intervals to determine plasma concentrations of the compound over 8 hr after treatment.

(FIG. 29G, FIG. 29H) For in vivo TK and complementary pharmacodynamic studies, NHPs were given a single injection of Rebecsinib at 3-20 mg/kg, or vehicle control, and blood samples were drawn at regular intervals to determine plasma concentrations (FIG. 29G) of the compound along with splice isoform biomarker assays to quantify MCL1 exon skipping in PBMCs isolated from treated animals (FIG. 29H, n=2 animals per group).

All data are represented as mean±SEM.

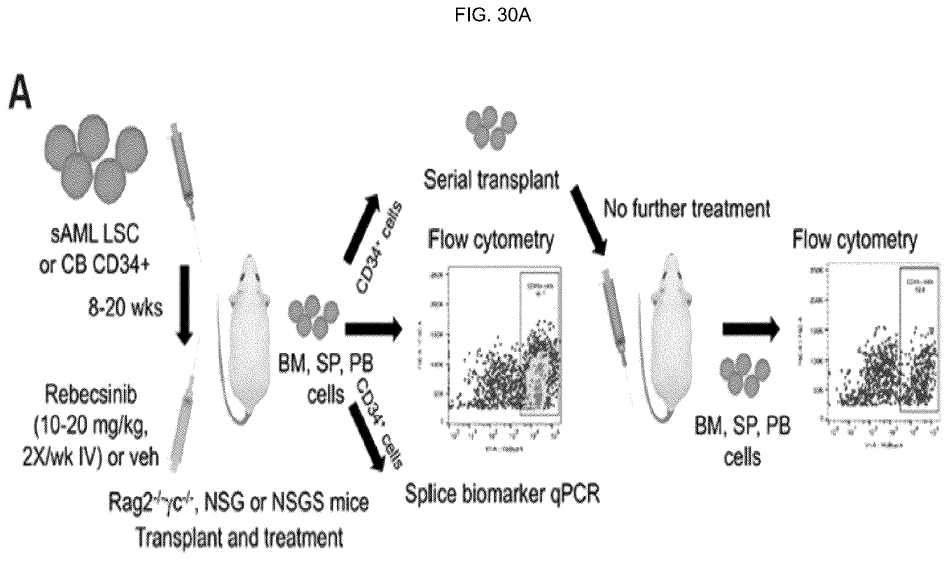

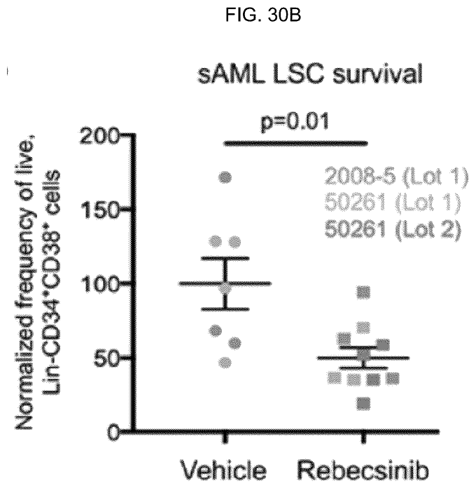

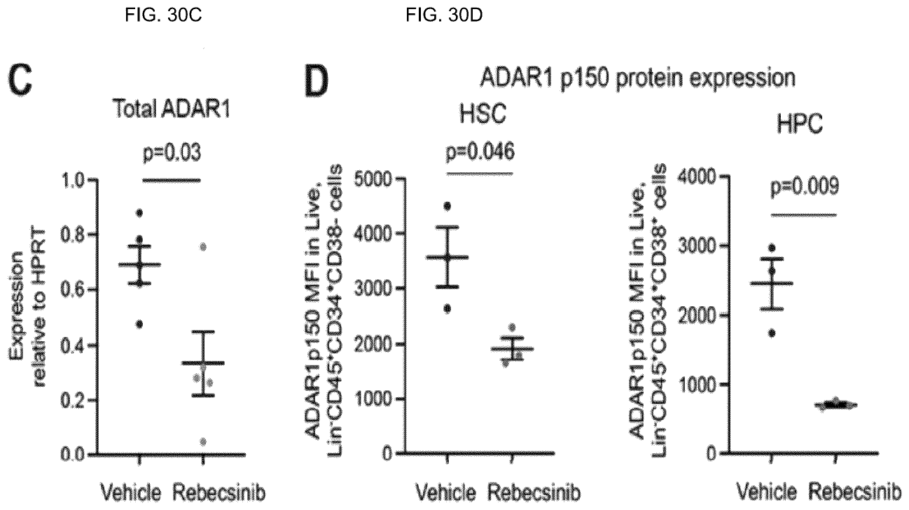

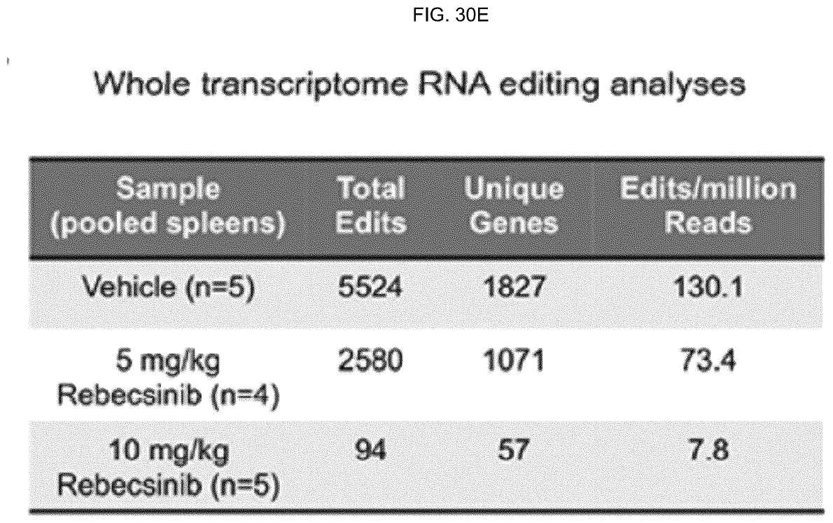

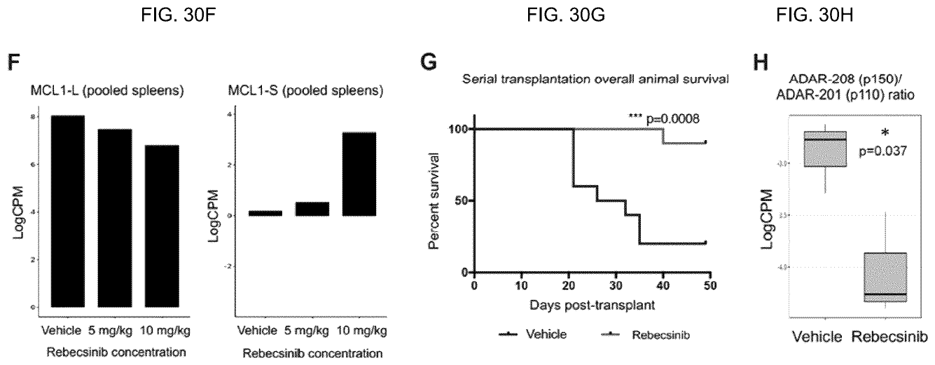

### FIG. 30A-H (or FIG. 5 of Example 14): ADAR1 Expression and LSC Self-Renewal Following Rebecsinib Treatment

FIG. 30A-H illustrate ADAR1 expression and LSC self-renewal following Rebecsinib treatment:

FIG. 30A illustrates a schematic diagram showing in vivo treatment of primary patient LSC or cord blood (CB)-engrafted mice and sAML serial transplantation studies;

FIG. 30B illustrates a flow cytometry analysis quantifying human LSC survival in sAML-engrafted mice treated with Rebecsinib Lot 1 or Lot 2 compared with vehicle control at 10 mg/kg twice weekly for two weeks;

FIG. 30C illustrates a qRT-PCR analyses in CD34⁺ cells isolated from the spleens of sAML50261 mice treated with Rebecsinib (as in A) showing decreased total ADAR1 expression by qRT-PCR;

FIG. 30D illustrates a mean fluorescence intensity (MFI) of ADAR1p150 protein levels in human HSCs (CD45⁺CD34⁺CD38⁻Lin⁻) and HPCs (CD45⁺CD34⁺CD38⁺Lin⁻) from the spleens of sAML50261 engrafted mice treated with Rebecsinib or vehicle;

FIG. 30E illustrates a whole transcriptome-based RNA editing analyses of previously-described RNA-seq data¹⁴ generated from CD34⁺ cells isolated from the spleens of sAML50261 engrafted mice treated with Rebecsinib or vehicle;

FIG. 30F illustrates isoform-level analysis of MCL1 transcripts from RNA-sequencing data shown in FIG. 30E;

FIG. 30G illustrates overall mouse survival in serially transplanted sAML mice (primary transplanted mice were treated with Rebecsinib or vehicle); and

FIG. 30H illustrates ratios of ADAR1p150-3′UTR truncated (ADAR-208) to ADAR1p110 (ADAR-201) by RNA-seq analyses of CD34⁺ cells isolated from serial transplant recipients of sAML50261 LSC engrafted mice treated with vehicle or Rebecsinib.

### In Vivo Mouse Studies

All mouse studies were completed in accordance with the University Laboratory Animal Resources and Institutional Animal Care and Use Committee of the University of California (IACUC) regulations. Rag2^{-/-}γca^{-/-} mice and NSG-SGM3 mice (Jackson Laboratories, Bar Harbor, ME) mice were bred and maintained in the Sanford Consortium vivarium according to IACUC-approved protocols. Rag2^{-/-}γc^{-/-} mice exhibit T cell, B cell, and NK cell immunodeficiencies that make them effective transplant hosts for human immune cells. NSG-SGM3 mice produce 2-4 ng/mL serum levels of human SCF, GM-CSF, and IL-3, which supports the engraftment of myeloid and lymphoid cells. Animals were housed in groups. Mice were randomly assigned to experimental groups based on engraftment levels (equivalent average peripheral blood engraftment levels were present in vehicle and treatment groups prior to initiation of treatment).

### Animals Used in Pre-Clinical Toxicokinetic (TK) Studies

Single-dose TK studies were performed in Sprague Dawley rats (Charles River Labs, South San Francisco, CA), New Zealand white rabbits (BASi/Inotiv, West Lafayette, IN), and cynomolgus monkeys (BASi/Inotiv).

### Human Subjects

Primary patient samples were obtained from consenting patients at the University of California in accordance with a UC San Diego human research protection program Institutional Review Board (IRB)-approved protocol (#131550). The IRB reviewed this protocol and found that it meets the requirements as stated in 45 CFR 46.404 and 21 CFR 50.51. Human cord blood and normal aged-match samples were purchased as purified CD34⁺ cells (AllCells).

### Stromal Co-Culture Assays

Primary patient samples and normal bone marrow controls used for functional assays are described in Table 1. Human HS5 and HS27a⁴⁵ or mouse bone marrow cells lines SL (hSCF and hIL3) and M2 (hIL3 and hG-CSF) were irradiated and then mixed at a ratio of 1:1 and incubated overnight for attachment. To establish co-cultures, 10,000-15,000 CD34⁺ cells were added to SLM2 stroma in 1 ml of MYELOCULT H5100™ (STEMCELL Technologies, Vancouver, Canada). Rebecsinib or DMSO control was added at the initiation of co-culture at indicated concentrations. After one week, cells that were both attached to stroma and floating were collected, resuspended in fresh media and plated in methylcellulose (MC) H4330 (STEMCELL Technologies) in triplicate. After 2 weeks primary colonies (more than 40 cells) were counted and individual multilineage colonies were plucked, cells resuspended, and re-plated again in fresh MC. Secondary colonies were counted after another 14 days. Basal colony formation of untreated cells was considered to be 100% and results are presented as % of change.

### In Vitro Culture of Cell Lines

The following cell lines were used for in vitro lentiviral reporter assays and biomarker development: human KG-1a cells (AML cells derived from a 59 y/o male, cultured in ISCOVE'S MODIFIED DULBECCO'S MEDIUM™, Catalog No. 30-2005, 20% fetal bovine serum), human K562 cells (blast crisis chronic myeloid leukemia cells isolated from a 53 y/o female, cultured in ISCOVE'S MODIFIED DULBECCO'S MEDIUM™, Catalog No. 30-2005, 10% fetal bovine serum), human TF-1a cells (erythroleukemia cells isolated from a 35 y/o male, cultured in RPMI-1640™ medium, ATCC 30-2001, 10% fetal bovine serum), human MOLM-13 (sAML cells isolated from a 20 y/o male, cultured in RPMI-1640 Medium, ATCC 30-2001, 10% fetal bovine serum), human HL-60 cells (acute promyelocytic leukemia cells isolated from a 36 y/o female, cultured in ISCOVE'S MODIFIED DULBECCO'S MEDIUM™, Catalog No. 30-2005, 20% fetal bovine serum), 293T cells (human kidney epithelial cells from human fetus, cultured in DULBECCO'S MODIFIED EAGLE'S MEDIUM™ (DMEM) (ATCC 30-2002, 10% fetal bovine serum), and rat RBL-1 cells (leukemia cells isolated from the Wistar strain, cultured in EAGLE'S MINIMUM ESSENTIAL MEDIUM™, Catalog No. 30-2003, 10% fetal bovine serum). All cell lines, with the exception of MOLM-13 cells, were obtained from the American Type Culture Collections (ATCC, Manassas, VA) and cultured at 37 °C 5% CO₂, and monitored routinely for cell line identity by flow cytometry and for culture integrity by mycoplasma screening.

### Primary Samples, Whole Transcriptome RNA-Sequencing, RNA Editing, and Splice Isoform Analyses

Primary peripheral blood or bone marrow samples from patients with MPNs (n=6 PV, n=2 ET, n=24 MF, n=5 CML) or AML (n=12), along with non-MPN bone marrow controls (n=24) were FACS-purified to isolate live HSC (CD34⁺CD38⁻Lin⁻) and HPC (CD34⁺CD38⁺Lin⁻) populations, as previously described.^{5,14,16} Cells were lysed in RNA extraction buffer and total RNA was extracted using RNAEASY™ micro extraction kits (QIAGEN, Germantown, MD). RNA samples were evaluated for quality. Samples with RNA integrity (RIN) values >7 were processed for whole transcriptome RNA-sequencing (The Scripps Research Institute Next Generation Sequencing Core) on Illumina HISEQ™ platforms.

RNA-Seq was performed on Illumina's NEXTSEQ 500™ sequencer with 150 bp paired-end reads. Transcript quantification was performed as previously described.⁵ Briefly, sequencing data were de-multiplexed and output as fastq files using Illumina's BCL2FASTQ™ (v2.17). Quality control of the raw fastq files was performed using the software tool FASTQC™.⁴⁶ Sequencing reads were aligned to the human genome (hg38) using the STAR v2.5.1a™ aligner.⁴⁷ Read and transcript quantification was performed with RSEM⁴⁸ v1.3.0 and GENCODE annotation (genocode.v19.annotation.gtf). The R BIOCONDUCTOR™ packages EDGER™⁴⁹ and limma⁵⁰ were used to implement the limma-voom⁵¹ method for normalization of transcript levels and calculation of log transformed counts per million (log CPM). For sashimi plots, the .bam alignment files generated with the STAR aligner were input in the INTEGRATED GENOMICS VIEWER™ (IGV v2.12, https://software.broadinstitute.org/software/igv/). The viewer was navigated to the specific ADAR1 region of interest and a sashimi plot was generated with base level coverage represented by individual bars and junction level coverage represented by arcs.

For RNA editing analyses of whole transcriptome sequencing data, previous datasets were utilized from CD34⁺ cells isolated from the spleens of sAML50261-engrafted mice treated once weekly with Rebecsinib in a dose-response assay (5 or 10 mg/kg),¹⁴ along with CD34⁺ cells harvested from mice that were serially transplanted with CD34+ cells from sAML50261-engrafted mice that had been treated twice weekly with Rebecsinib at 10 mg/kg (no further treatment was delivered to serial transplant recipients). Due to limiting number of cells remaining after treatment, cells were pooled from n=4 to 5 mice per condition for sequencing. RNA editing analysis was performed as previously described.⁵

### Lentiviral RNA Splicing and ADAR1 Editing Reporters

To enable real-time, live cell RNA splicing and editing quantification detection, lentiviral dual fluorescence RNA splicing and ADAR1-dependent RNA editing vectors were designed and tested for activity in human leukemia cell lines. To assess ADAR1-dependent editing, a lentiviral reporter was constructed to measure activity by fluorescence and or luminescence. The construct contains a synthetic ADAR1-specific nucleic acid sequence²⁸ (“Herbert sequence”) that was cloned into a pCDH™ (System Biosciences) backbone containing an EF1α promoter and T2A sequence for co-expression of nanoluciferase and COP-GFP. The Herbert sequence precedes a stop codon, UGA, which lies upstream of NANOLUC T2A™ copGFP. Following A-to-I editing, this stop codon is removed, resulting in downstream nanoluciferase and COP-GFP expression. The ADAR1-dependent RNA editing reporter vector was validated in 293T cells via co-transfection with various ADAR-overexpression vectors including ADAR1-FLAG wildtype, ADAR1-FLAG E912A (editing-deficient), ADAR2-FLAG and corresponding backbone. ADAR-overexpression plasmids were transfected at 3 concentrations (0.1 μg, 0.3 μg, 1.0 μg) to demonstrate the sensitivity of ADAR1-dependent editing. Cells were collected for western blot to confirm ADAR overexpression using anti-FLAG M2™ antibody (Sigma F3165). Reporter activity was measured by luminescence using NANO-GLO LUCIFERASE ASSAY SYSTEM™ (Promega).

For generation of stably transduced splicing reporter cell lines, a previously described splicing reporter construct³¹ was cloned into a lentiviral vector backbone.⁵² KG-1a cells were transduced with the dual-fluorescent lentiviral splicing reporter vector, treated with various concentrations of Rebecsinib (3-fold serial dilutions from 3 μM to 4 nM) for 24 hr, washed followed by DAPI staining, and analyzed by flow cytometry for cell viability and RFP/GFP fluorescence intensity. Results were from duplicate wells of each condition. Viability of cells treated with DMSO (0.5% final concentration) was set as 100%. Nonlinear regression curve fit analysis was carried out using Prism software (GraphPad) to determine ECso values for viability and mean fluorescence intensity (MFI) of RFP and GFP.

### In Vitro Cell Line Treatments and Analyses

For lentivirus-mediated knockdown of human ADAR1 in ADAR1 activation and RNA editing biomarker studies of the nanoluc-GFP reporter and endogenous transcripts, human leukemia cells were stably transduced with the lentiviral shRNA vectors targeting ADAR1 (shADAR1) or scrambled control (shCtrl) to ablate endogenous ADAR1 expression. The shRNA plasmids targeting ADAR1 (shADAR1) as well as the scrambled control (shCtrl) were purchased from VectorBuilder (Chicago, IL) (shCtrl: CCTAAGGTTAAGTCGCCCTCG (SEQ ID NO:3) and shADAR1: CCGGACCTCCTCACGAGCCCAAGTTCGTTTACCAAGCAAAA) (SEQ ID NO:4). Human wild-type or catalytically inactive (E912A) mutant vectors were also used to selectively express ADAR1p150 in some experiments. Viral titers were assessed by qRT-PCR and transduction efficiency was tested in 293T cells. 100,000 TF-1a cells stably transduced with the same lentiviral shCtrl (TF-1a shCtrl) and shADAR1 (TF-1a shADAR1) vectors were plated (n=4) and treated with interferon-α or 0.1 μM Rebecsinib or DMSO control for 4 hours.

For ADAR1 protein isoform and STAT3 phosphorylation analyses in cell lines, western blots were performed as previously described.^{5,8,16} Blots were probed with antibodies against ADAR1p150 (Abcam ab126745) and pan-ADAR1 (detects ADAR1p150 and p110, Cell Signaling D7E2M), along with total STAT3, phosphorylated STAT3 (Y705 D3A7, Cell Signaling), and GAPDH as a loading control (Table S2).

### Stromal Co-Cultures and ADAR1 Reporter Assays

Human HS5 and HS27a⁴⁵ or mouse bone marrow cells lines SL (hSCF and hIL3) and M2 (hIL3 and hG-CSF) were irradiated and then mixed at a ratio of 1:1 and incubated overnight for attachment. CD34⁺ were selected from high-risk MF and sAML primary samples using magnetic beads (Miltenyi Biotec, Germany). As a control, CD34⁺ cells from aged normal bone marrow (aNBM), young normal BM (yNBM) and cord blood (All Cells Inc, Alameda, CA) mononuclear cells were utilized. To establish co-cultures, 10,000-15,000 CD34⁺ cells were added to SLM2 stroma in 1 ml of MYELOCULT H5100™ (STEMCELL Technologies, Vancouver, Canada). Rebecsinib or DMSO control were added at the initiation of co-culture at indicated concentrations. After one week, cells that were both attached to stroma and floating were collected, resuspended in fresh media and plated in methylcellulose (MC) H4330™ (STEMCELL Technologies) in triplicate. After 2 weeks primary colonies (more than 40 cells) were counted and individual multilineage colonies were plucked, cells resuspended and re-plated again in fresh MC. Secondary colonies were counted after another 14 days. Basal colony formation of untreated cells was considered to be 100% and results are presented as % of change.

For nanoluciferase RNA editing activity assays, CD34⁺ selected cells from primary high-risk MF samples were lentivirally transduced with ADAR1 nanoluc-GFP reporter for 48 hr, followed by treatment with DMSO or Rebecsinib in stromal co-culture for 72 hr. Luminescence reporter activity was measured in 10,000 cells by NANO-GLO LUCIFERASE ASSAY™ and values were normalized to cell viability. Cell viability was measured in 10,000 cells by CellTiter-Glo Luciferase Assay (Promega). A pilot in vivo reporter assay was also performed using TF-1a cells that were stably transduced with the ADAR1 nanoluc-GFP reporter. For this purpose, cells were co-transduced with lentiviral vectors expressing ADAR1-targeted shRNA (to knock down endogenous wild-type ADAR1) and wild-type or catalytically inactive mutant (E912A) exogenous human ADAR1p150. Cells were transplanted into neonatal Rag2^{-/-}γc^{-/-} mice (70,000 cells per mouse), and animals were imaged 4 weeks after transplant on the IVIS 200, as previously described.^{7,9} Human cell engraftment was also confirmed in the peripheral blood in the same week as imaging.

For normal bone marrow control survival and differentiation assays, CD34⁺ cells from aged bone marrow samples were plated into STEMPRO™ media (ThermoFisher, Carlsbad, CA) and treated with Rebecsinib for 72 hr. Samples were analyzed by flow cytometry using antibodies for total hematopoietic cells (CD45 APC; Life Technologies cat #MHCD4505, 1:50), T cells (CD3 FITC; BIOLEGEND™ cat #300306, 1:20), monocytes (CD14 PerCP-Cy5.5; BD Pharmingen cat #550787, 3:100) and B cells (CD19 PE; BIOLEGEND™ cat #302208, 1:50).

### Splice Isoform Specific Quantitative RT-PCR

For quantitative analysis of splice isoform expression, RNA editing rates, and whole gene expression by qRT-PCR, cells or tissue fragments were harvested in RNA lysis buffer and total RNA was extracted using RNeasy mini or micro extraction kits (QIAGEN, Germantown, MD) following the manufacturer's protocol including a DNase incubation step to digest any trace genomic DNA present. Levels of ADAR1 variants and LSC-specific transcripts were quantified by qRT-PCR as previously described.¹⁴ RNA-editing site-specific qRT-PCR (RESSqPCR) was performed for variants in AZIN1 transcripts.⁵³ Additional species-specific primers were also designed to quantify intron retention rates in cells treated with Rebecsinib as a biomarker of response to RNA splicing modulation. Specifically, for the two-step SYBR-green based assays, as previously described,^{7,14,53} 100-1000 ng of RNA were subjected to cDNA synthesis using the SUPERSCRIPT III™ (ThermoFisher Scientific) kit followed by qRT-PCR using SYBR GreenER (ThermoFisher Scientific) master mix according to the manufacturer's recommended procedures. qRT-PCR was performed using SYBR GREENER SUPER MIX™ (Life Technologies) on BioRad iQ5™, C1000 TOUCH™, or BioRad CFX384™ instruments. Human splice isoform-specific, RNA editing site-specific qPCR (RESSqPCR)⁵³. Primer sets that have not been previously published were designed and tested for efficiency followed by analyses in samples exposed to Rebecsinib, with species-specific HPRT primers used as controls. Samples with Ct<35 for the species-specific reference gene, HPRT, were included in analyses. Relative mRNA expression values were calculated using the 2^{-ΔCT} method, with normalization to untreated or vehicle-treated controls.⁵⁴

For multi-species biomarker development and transcript variant-specific qRT-PCR experiments, human AML cell lines KG-1a, MOLM-13, and HL-60, and rat leukemia cells RBL-1 were grown to confluence and treated with 1 μM Rebecsinib for 4 hr. Cells were collected and lysed in Qiagen RNA lysis buffer, and analyzed using the species-specific primers and qRT-PCR procedures described above.

### Intracellular ADAR1p150 and Phospho-STAT3 Flow Cytometric Analyses

Flow cytometry for stem and progenitor cell surface antibody staining was performed as previously described.¹⁴ For phospho-flow, a subset of samples was fixed and permeabilized, then blocked and stained with APC-conjugated ADAR1p150 antibody (cat ab269444, 1:25) and pSTAT3 APC (cat 17-9033-42, 1:10) (Table S2). Fractions were analyzed with the BD LSR FORTESSA™ (Sanford Consortium for Regenerative Medicine Stem Cell Core) and FLOWJO™ software (TreeStar, Ashland, OR).

### Humanized LSC Mouse Model Assays and Human HSPC Therapeutic Index Studies

In humanized mouse models, sAML cells (CD34⁺) from two unique primate patient samples that were splicing factor mutated or unmutated were utilized, including sAML50261 and sAML2008-5.¹⁴ For in vivo efficacy and therapeutic index studies, sAML CD34⁺ cells, or normal human cord blood-derived or aged bone marrow-derived HSPCs (CD34⁺), were transplanted (50,000-200,000 cells per mouse) intrahepatically into neonatal Rag2^{-/-}γc^{-/-} mice, or intravenously into adult NSG-SGM3 mice (Jackson Laboratories, Bar Harbor, ME). A subset of experiments was performed with normal aged bone marrow cells that were transduced with the lentiviral ADAR1 nanoluc-GFP reporter vector prior to transplant. For transplantation into adult mice, animals were irradiated with ¹³⁷Cs at a dose of 150 cGy 24 hr before IV transplantation. After engraftment levels reached >1% human CD45⁺ hematopoietic cells in the peripheral blood, animals were distributed among treatment groups for treatment with Rebecsinib or vehicle control essentially as previously described, with an increased dosing regimen of twice weekly (versus once weekly in prior studies)¹⁴ and an optimized in vivo formulation (2% w/w EtOH, 5% w/v KOLLIPHOR HS™ 15 in 0.9% saline). For animals that received transplants of cells transduced with the lentiviral ADAR1 nanoluc-GFP reporter, mice were imaged 15 weeks after transplant on the IVIS 200, as previously described.⁷⁹ Following treatment with Rebecsinib (five intravenous doses total over a two-week period), hematopoietic tissues (peripheral blood, bone marrow, spleens) were collected and analyzed by flow cytometry as previously described.¹⁴ For splice isoform-specific qRT-PCR or RNA-seq analyses on cells from in vivo studies, human CD34⁺ LSC from bone marrows and spleens of engrafted mice were isolated by magnetic bead separation (Miltenyi Biotec).

Additional aliquots of CD34+ cells from bone marrow and/or spleens of treated and control animals were used for serial transplantation assays after Rebecsinib treatment, including engraftment studies and overall survival assays. In a separate cohort of animals, mice were treated with Fedratinib for two weeks (twice daily oral delivery at 60 mg/kg) as a positive control for modulation of ADAR1 expression and activity.⁸

### Pre-Clinical Toxicokinetic (TK), Pharmacokinetic and Pharmacodynamic (PD) Studies

Single-dose TK studies were performed in Sprague Dawley rats (Charles River Labs, South San Francisco, CA), New Zealand white rabbits (BASi/Inotiv, West Lafayette, IN), and cynomolgus monkeys (BASi/Inotiv). Rat and rabbit studies included toxicology analyses, quantification of plasma levels of Rebecsinib, and necropsies to evaluate organ integrity after treatment. For NHP studies, health and ophthalmological evaluations were performed before and after dosing with Rebecsinib, and blood samples were collected for quantification of plasma levels of Rebecsinib and biomarker studies in PBMCs from treated and control animals. All monkeys were returned to the animal colony after the end of the observation period. Toxicokinetic values including mean plasma concentrations and t_{1/2} values were calculated according to CRO-approved protocols (Charles River and BASi).

### Statistical Analyses

For ADAR1 and splicing reporter assays, qRT-PCR analyses, and flow cytometry, data were analyzed using Microsoft EXCEL™ and plotted for graph preparation and statistical analyses in PRISM GRAPHPAD™ (San Diego, CA). Differences were assessed by unpaired or paired Student's t-tests, as indicated in the figure legends, and considered statistically significant for p values of <0.05. For stromal co-culture assays and multiple group comparisons, data (means) for summarized sAML, MF or healthy control samples were calculated and graphed. Error bars indicate the SD or SEM, as indicated in individual figure legends. Student's t-test and one-way ANOVA statistical analyses were performed using PRISM GRAPHPAD™ and comparisons described in each figure legend.

### Example 15: Treatment of Spinal Injury

Adult patient presents with trauma to spinal cord, with symptoms of paralysis and numbness peripherally. A therapeutic combination of Rebecsinib and fedratinib (optionally fedratinib hydrochloride capsules, optionally INREBIC™) is administered, each in separate oral formulations, at 400 mg per day, or between 100 mg per day and 500 mg per day, for between one week and two months, or longer if needed, depending on improvements in symptoms. The oral 400 mg per day of fedratinib is maintained provided patient's baseline platelet count is greater than or equal to 50×10⁹/L.

### Example 16: Treatment of Liver Disease and Liver Infection

Adult patient presents with liver disease or infection (viral or parasitic infection, or fasciolosis), or liver cancer or liver infection (optionally hepatitis), or a fatty liver or has fatty liver disease (hepatic steatosis), autoimmune hepatitis, alcoholic hepatitis, or Nonalcoholic Fatty Liver Disease. A therapeutic combination of Rebecsinib and fedratinib (optionally fedratinib hydrochloride capsules, optionally INREBIC™) is administered, each in separate oral formulations, at 400 mg per day, or between 100 mg per day and 500 mg per day, for between one week and two months, or longer if needed, depending on improvements in symptoms. The oral 400 mg per day of fedratinib is maintained provided patient's baseline platelet count is greater than or equal to 50×10⁹/L.

### Example 17: Treatment of Stroke

Adult patient presents with symptoms of recently having a stroke or a thrombo-occlusive cerebrovascular event. A therapeutic combination of Rebecsinib and fedratinib (optionally fedratinib hydrochloride capsules, optionally INREBIC™) is administered, each in separate oral formulations, at 400 mg per day, or between 100 mg per day and 500 mg per day, for between one week and two months, or longer if needed, depending on improvements in symptoms. The oral 400 mg per day of fedratinib is maintained provided patient's baseline platelet count is greater than or equal to 50×10⁹/L.

### REFERENCES EXAMPLE 1

1. Jiang Q, Crews L, Barrett C, et al. ADAR1 promotes malignant progenitor reprogramming in chronic myeloid leukemia. PNAS. 2013; 110(3):1041-1046
2. Zipeto M A, Court A C, Sadarangani A, et al. ADAR1 Activation Drives Leukemia Stem Cell Self-Renewal by Impairing Let-7 Biogenesis. Cell Stem Cell. 2016; 19(2) 177-191.
3. Crews L, Balaian L, Delos Santos, N P et al. RNA Splicing Modulation Selectively Impairs Leukemia Stem Cell Maintenance in Secondary Human AML. Cell Stem Cell. 2016; 19: 599-612
4. Crews L A, Ma W, Ladel L, et al. Reversal of malignant ADAR1 splice isoform switching with Rebecsinib. Cell Stem Cell. 2023; 30(3):250-263.e6

### REFERENCES EXAMPLE 3

Inflammation-driven deaminase deregulation fuels human pre-leukemia stem cell evolution, Jiang Q. et al., 2021, Cell Reports.
ADAR1 Transcriptome editing promotes breast cancer progression through the regulation of cell cycle and DNA damage response, Sagredo E. A. et al., 2020, BBA-Molecular Cell Research.
Web-Based Survival Analysis Tool Tailored for Medical Research (KMplot): Development and implementation, Lanczky A. and Gyorffy B., 2021 J Med Internet Res.
Reversal of malignant ADAR1 splice isoform switching with Rebecsinib, Crews A. L., et al., 2023. Cell Stem Cell
Detection and targeting of splicing deregulation in pediatric acute myeloid leukemia stem cells, Van der Werf I., et al., 2023. Cell Reports Medicine.
A CD47-associated super-enhancer links pro-inflammatory signalling to CD47 upregulation in breast cancer, Betancur P. A. et al., 2017, Nature Communications.

### REFERENCES EXAMPLE 14

1. Schoggins, J. W., et al. (2011). A diverse range of gene products are effectors of the type I interferon antiviral response. Nature 472, 481-485. 10.1038/nature09907.
2. Sanchez-Rivera, et al. (2022) Base editing sensor libraries for high-throughput engineering and functional analysis of cancer-associated single nucleotide variants. Nat Biotechnol 40, 862-873. 10.1038/s41587-021-01172-3.
3. Porto, E. M., et al. (2020). Base editing. Nat Rev Drug Discov 19, 839-859. 10.1038/s41573-020-0084-6.
4. Fry, L. E., et al. (2020). RNA editing as a therapeutic approach for retinal gene therapy requiring long coding sequences. Int J Mol Sci 21. 10.3390/ijms21030777.
5. Jiang, Q., et al. (2021). Inflammation-driven deaminase deregulation fuels human pre-leukemia stem cell evolution. Cell Rep 34, 108670. 10.1016/j.celrep.2020.108670.
6. Jiang, Q., et al. (2019). A-to-I RNA editing in leukemia stem cells—set ADAR1 on the radar. Oncotarget 10, 6047-6048. 10.18632/oncotarget.27261.
7. Lazzari, E., et al. (2017). Alu-dependent RNA editing of GLIl promotes malignant regeneration in multiple myeloma. Nat Commun 8, 1922. 10.1038/s41467-017-01890-w.
8. Zipeto, Maria A., et al. (2016). ADAR1 Activation Drives Leukemia Stem Cell Self-Renewal by Impairing Let-7 Biogenesis. Cell Stem Cell 19, 177-191. 10.1016/j.stem.2016.05.004.
9. Jiang, Q., et al. (2013). ADAR1 promotes malignant progenitor reprogramming in chronic myeloid leukemia. Proc Natl Acad Sci USA 110, 1041-1046. 10.1073/pnas.1213021110.
10. Xu, L. D., and Ohman, M. (2018). ADAR1 Editing and its Role in Cancer. Genes (Basel) 10. 10.3390/genes10010012.
11. Abrahamsson, A. E., et al. (2009). Glycogen synthase kinase 3beta missplicing contributes to leukemia stem cell generation. Proc Natl Acad Sci USA 106, 3925-3929. 10.1073/pnas.0900189106.
12. Goff, D. J., et al. (2013). A Pan-BCL2 inhibitor renders bone-marrow-resident human leukemia stem cells sensitive to tyrosine kinase inhibition. Cell Stem Cell 12, 316-328. 10.1016/j.stem.2012.12.011.
13. Holm, F., et al. (2015). Reversion to an embryonic alternative splicing program enhances leukemia stem cell self-renewal. Proc Natl Acad Sci USA 112, 15444-15449. 10.1073/pnas.1506943112.
14. Crews, L. A., et al. (2016). RNA Splicing Modulation Selectively Impairs Leukemia Stem Cell Maintenance in Secondary Human AML. Cell Stem Cell 19, 599-612. 10.1016/j.stem.2016.08.003.
15. Jiang, Q., et al. (2017). RNA editing-dependent epitranscriptome diversity in cancer stem cells. Nat Rev Cancer 17, 381-392. 10.1038/nrc.2017.23.
16. Jiang, Q., et al. (2019). Hyper-Editing of Cell-Cycle Regulatory and Tumor Suppressor RNA Promotes Malignant Progenitor Propagation. Cancer Cell 35, 81-94 e87. 10.1016/j.ccell.2018.11.017.
17. Hartner, J. C., et al. (2009). ADAR1 is essential for the maintenance of hematopoiesis and suppression of interferon signaling. Nat Immunol 10, 109-115. ni.1680 [pii]10.1038/ni.1680.
18. Lu, S. X., et al. (2021). Pharmacologic modulation of RNA splicing enhances anti-tumor immunity. Cell 184, 4032-4047 e4031. 10.1016/j.cell.2021.05.038.
19. Ishizuka, J. J., et al. (2019). Loss of ADAR1 in tumours overcomes resistance to immune checkpoint blockade. Nature 565, 43-48. 10.1038/s41586-018-0768-9.
20. Zhang, T., et al. (2022). ADAR1 masks the cancer immunotherapeutic promise of ZBP1-driven necroptosis. Nature 606, 594-602. 10.1038/s41586-022-04753-7.
21. Chan, T. H., et al. (2014). A disrupted RNA editing balance mediated by ADARs (Adenosine DeAminases that act on RNA) in human hepatocellular carcinoma. Gut 63, 832-843. 10.1136/gutjnl-2012-304037.
22. Qin, Y. R., et al. (2014). Adenosine-to-inosine RNA editing mediated by ADARs in esophageal squamous cell carcinoma. Cancer Res 74, 840-851. 10.1158/0008-5472.CAN-13-2545.
23. Han, L., et al. (2015). The Genomic Landscape and Clinical Relevance of A-to-I RNA Editing in Human Cancers. Cancer Cell 28, 515-528. 10.1016/j.ccell.2015.08.013.
24. Lykke-Andersen, S., et al. (2007). Alternative splicing of the ADAR1 transcript in a region that functions either as a 5′-UTR or an ORF. RNA 13, 1732-1744. 10.1261/rna.567807.
25. Licht, K., et al. (2016). Adenosine to Inosine editing frequency controlled by splicing efficiency. Nucleic Acids Res. 10.1093/nar/gkw325.
26. Dharshini, S. A. P., et al. (2020). Identifying suitable tools for variant detection and differential gene expression using RNA-seq data. Genomics 112, 2166-2172. 10.1016/j.ygeno.2019.12.011.
27. Cretu, C., et al. (2018). Structural Basis of Splicing Modulation by Antitumor Macrolide Compounds. Molecular Cell 70, 265-273.e268. 10.1016/j.molcel.2018.03.011.
28. Herbert, A., W et al. (2002). Induction of protein translation by ADAR1 within living cell nuclei is not dependent on RNA editing. Mol Cell 10, 1235-1246. S1097276502007372 [pii].
29. Fritzell, K., et al. (2019). Sensitive ADAR editing reporter in cancer cells enables high-throughput screening of small molecule libraries. Nucleic Acids Res 47, e22. 10.1093/nar/gky1228.
30. Qian, Y., et al. (2022). Programmable RNA sensing for cell monitoring and manipulation. Nature 610, 713-721. 10.1038/s41586-022-05280-1.
31. Stoilov, P., et al. (2008). A high-throughput screening strategy identifies cardiotonic steroids as alternative splicing modulators. Proc Natl Acad Sci USA 105, 11218-11223. 0801661105 [pii]10.1073/pnas.0801661105.
32. Teoh, P. J., et al. (2020). IL6R-STAT3-ADAR1 (P150) interplay promotes oncogenicity in multiple myeloma with 1q21 amplification. Haematologica 105, 1391-1404. 10.3324/haematol.2019.221176.
33. Kumar, D., et al. (2016). Selectivity in Small Molecule Splicing Modulation. ACS Chem Biol 11, 2716-2723. 10.1021/acschembio.6b00399.
34. Chan, W., et al. (2020). Scalable Synthesis of 17S-FD-895 Expands the Structural Understanding of Splice Modulatory Activity. Cell Reports Physical Science 100277.
35. Siegel, R., et al. (2014). Cancer statistics, 2014. CA Cancer J Clin 64, 9-29. 10.3322/caac.21208.
36. Siegel, R. L., et al. (2017). Cancer Statistics, 2017. CA Cancer J Clin 67, 7-30. 10.3322/caac.21387.
37. Mesa, R. A., et al. (2017). NCCN Guidelines Insights: Myeloproliferative Neoplasms, Version 2.2018. J Natl Compr Canc Netw 15, 1193-1207. 10.6004/jnccn.2017.0157.
38. Hsu, T. Y., et al. (2015). The spliceosome is a therapeutic vulnerability in MYC-driven cancer. Nature 525, 384-388. 10.1038/nature14985.
39. Lee, S. C., et al. (2016). Modulation of splicing catalysis for therapeutic targeting of leukemia with mutations in genes encoding spliceosomal proteins. Nat Med. 10.1038/nm.4097.
40. Dvinge, H., et al. (2016). RNA splicing factors as oncoproteins and tumour suppressors. Nat Rev Cancer 16, 413-430. 10.1038/nrc.2016.51.
41. Steensma, D. P., et al. (2021). Phase I First-in-Human Dose Escalation Study of the oral SF3B1 modulator H3B-8800 in myeloid neoplasms. Leukemia 35, 3542-3550. 10.1038/s41375-021-01328-9.
42. Eskens, F. A., et al. (2013). Phase I pharmacokinetic and pharmacodynamic study of the first-in-class spliceosome inhibitor E7107 in patients with advanced solid tumors. Clin Cancer Res 19, 6296-6304. 10.1158/1078-0432.CCR-13-0485.
43. Hong, D. S., et al. (2014). A phase I, open-label, single-arm, dose-escalation study of E7107, a precursor messenger ribonucleic acid (pre-mRNA) splicesome inhibitor administered intravenously on days 1 and 8 every 21 days to patients with solid tumors. Invest New Drugs 32, 436-444. 10.1007/s10637-013-0046-5.
44. León, B., et al. (2017). A Challenging Pie to Splice: Drugging the Spliceosome. Angew Chem Int Ed Engl 56, 12052-12063. 10.1002/anie.201701065.
45. Roecklein, B. A., and Torok-Storb, B. (1995). Functionally distinct human marrow stromal cell lines immortalized by transduction with the human papilloma virus E6/E7 genes. Blood 85, 997-1005.
46. Andrews, S. (2010). FastQC: a quality control tool for high throughput sequence data. Babraham Bioinformatics.
47. Dobin, A., et al. (2013). STAR: ultrafast universal RNA-seq aligner. Bioinformatics 29, 15-21. 10.1093/bioinformatics/bts635.
48. Li, B., and Dewey, C. N. (2011). RSEM: accurate transcript quantification from RNA-Seq data with or without a reference genome. BMC Bioinformatics 12, 323. 10.1186/1471-2105-12-323.
49. Robinson, M. D., et al. (2010). edgeR: a Bioconductor package for differential expression analysis of digital gene expression data. Bioinformatics 26, 139-140. 10.1093/bioinformatics/btp616.
50. Ritchie, M. E., et al. (2015). limma powers differential expression analyses for RNA-sequencing and microarray studies. Nucleic Acids Res 43, e47. 10.1093/nar/gkv007.
51. Law, C. W., et al. (2014). voom: Precision weights unlock linear model analysis tools for RNA-seq read counts. Genome Biol 15, R29. 10.1186/gb-2014-15-2-r29.
52. van der Werf, I., et al. (2022). Detection and Targeting of Splicing Deregulation in Pediatric Acute Myeloid Leukemia Stem Cells. Submitted.
53. Crews, L. A., et al. (2015). An RNA editing fingerprint of cancer stem cell reprogramming. J Transl Med 13, 52. 10.1186/s12967-014-0370-3.
54. Bustin, S. A., et al. (2009). The MIQE guidelines: minimum information for publication of quantitative real-time PCR experiments. Clin Chem 55, 611-622. 10.1373/clinchem.2008.112797.

A number of embodiments of the invention have been described. Nevertheless, it can be understood that various modifications may be made without departing from the spirit and scope of the invention. Accordingly, other embodiments are within the scope of the following claims.

1: A method for:
promoting in vivo expansion of human CD34⁺ cells and sparing CD45⁺ cell survival in vivo in an individual in need thereof,
increasing human CD34⁺ cells and normal hematopoietic stem cells (HSCs) in an in vivo bone marrow microenvironment in an individual in need thereof,
treating, preventing or ameliorating a spinal cord injury, liver cirrhosis and/or peripheral vascular disease, and/or for promoting neuronal regeneration, in an individual in need thereof,
treating, preventing or ameliorating a stroke, or treating, preventing or ameliorating a thrombo-occlusive cerebrovascular disease in an individual in need thereof,
promoting normal hematopoietic stem cell retention in the bone marrow niche in vivo in an individual in need thereof,
treating a JAK2-related disease or a ADAR-related disease in an individual in need thereof, or
treating, preventing or ameliorating a cancer, neoplasm or tumor in an individual in need thereof,
the method comprising administering to an individual in need thereof a drug combination comprising: rebecsinib, or rebecsinib and fedratinib.

2: The method of claim 1, wherein the in vivo expansion of human CD34⁺ cells and sparing CD45⁺ cell survival in vivo is used for treating, preventing or ameliorating a spinal cord injury, liver cirrhosis and/or a peripheral vascular disease, and/or for promoting neuronal regeneration.

3: The method of claim 1, wherein the cancer, neoplasm or tumor is a cancer lacking receptors for estrogen, progesterone and/or HER2 (human epidermal growth factor receptor 2, or lacking CD340 (cluster of differentiation 340)), or is a triple negative breast cancer.

4: The method of claim 1, wherein doses of rebecsinib, or rebecsinib and fedratinib, are administered, or formulated for administration, once a day for between one to two weeks, twice a week for 2 weeks or between about one to two weeks, followed by 2 weeks rest or 2 to 4 weeks rest, with a duration of two, three, four, five or six cycles, optionally with a duration of four 28 day or monthly cycles.

5: The method of claim 1, further comprising administering to an individual in need thereof an ATP-competitive protein tyrosine kinase inhibitor.

6: The method of claim 1, wherein doses of rebecsinib, or rebecsinib and fedratinib, are dosaged at about 60 mg/kg twice daily orally, or between about 20 mg to 100 mg, optionally for one to two, or three, or four, or five or more weeks, or is dosaged at between about 10 to 500 mg/day, or between about 500 to 1 gram a day, or at a dosage of between about 100 to 600 mg per day or per dosage, or at about 100, 200, 300, 400, 500 or 600 mg per day or per dosage.

7: The method of claim 1, further comprising administering to the individual in need thereof one, two, three or more of: a hypomethylating agent (HMA), wherein optionally the HMA comprises azacitidine or decitabine, afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib, vincristine, vinblastine, vinorelbine, and vindesine.

8: The method of claim 1, further comprising administering to the individual in need thereof comprises a telomerase inhibitor, wherein optionally the telomerase inhibitor comprises at least one, two or three of: imetelstat, zidovudine (or azidothymidine (AZT)), stavudine (or ZERIT™), tenofovir or tenofovir disoproxil (or VIREAD™), didanosine (or VIDEX™), abacavir (ZIAGEN™), TMPI, telomestatin, RHPS4, BRACO-19, TMPyP4, tertomotide, ASTVAC-1, GX-301, UCPVax, UV-1, Vx-001, Vx-006, INO-1400, INVAC-1, ASTVAC-2, Telin(ab 4,4-dichloro-1-(2,4-dichlorophenyl)-3-methyl-5-pyrazolone), Vbx-011, Vbx-021, Vbx-026INO-5401, KML-001, TK-005, ribovax, Vbx-016, ZI-HX, ZI-H04, and ZIH-03.

9: The method of claim 1, wherein the formulation, pharmaceutical composition or therapeutic combination of drugs or an active agent or drug contained therein administered to the individual in need thereof is or are formulated or contained in: a liquid formulation (optionally sterile saline or water), a spray, a powder, an aerosol, a mist, or any formulation for inhalation, a pill, a capsule, a tablet, or a geltab, or equivalents; or, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; or, a drug delivery device or package, blister pack, clamshell or tray comprising a plurality of compartments spatially arranged on the drug delivery device or package, blister pack, clamshell or tray to follow a dosage administration regimen.

10: The method of claim 1, wherein an active agent or drug in the formulation, pharmaceutical composition or therapeutic combination of drugs administered to the individual in need thereof is dosaged at between about 10 to 500 mg/day, or between about 500 to 1 gram a day, or at a dosage of between about 100 to 600 mg per day or per dosage, or at about 100, 200, 300, 400, 500 or 600 mg per day or per dosage, and optionally a unit dosage is administered to an individual in need thereof once a day (QD), or twice a day (BID), or three times a day (TID), or more.

11: The method of claim 1, wherein an active agent or drug in the formulation, pharmaceutical composition or therapeutic combination of drugs administered to the individual in need thereof is administered as or formulated with or formulated as an) inhaled or aerosol formulation such as a powder or a mist or aerosol, and/or is formulated with or formulated as an oral, intramuscular (IM), subcutaneous (SC), intrathecal or intravenous (IV) formulation, wherein optionally both the inhaled (or aerosol) and the oral, IV, SC, intrathecal and/or IM formulations are administered simultaneously or sequentially.

12: The method of claim 1, wherein an active agent or drug in the formulation, pharmaceutical composition or therapeutic combination of drugs administered to the individual in need thereof using a drug delivery device, optionally by inhalation, wherein the drug delivery device optionally comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device, or intravenously (IV) or intramuscularly (IM).

13: A drug combination comprising: rebecsinib and fedratinib.

14. (canceled)

15: The method of claim 1, wherein the individual in need thereof is an individual that has or has had an acute or chronic neurodegeneration or disease associated with a neurodegenerative pathology.

16: The method of claim 1, wherein the individual in need thereof is an individual that has or has had Parkinson's disease or Alzheimer's disease, or traumatic brain injury (TBI) or Chronic Traumatic Encephalopathy (CTE), or chemical brain or nerve damage,

17: The method of claim 1, wherein the individual in need thereof is an individual that has or has had a liver disease or infection, or a liver cancer or liver infection, or a fatty liver or has fatty liver disease or hepatic steatosis, autoimmune hepatitis, alcoholic hepatitis, or Nonalcoholic Fatty Liver Disease.

18: The method of claim 1, wherein the liver disease or infection comprises a viral or parasitic infection, or fasciolosis.

19: The method of claim 1, wherein the liver infection is a hepatitis infection.

20: The method of claim 5, wherein the ATP-competitive protein tyrosine kinase inhibitor comprises dasatinib.

21: The method of claim 6, wherein a unit dosage is administered to an individual in need thereof once a day (QD), or twice a day (BID), or three times a day (TID), or more.
